(19) United States
(12) Patent Application Publication
(10) Pub. No.: US 2013/0041025 A1
(43) Pub. Date: Feb. 14, 2013

(54) COMPOSITIONS AND METHODS FOR TREATING HAIR LOSS, HAIR THINNING, AND HAIR COLOR LOSS

(71) Applicants: John G. Walt, Huntington Beach, CA (US); David F. Woodward, Lake Forest, CA (US); Amanda M. Vandenburgh, Huntington Beach, CA (US)

(72) Inventors: John G. Walt, Huntington Beach, CA (US); David F. Woodward, Lake Forest, CA (US); Amanda M. Vandenburgh, Huntington Beach, CA (US)

(73) Assignee: ALLERGAN, INC., Irvine, CA (US)

(21) Appl. No.: 13/069,665

(22) Filed: Mar. 23, 2011

Related U.S. Application Data
(63) Continuation-in-part of application No. 12/425,933, filed on Apr. 17, 2009, now Pat. No. 8,298,518.
Application No. 12/425,933 is a continuation of application No. 11/943,714, filed on Nov. 21, 2007, now Pat. No. 8,038,988.
Application No. 11/943,714 is a continuation of application No. 11/805,122, filed on May 22, 2007, now Pat. No. 8,101,161.
Application No. 11/805,122 is a continuation of application No. 10/345,788, filed on Jan. 15, 2003, now Pat. No. 7,351,404.
(60) Provisional application No. 61/316,967, filed on Mar. 24, 2010.
(60) Provisional application No. 60/354,425, filed on Feb. 4, 2002.

(51) Int. Cl. A61K 31/165; A61K 31/16; A61K 31/357; A61P 17/14

(52) U.S. Cl. 514/469; 514/622; 514/613

(57) ABSTRACT

Described herein are compositions including prostaglandin analogs including prostamides and methods for using these compositions for increasing eyelash and eyebrow growth and hair growth on the scalp.

### CROSS REFERENCE TO RELATED APPLICATIONS

This application is a Continuation-in-Part of U.S. patent application Ser. No. 12/425,933 filed on Apr. 17, 2009, which is a continuation of U.S. patent application Ser. No. 11/943,714, filed Nov. 21, 2007, which is a continuation of U.S. patent application Ser. No. 11/805,122, filed May 22, 2007, which is a continuation of U.S. Pat. No. 7,351,404, filed on Jan. 15, 2003, which claims the benefit of U.S. Provisional Application No. 60/354,425, filed on Feb. 4, 2002. This application also claims the benefit of U.S. Provisional Application No. 61/316,967, filed on Mar. 24, 2010. Each application is hereby incorporated by reference herein, the entire disclosures of which are incorporated herein by reference.

### BACKGROUND

Dermatologists recognize many different types of hair loss. Although the most common form is androgenetic alopecia, i.e., male pattern baldness, hair loss does not only occur in men. In fact, hair loss is a problem for both men and women of all ages. Hair loss can result from a variety of causes, such as, but not limited to hormonal changes during pregnancy and childbirth, disease (hyper- and hypo-thyroidism, lupus, trichotillomania), medication, chemotherapy, dietary deficiencies, and stress. In certain instances, the cause of hair loss is unknown.

At present there is no cure for hair loss. An agent that safely and effectively reduces hair loss due to any cause or increases hair growth would be highly desirable.

### SUMMARY

Aspects of the present specification disclose prostaglandins, prostamides, PG glycol esters, and an analogs thereof. Prostaglandins include, without limitation, a PGD₂, a PGE₂, a PGF_{2α}, a 11β-PGF_{2α}, a PGG₂, a PGH₂, a PGI₂, a prostacyclin, or a thromboxane A₂. Prostamides include, without limitation, a prostamide D₂ (PGD₂-ethanolamide), prostamide E₂ (PGE₂-ethanolamide), a prostamide F_{2α} (PGF_{2α}-ethanolamide), a 11β-prostamide F_{2α} (11β-PGF_{2α}-ethanolamide), a prostamide G₂ (PGG₂-ethanolamide), a prostamide H₂ (PGH₂-ethanolamide), or a prostamide I₂ (PGI₂-ethanolamide). Prostaglandin-glycerol esters include, without limitation, a PGD₂-glycerol ester, a PGE₂-glycerol ester, a PGF_{2α}-glycerol ester, a 11β-PGF_{2α}-glycerol ester, a PGG₂-glycerol ester, a PGH₂-glycerol ester, a PGI₂-glycerol ester, a prostacyclin-glycerol ester, or a thromboxane A₂-glycerol ester. A prostaglandin analog, prostamide analog, or PG-glycerol ester analog comprises a compound having the structure:

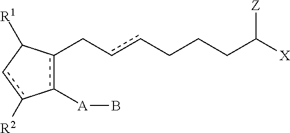

wherein the dashed bonds represent a single or double bond which can be in the cis or trans configuration;
X is —OR⁴ or —N(R⁴)₂ wherein R⁴ is selected from hydrogen, a lower alkyl radical having from one to six carbon atoms, CH(OH)₂,

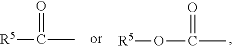

wherein R⁵ is a lower alkyl radical having from one to six carbon atoms;
Z is =0 or represents two hydrogen radicals;
one of R¹ and R² is =0, —OH or a —O(CO)R⁶ group, and the other one is —OH or —O(CO)R⁶, or R¹ is =0 and R² is hydrogen, wherein R⁶ is a saturated or unsaturated acyclic hydrocarbon group having from 1 to about 20 carbon atoms, or —(CH₂)ₘR⁷, wherein m is 0 or an integer of from 1 to 10, and R⁷ is cycloalkyl radical, having from three to seven carbon atoms, or a hydrocarbyl aryl or heteroaryl radical, as defined above;
A is an alkylene or alkenylene radical having from two to six carbon atoms, which radical may be interrupted by one or more oxide radicals and substituted with one or more hydroxy, oxo, alkyloxy or alkylcarboxy groups wherein the alkyl radical comprises from one to six carbon atoms; and
B is a cycloalkyl radical having from three to seven carbon atoms, an aryl radical, selected from hydrocarbyl aryl and heteroaryl radicals having from four to ten carbon atoms wherein the heteroatom is selected from nitrogen, oxygen and sulfur atoms.

Other aspects of the present specification disclose compositions comprising a prostaglandin, a prostamide, a PG glycol ester, or an analog thereof. A composition comprising a prostaglandin, a prostamide, a PG glycol ester, or an analog thereof can be a pharmaceutical composition. Such a pharmaceutical composition can comprise, in addition to a prostaglandin, a prostamide, a PG glycol ester, or an analog thereof, a pharmaceutical carrier, a pharmaceutical component, or both.

Yet other aspects of the present specification disclose a method for treating alopecia in an individual in need thereof comprising the step of administering a therapeutically effective amount of a composition comprising a prostaglandin, prodrug thereof, salt thereof, or mixtures thereof to the individual, wherein the administration results in a reduction in alopecia.

Yet other aspects of the present specification disclose a method for treating hair thinning in an individual in need thereof comprising the step of administering a therapeutically effective amount of a composition comprising a prostaglandin, prodrug thereof, salt thereof, or mixtures thereof to the individual, wherein the administration results in a reduction in hair thinning.

Yet other aspects of the present specification disclose a method for treating hair color loss in an individual in need thereof comprising the step of administering a therapeutically effective amount of a composition comprising a prostaglandin, prodrug thereof, salt thereof, or mixtures thereof to the individual, wherein the administration results in a reduction in hair color loss.

Yet other aspects of the present specification disclose a method for treating an attribute associated with hair loss, hair thinning, or hair color loss in an individual in need thereof comprising the step of administering a therapeutically effective amount of a composition comprising a prostaglandin, prodrug thereof, salt thereof, or mixtures thereof to the individual, wherein the administration results in a reduction in the attribute associated with hair loss, hair thinning, or hair color loss. Attributes associated with hair loss, hair thinning, and/or hair color loss include, without limitation, no new hair shaft growth, reduced rate of hair shaft growth, reduced hair shaft diameter (thickness), reduced hair shaft length, reduced hair density, reduced hair pigmentation, reduced melanin production, decreased keratinization of the hair shaft, reduced hair shaft luster, reduced hair health, increased fragility of the hair shaft, reduced time a hair follicle spends in anagen phase, reduced time a hair follicle spends in catagen phase, reduced time a hair follicle spends in telogen phase, premature release of hair shaft from hair follicle in exogen phase, premature initiation of apoptosis in hair follicle, premature conversion of a terminal hair into a vellus hair.

Still other aspects of the present specification disclose a method for treating alopecia in an individual in need thereof comprising the step of administering a therapeutically effective amount of a composition comprising a prostamide, prodrug thereof, salt thereof, or mixtures thereof to the individual, wherein the administration results in a reduction in alopecia.

Still other aspects of the present specification disclose a method for treating hair thinning in an individual in need thereof comprising the step of administering a therapeutically effective amount of a composition comprising prostamide, prodrug thereof, salt thereof, or mixtures thereof to the individual, wherein the administration results in hair thinning.

Still other aspects of the present specification disclose a method for treating hair color loss in an individual in need thereof comprising the step of administering a therapeutically effective amount of a composition comprising a prostamide, prodrug thereof, salt thereof, or mixtures thereof to the individual, wherein the administration results in a reduction in hair color loss.

Still other aspects of the present specification disclose a method for treating an attribute associated with hair loss, hair thinning, or hair color loss in an individual in need thereof comprising the step of administering a therapeutically effective amount of a composition comprising a prostamide, prodrug thereof, salt thereof, or mixtures thereof to the individual, wherein the administration results in a reduction in the attribute associated with hair loss, hair thinning, or hair color loss. Attributes associated with hair loss, hair thinning, and/or hair color loss include, without limitation, no new hair shaft growth, reduced rate of hair shaft growth, reduced hair shaft diameter (thickness), reduced hair shaft length, reduced hair density, reduced hair pigmentation, reduced hair shaft luster, reduced hair health, reduced time a hair follicle spends in anagen phase, reduced time a hair follicle spends in catagen phase, reduced time a hair follicle spends in telogen phase, premature release of hair shaft from hair follicle in exogen phase, premature initiation of apoptosis in hair follicle, premature conversion of a terminal hair into a vellus hair.

Further aspects of the present specification disclose a method for treating alopecia in an individual in need thereof comprising the step of administering a therapeutically effective amount of a composition comprising a prostaglandin-glycerol ester, prodrug thereof, salt thereof, or mixtures thereof to the individual, wherein the administration results in a reduction in alopecia.

Further aspects of the present specification disclose a method for treating hair thinning in an individual in need thereof comprising the step of administering a therapeutically effective amount of a composition comprising a prostaglandin-glycerol ester, prodrug thereof, salt thereof, or mixtures thereof to the individual, wherein the administration results in a reduction in hair thinning.

Further aspects of the present specification disclose a method for treating hair color loss in an individual in need thereof comprising the step of administering a therapeutically effective amount of a composition comprising a prostaglandin-glycerol ester, prodrug thereof, salt thereof, or mixtures thereof to the individual, wherein the administration results in a reduction in hair color loss.

Further aspects of the present specification disclose a method for treating an attribute associated with hair loss, hair thinning, or hair color loss in an individual in need thereof comprising the step of administering a therapeutically effective amount of a composition comprising a PG-glycerol ester, prodrug thereof, salt thereof, or mixtures thereof to the individual, wherein the administration results in a reduction in the attribute associated with hair loss, hair thinning, or hair color loss. Attributes associated with hair loss, hair thinning, and/or hair color loss include, without limitation, no new hair shaft growth, reduced rate of hair shaft growth, reduced hair shaft diameter (thickness), reduced hair shaft length, reduced hair density, reduced hair pigmentation, reduced hair shaft luster, reduced hair health, reduced time a hair follicle spends in anagen phase, reduced time a hair follicle spends in catagen phase, reduced time a hair follicle spends in telogen phase, premature release of hair shaft from hair follicle in exogen phase, premature initiation of apoptosis in hair follicle, premature conversion of a terminal hair into a vellus hair.

### BRIEF DESCRIPTION OF THE DRAWINGS

FIGS. 1A and B are graphs illustrating the percentage of respondents responding to the question “Do your eyes sting or burn upon application?” Respondents who indicated “yes” were asked about the degree (A) and duration (B) of how bothersome. The percentage of all respondents giving each answer is shown.

FIG. 2 is a graph illustrating the subject responses to “Compared to your first visit, how are your eyelashes now?” The percentage of respondents giving each answer at each time point is shown. At week 4, there were 19 respondents, 18 responded at week 8 and 16 responded at week 12.

FIG. 3 is a graph illustrating subject responses to “Overall the treatment was helpful.” The percentage of respondents giving each answer at each time point is shown. At week 4, there were 19 respondents, 18 responded at week 8 and 16 responded at week 12.

FIG. 4 is a graph illustrating subject responses to “I did something positive for my appearance.” The percentage of respondents giving each answer at each time point is shown. At week 4, there were 18 respondents, 18 responded at week 8 and 16 responded at week 12.

FIG. 5 is a graph illustrating the percentage of subjects that exhibited at least a 1-Grade improvement in global eyelash assessment (GEA).

FIG. 6 is a graph illustrating the mean change (y axis left-pixels, right mm) from baseline over 20 weeks in the intent-to-treat population.

FIG. 7 is a graph illustrating the mean change from baseline of progressive eyelash thickness (% AOI) in the intent-to-treat population.

FIG. 8 is a graph illustrating the mean change in eyelash darkness (spline) from baseline in the intent-to-treat population.

FIG. 9 is a diagram illustrating the biosynthetic pathways of prostaglandins, prostamides, and prostaglandin-glycerol esters.

FIG. 10 is a diagram illustrating anandamide conversion pathways.

### DETAILED DESCRIPTION

Described herein are methods for treating a condition associated with hair loss, hair thinning or hair color loss. The methods generally comprise administering an effective amount of a prostamide, PG glycol ester, prostaglandin, or an analog thereof to an epidermal region of an individual thereby increasing new hair growth, increasing the rate of hair growth, increasing hair thickness, increasing hair length, increasing hair density, increasing number of hair shafts produce per follicle, increasing hair pigmentation, increasing hair luster, converting intermediate or vellus hair to terminal hair, increasing hair health, increasing the time a hair follicle remains in anagen phase, increasing the time a hair follicle remains in catagen phase, increasing the time a hair follicle remains in telogen phase, or preventing the release of a hair shaft in the exogen phase.

The hair growth enhancing compositions disclosed herein was discovered during unilateral treatment of patients with glaucoma. It was noted that the treated eye had noticeably longer, thicker, fuller eyelashes compared to the lashes on the untreated eye. These findings, which are disclosed in U.S. Pat. No. 7,351,404 and are incorporated herein by reference, were unexpected and surprising and led to the development of methods for enhancing hair growth as described herein.

Aspects of the present specification disclose, in part, protaglandins, prostamides, prostaglandin-glycerol esters, and analogs thereof. It is within the scope of the present specification that pharmaceutically acceptable salts of, derivatives of, prodrugs of, tautomers of, and combinations of the prostaglandins, prostamides, and prostaglandin-glycerol esters described herein are useful.

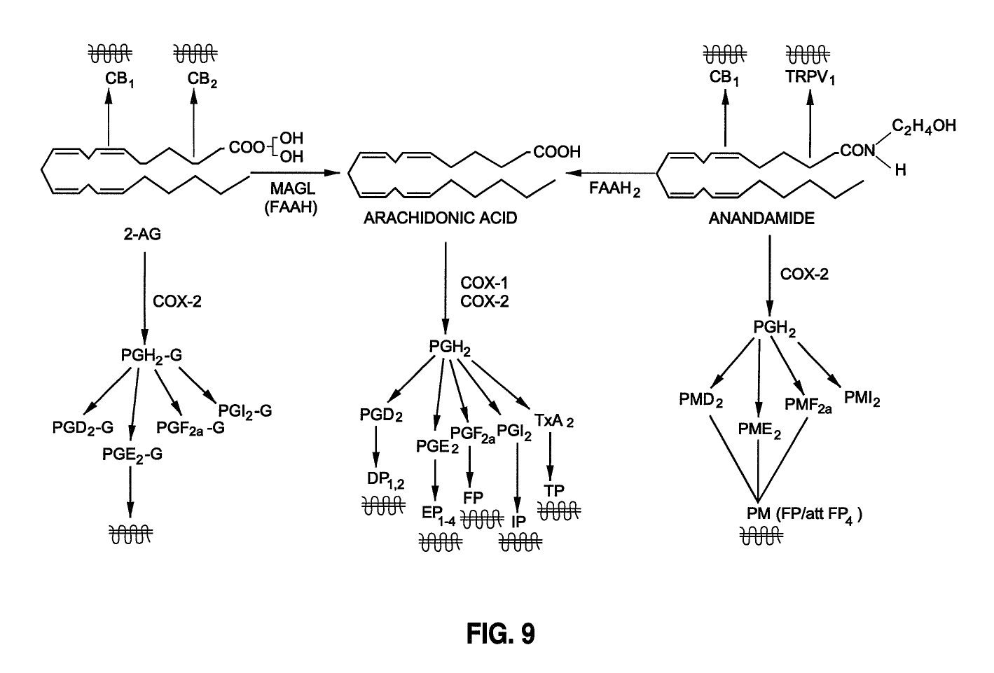

Protaglandins (PGs), prostamides, and prostaglandin-glycerol esters (PG-glycerol esters or PG-glyceryl esters) are derivatives of endocannabinoids/endovanniloids (FIG. 9). Prostaglandins are a family of a group of lipid compounds that are derived enzymatically in the body from essential fatty acids. Every prostaglandin contains 20 carbon atoms, including a 5-carbon ring. Prostaglandins have a wide variety of effects, including, but not limited to, muscular constriction, mediating inflammation, calcium movement, hormone regulation and cell growth control.

Prostamides are prostaglandin-ethanolamides. The pharmacology of prostamide appears unique and bears little resemblance to the pharmacology of their corresponding prostaglandins. See e.g., R. M. Burk and D. F. Woodward, A historical perspective and recent advances in prostamide research and therapeutics. Curr. Opin. Drug Discov. Devel. 10(4): 413-421 (2007); D. F. Woodward, et al., Prostamides (prostaglandin-ethanolamides) and their pharmacology, Br. J. Pharmacol. 153(3): 410-419 (2008); D. F. Woodward, et al., The pharmacology and therapeutic relevance of endocannabinoid derived cyclo-oxygenase (COX)-2 products, Pharmacol. Ther. 120(1): 71-80 (2008), each of which is hereby incorporated by reference in its entirety. For example, prostamides possess their own novel biological activity and have longer half-lives in plasma than prostaglandins, indicating that they may exert actions systemically either as prostaglandin precursors or as unique signal mediators. Hence, prostamides and their prostaglandin-glycerol esters appear to represent a new class of neutral lipid mediators with separate receptors/transduction pathways.

The biosynthesis of prostaglandins, prostamides and prostaglandin-glycerol esters are similar (FIG. 9). The first step of prostaglandin biosynthesis requires the oxidation of arachidonic acid by cyclo-oxygenase-1 (COX-1) and cyclo-oxygenase-2 (COX-2) to produce the endoperoxide intermediates PGG₂ and PGH₂, which are then converted by specific PG synthases to the various prostaglandins. For example, PGD₂ is formed by prostaglandin D synthase-directed isomeration of PGH₂. Similarly, PGH₂ is converted to PGE₂ by prostaglandin E synthase and PGF₂ₐ by prostaglandin F synthase. Moreover, PGD₂ is substrate for prostaglandin F synthase, with the resultant formation of 11β-PGF_{2α}. Lastly, prostacyclin synthase catalyze the formation of PGI₂ from PGH₂. Non-limiting examples of prostaglandins include PGD₂, PGE₂, PGF_{2α}, 11β-3-PGF_{2α}, PGG₂, PGH₂, PGI₂, prostacyclin, and thromboxane A₂.

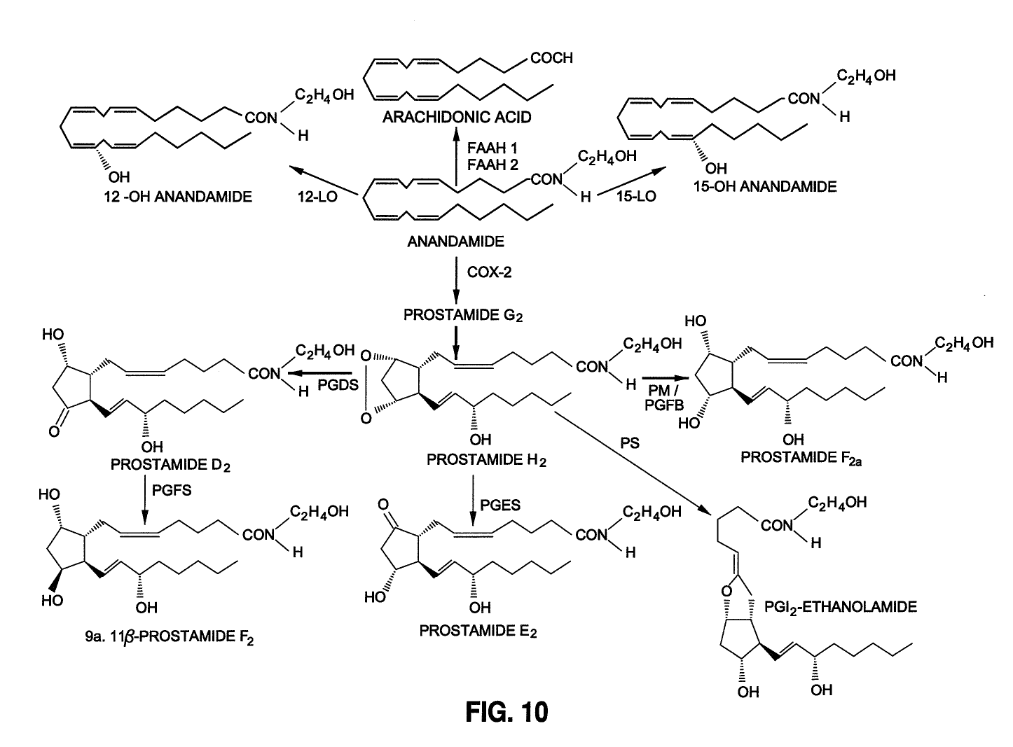

In a similar fashion, arachidonylethanolamide (anandamide) is oxidized to a range of prostamides that closely approaches that of the prostaglandins formed from arachidonic acid (FIGS. 9 and 10). For example, COX-2 oxidizes anandamide to prostamide G₂ and prostamide H₂. Prostamide D₂ is formed by prostaglandin D synthase-directed isomeration of prostamide H₂. Similarly, prostamide H₂ is converted to prostamide E₂ by prostaglandin E synthase and prostamide F_{2α} by prostaglandin F synthase. Moreover, prostamide D₂ is substrate for prostaglandin F synthase, with the resultant formation of 11β-prostamide F_{2α}. Lastly, prostacyclin synthase catalyze the formation of prostamide I₂ from prostamide H₂. As such, non-limiting examples of protamides include prostamide D₂ (PGD₂-ethanolamide), prostamide E₂ (PGE₂-ethanolamide), prostamide F_{2α} (PGF_{2α}-ethanolamide), 11β-prostamide F_{2α} (11β-3-PGF_{2α}-ethanolamide), prostamide G₂ (PGG₂-ethanolamide), prostamide H₂ (PGH₂-ethanolamide), and prostamide I₂ (PGI₂-ethanolamide).

Likewise, 2-arachidonylglycerol (2-AG) is oxidized to a range of prostaglandin-glycerol esters that closely approaches that of the prostaglandins formed from arachidonic acid and prostamide formed from arachidonylethanolamide (FIG. 9). Non-limiting examples of prostaglandin-glycerol esters include PGD₂-glycerol ester, PGE₂-glycerol ester, PGF_{2α}-glycerol ester, 11β-PGF_{2α}-glycerol ester, PGG₂-glycerol ester, PGH₂-glycerol ester, PGI₂-glycerol ester, prostacyclin-glycerol ester, and thromboxane A₂-glycerol ester.

Thus, in one embodiment, a method for treating a condition associated with hair loss, hair thinning, or hair color loss in an individual in need thereof comprises the step of administering a therapeutically effective amount of a composition comprising a prostaglandin, prodrug thereof, salt thereof, or mixtures thereof to the individual, wherein the administration results in a reduction in an attribute associated with hair loss, hair thinning, or hair color loss. In an aspect of this method, a reduction in an attribute associated with hair loss, hair thinning, or hair color loss comprises increasing new hair shaft growth, increasing the rate of hair shaft growth, increasing hair shaft thickness, increasing hair shaft length, increasing hair density, increasing number of hairs shafts produce per hair follicle, increasing penetration of the hair follicle into the dermis, increasing hair shaft pigmentation, increasing hair shaft melanization, increasing hair shaft keratinization, increasing hair luster, converting intermediate or vellus hair to terminal hair, increasing hair health, increasing the time a hair follicle remains in anagen phase, increasing the time a hair follicle remains in catagen phase, or increasing the time a hair follicle remains in telogen phase, prolonging or preventing hair shaft release from the hair follicle, prolonging or preventing hair follicle apoptosis, or prolonging or preventing melanocyte cell death. In another aspect of this embodiment, a prostaglandin is a PGD₂, a PGE₂, a PGF_{2α}, a 11β-PGF_{2α}, a PGG₂, a PGH₂, a PGI₂, a prostacyclin, or a thromboxane A₂.

In another embodiment, a method for treating a condition associated with hair loss, hair thinning, or hair color loss in an individual in need thereof comprises the step of administering a therapeutically effective amount of a composition comprising a prostamide, prodrug thereof, salt thereof, or mixtures thereof to the individual, wherein the administration results in a reduction in an attribute associated with hair loss, hair thinning, or hair color loss. In an aspect of this method, a reduction in an attribute associated with hair loss, hair thinning, or hair color loss comprises increasing new hair shaft growth, increasing the rate of hair shaft growth, increasing hair shaft thickness, increasing hair shaft length, increasing hair density, increasing number of hairs shafts produce per hair follicle, increasing penetration of the hair follicle into the dermis, increasing hair shaft pigmentation, increasing hair shaft melanization, increasing hair shaft keratinization, increasing hair luster, converting intermediate or vellus hair to terminal hair, increasing hair health, increasing the time a hair follicle remains in anagen phase, increasing the time a hair follicle remains in catagen phase, or increasing the time a hair follicle remains in telogen phase, prolonging or preventing hair shaft release from the hair follicle, prolonging or preventing hair follicle apoptosis, or prolonging or preventing melanocyte cell death.

In another aspect of this embodiment, a prostamide is a prostamide D₂ (PGD₂-ethanolamide), a prostamide E₂ (PGE₂-ethanolamide), a prostamide F_{2α} (PGF_{2α}-ethanolamide), a 11β-prostamide F_{2α} (11β-PGF_{2α}-ethanolamide), a prostamide G₂ (PGG₂-ethanolamide), a prostamide H₂ (PGH₂-ethanolamide), or a prostamide I₂ (PGI₂-ethanolamide).

In another embodiment, a method for treating a condition associated with hair loss, hair thinning, or hair color loss in an individual in need thereof comprises the step of administering a therapeutically effective amount of a composition comprising a prostaglandin-glycerol ester, prodrug thereof, salt thereof, or mixtures thereof to the individual, wherein the administration results in a reduction in an attribute associated with hair loss, hair thinning, or hair color loss. In an aspect of this method, a reduction in an attribute associated with hair loss, hair thinning, or hair color loss comprises increasing new hair shaft growth, increasing the rate of hair shaft growth, increasing hair shaft thickness, increasing hair shaft length, increasing hair density, increasing number of hairs shafts produce per hair follicle, increasing penetration of the hair follicle into the dermis, increasing hair shaft pigmentation, increasing hair shaft melanization, increasing hair shaft keratinization, increasing hair luster, converting intermediate or vellus hair to terminal hair, increasing hair health, increasing the time a hair follicle remains in anagen phase, increasing the time a hair follicle remains in catagen phase, or increasing the time a hair follicle remains in telogen phase, prolonging or preventing hair shaft release from the hair follicle, prolonging or preventing hair follicle apoptosis, or prolonging or preventing melanocyte cell death. In another aspect of this embodiment, a prostaglandin-glycerol ester is a PGD₂-glycerol ester, a PGE₂-glycerol ester, a PGF_{2α}-glycerol ester, a 11β-PGF_{2α}-glycerol ester, a PGG₂-glycerol ester, a PGH₂-glycerol ester, a PGI₂-glycerol ester, a prostacyclin-glycerol ester, or a thromboxane A₂-glycerol ester.

In another embodiment, a method for treating a condition associated with hair loss, hair thinning, or hair color loss in an individual in need thereof comprises the step of administering a therapeutically effective amount of a composition comprising prostaglandin analog, prostamide analog, or PG-glycerol ester analog, prodrug thereof, salt thereof, or mixtures thereof to the individual, wherein the administration results in a reduction in an attribute associated with hair loss, hair thinning, or hair color loss. In an aspect of this method, a reduction in an attribute associated with hair loss, hair thinning, or hair color loss comprises increasing new hair shaft growth, increasing the rate of hair shaft growth, increasing hair shaft thickness, increasing hair shaft length, increasing hair density, increasing number of hairs shafts produce per hair follicle, increasing penetration of the hair follicle into the dermis, increasing hair shaft pigmentation, increasing hair shaft melanization, increasing hair shaft keratinization, increasing hair luster, converting intermediate or vellus hair to terminal hair, increasing hair health, increasing the time a hair follicle remains in anagen phase, increasing the time a hair follicle remains in catagen phase, or increasing the time a hair follicle remains in telogen phase, prolonging or preventing hair shaft release from the hair follicle, prolonging or preventing hair follicle apoptosis, or prolonging or preventing melanocyte cell death. In another aspect of this embodiment, a prostaglandin analog, prostamide analog, or PG-glycerol ester analog comprises a compound having the structure:

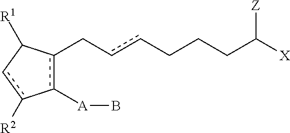

wherein the dashed bonds represent a single or double bond which can be in the cis or trans configuration;

X is —OR⁴ or —N(R⁴)₂ wherein R⁴ is independently selected from hydrogen, a lower alkyl radical having from one to six carbon atoms, CH(OH)₂, ethyl and —H;

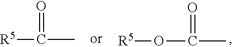

wherein R⁵ is a lower alkyl radical having from one to six carbon atoms;
Z is =0 or represents two hydrogen radicals;
one of R¹ and R² is =0, —OH or a —O(CO)R₆ group, and the other one is —OH or —O(CO)R₆, or R¹ is =0 and R² is hydrogen, wherein R₆ is a saturated or unsaturated acyclic hydrocarbon group having from 1 to about 20 carbon atoms, or —(CH₂)ₘR₇, wherein m is 0 or an integer of from 1 to 10, and R₇ is cycloalkyl radical, having from three to seven carbon atoms, or a hydrocarbyl aryl or heteroaryl radical, as defined above;
A is an alkylene or alkenylene radical having from two to six carbon atoms, which radical may be interrupted by one or more oxide radicals and substituted with one or more hydroxy, oxo, alkyloxy or alkylcarboxy groups wherein the alkyl radical comprises from one to six carbon atoms; and
B is a cycloalkyl radical having from three to seven carbon atoms, an aryl radical, selected from hydrocarbyl aryl and heteroaryl radicals having from four to ten carbon atoms wherein the heteroatom is selected from nitrogen, oxygen and sulfur atoms.

In another aspect of this embodiment, a prostaglandin analog, prostamide analog, or PG-glycerol ester analog is a cyclopentane heptanoic acid, 2-cycloalkyl or arylalkyl compound.

In another aspect of this embodiment, a prostaglandin analog, prostamide analog, or PG-glycerol ester analog comprises a compound having the structure

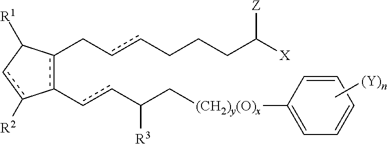

wherein X is —OR⁴ or —N(R⁴)₂ wherein R⁴ is selected from hydrogen, a lower alkyl radical having from one to six carbon atoms, CH(OH)₂,

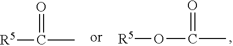

wherein R⁵ is a lower alkyl radical having from one to six carbon atoms;
Z is =0 or represents two hydrogen radicals;
one of R¹ and R² is =0, —OH or a —O(CO)R⁶ group, and the other one is —OH or —O(CO)R⁶, or R¹ is =0 and R² is hydrogen, wherein R⁶ is a saturated or unsaturated acyclic hydrocarbon group having from 1 to about 20 carbon atoms, or —(CH₂)ₘR⁷, wherein m is 0 or an integer of from 1 to 10, and R⁷ is cycloalkyl radical, having from three to seven carbon atoms, or a hydrocarbyl aryl or heteroaryl radical, as defined above;
R³ is =0, —OH or O(CO)R⁶;
y is 0 or 1, x is 0 or 1 and x and y are not both 1; and
Y is selected the group consisting of alkyl, halo, nitro, amino, thiol, hydroxy, alkyloxy, alkylcarboxy and halo substituted alkyl, wherein said alkyl radical comprises from one to six carbon atoms, n is 0 or an integer of from 1 to 3.

In another aspect of this embodiment, a prostaglandin analog, prostamide analog, or PG-glycerol ester analog comprises a compound having the structure:

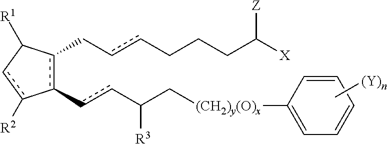

wherein hatched lines indicate the alpha configuration and solid triangles indicate the beta configuration;
X is —OR⁴ or —N(R⁴)₂ wherein R⁴ is selected from hydrogen, a lower alkyl radical having from one to six carbon atoms, CH(OH)₂,

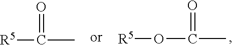

wherein R⁵ is a lower alkyl radical having from one to six carbon atoms;
Z is =0 or represents two hydrogen radicals;
one of R¹ and R² is =0, —OH or a —O(CO)R⁶ group, and the other one is —OH or —O(CO)R⁶, or R¹ is =0 and R² is hydrogen, wherein R⁶ is a saturated or unsaturated acyclic hydrocarbon group having from 1 to about 20 carbon atoms, or —(CH₂)ₘR⁷, wherein m is 0 or an integer of from 1 to 10, and R⁷ is cycloalkyl radical, having from three to seven carbon atoms, or a hydrocarbyl aryl or heteroaryl radical, as defined above;
R³ is =0, —OH or O(CO)R⁶;
y is 0 or 1, x is 0 or 1 and x and y are not both 1; and
Y is selected the group consisting of alkyl, halo, nitro, amino, thiol, hydroxy, alkyloxy, alkylcarboxy and halo substituted alkyl, wherein said alkyl radical comprises from one to six carbon atoms, n is 0 or an integer of from 1 to 3.

In another aspect of this embodiment, a prostaglandin analog, prostamide analog, or PG-glycerol ester analog comprises a compound having the structure:

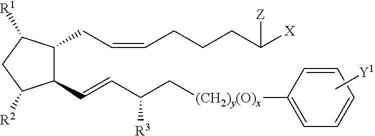

wherein X is —OR⁴ or —N(R⁴)₂ wherein R⁴ is selected from hydrogen, a lower alkyl radical having from one to six carbon atoms, CH(OH)₂,

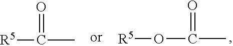

wherein R⁵ is a lower alkyl radical having from one to six carbon atoms;
Z is =0 or represents two hydrogen radicals;
one of R¹ and R² is =0, —OH or a —O(CO)R⁶ group, and the other one is —OH or —O(CO)R⁶, or R¹ is =0 and R² is hydrogen, wherein R⁶ is a saturated or unsaturated acyclic hydrocarbon group having from 1 to about 20 carbon atoms, or —(CH₂)ₘR⁷, wherein m is 0 or an integer of from 1 to 10, and R⁷ is cycloalkyl radical, having from three to seven carbon atoms, or a hydrocarbyl aryl or heteroaryl radical, as defined above;
R³ is =0, —OH or O(CO)R⁶;
y is 0 or 1, x is 0 or 1 and x and y are not both 1; and
Y¹ is Cl or trifluoromethyl.

In another aspect of this embodiment, a prostaglandin analog, prostamide analog, or PG-glycerol ester analog comprises a compound having the structure:

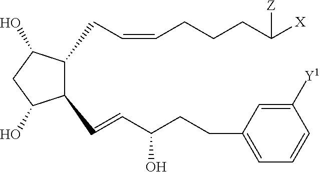

wherein X is —OR⁴ or —N(R⁴)₂ wherein R⁴ is selected from hydrogen, a lower alkyl radical having from one to six carbon atoms, CH(OH)₂,

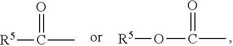

wherein R⁵ is a lower alkyl radical having from one to six carbon atoms;
Z is =0 or represents two hydrogen radicals;
Y¹ is Cl or trifluoromethyl; and
9-, 11- and/or 15 esters thereof.

In another aspect of this embodiment, a prostaglandin analog comprises a compound having the structure:

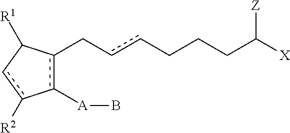

wherein the dashed bonds represent a single or double bond which can be in the cis or trans configuration;
X is —OR⁴, wherein R⁴ is selected from hydrogen, a lower alkyl radical having from one to six carbon atoms,

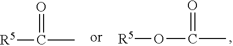

wherein R⁵ is a lower alkyl radical having from one to six carbon atoms;
Z is =0 or represents two hydrogen radicals;
one of R¹ and R² is =0, —OH or a —O(CO)R⁶ group, and the other one is —OH or —O(CO)R⁶, or R¹ is =0 and R² is hydrogen, wherein R⁶ is a saturated or unsaturated acyclic hydrocarbon group having from 1 to about 20 carbon atoms, or —(CH₂)ₘR⁷, wherein m is 0 or an integer of from 1 to 10, and R⁷ is cycloalkyl radical, having from three to seven carbon atoms, or a hydrocarbyl aryl or heteroaryl radical, as defined above;
A is an alkylene or alkenylene radical having from two to six carbon atoms, which radical may be interrupted by one or more oxide radicals and substituted with one or more hydroxy, oxo, alkyloxy or alkylcarboxy groups wherein the alkyl radical comprises from one to six carbon atoms; and
B is a cycloalkyl radical having from three to seven carbon atoms, an aryl radical, selected from hydrocarbyl aryl and heteroaryl radicals having from four to ten carbon atoms wherein the heteroatom is selected from nitrogen, oxygen and sulfur atoms.

In another aspect of this embodiment, a prostaglandin analog comprises a compound having the structure:

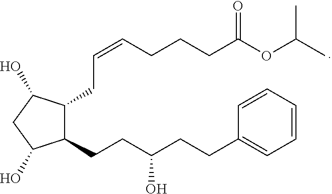 [SMILES: CC(C)OC(=O)CCC/C=C\C[C@@H]1[C@@H](CC[C@@H](O)CCc2ccccc2)[C@H](O)C[C@@H]1O]

This prostaglandin is known as latanoprost and is publicly available in a topical ophthalmic solution under the tradename, XALATAN® by Pfizer Inc., New York, USA.

In another aspect of this embodiment, a prostamide analog comprises a compound having the structure:

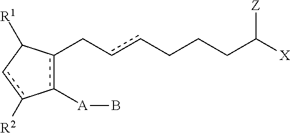

wherein the dashed bonds represent a single or double bond which can be in the cis or trans configuration;
X is —N(R⁴)₂, wherein R⁴ is selected from hydrogen, a lower alkyl radical having from one to six carbon atoms,

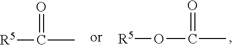

wherein R⁵ is a lower alkyl radical having from one to six carbon atoms;
Z is =0 or represents two hydrogen radicals;
one of R¹ and R² is =0, —OH or a —O(CO)R⁶ group, and the other one is —OH or —O(CO)R⁶, or R¹ is =0 and R² is hydrogen, wherein R⁶ is a saturated or unsaturated acyclic hydrocarbon group having from 1 to about 20 carbon atoms, or —(CH₂)ₘR⁷, wherein m is 0 or an integer of from 1 to 10, and R⁷ is cycloalkyl radical, having from three to seven carbon atoms, or a hydrocarbyl aryl or heteroaryl radical, as defined above;
A is an alkylene or alkenylene radical having from two to six carbon atoms, which radical may be interrupted by one or more oxide radicals and substituted with one or more hydroxy, oxo, alkyloxy or alkylcarboxy groups wherein the alkyl radical comprises from one to six carbon atoms; and
B is a cycloalkyl radical having from three to seven carbon atoms, an aryl radical, selected from hydrocarbyl aryl and heteroaryl radicals having from four to ten carbon atoms wherein the heteroatom is selected from nitrogen, oxygen and sulfur atoms.

In another aspect of this embodiment, a prostamide analog comprises a compound having the structure:

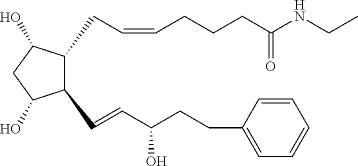 [SMILES: CCNC(=O)CCC/C=C\C[C@@H]1[C@@H](/C=C/[C@@H](O)CCc2ccccc2)[C@H](O)C[C@@H]1O]

This compound is also known as bimatoprost and sold under the tradenames LATISSE® and LUMIGAN® by Allergan, Inc., California, USA.

In another aspect of this embodiment, a PG-glycerol ester analog comprises a compound having the structure:

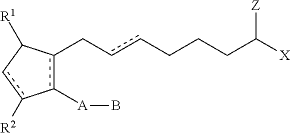

wherein the dashed bonds represent a single or double bond which can be in the cis or trans configuration;
X is —OR⁴, wherein R⁴ is CH(OH)₂;
Z is =0 or represents two hydrogen radicals;
one of R¹ and R² is =0, —OH or a —O(CO)R⁶ group, and the other one is —OH or —O(CO)R⁶, or R¹ is =0 and R² is hydrogen, wherein R⁶ is a saturated or unsaturated acyclic hydrocarbon group having from 1 to about 20 carbon atoms, or —(CH₂)ₘR⁷, wherein m is 0 or an integer of from 1 to 10, and R⁷ is cycloalkyl radical, having from three to seven carbon atoms, or a hydrocarbyl aryl or heteroaryl radical, as defined above;
A is an alkylene or alkenylene radical having from two to six carbon atoms, which radical may be interrupted by one or more oxide radicals and substituted with one or more hydroxy, oxo, alkyloxy or alkylcarboxy groups wherein the alkyl radical comprises from one to six carbon atoms; and
B is a cycloalkyl radical having from three to seven carbon atoms, an aryl radical, selected from hydrocarbyl aryl and heteroaryl radicals having from four to ten carbon atoms wherein the heteroatom is selected from nitrogen, oxygen and sulfur atoms.

In another aspect of this embodiment, a prostaglandin analog, prostamide analog, or PG-glycerol ester analog comprises a compound having the structure listed in Table 1

TABLE 1
Prostaglandin analog, prostamide analog,
or PG-glycerol ester analog

| Com- | |
|---|---|
| pound | Compound Formula |
| 1 | Cyclopentane heptenamide-5-cis-2-(3α-hydroxy-5phenyl- |
| | 1-trans-pentenyl)-3,5-dihydroxy, [1_{α,}2_{β,}3_{α,}5₆α] |
| 2 | Cyclopentane N,N-dimethylheptenamide-5-cis-2-(3α- |
| | hydroxy-5-phenyl-1-trans-pentenyl)-3,5-hydroxy, |
| | [1_{α,}2_{β,}3_{α,}5_{α}] |
| 3 | Cyclopentane heptenylamide-5-cis-2-(3α-hydroxy-4- |
| | metachlorophenoxy-1-trans-pentenyl)-3,5-dihydroxy, |
| | [1_{α,}2_{β,}3_{α,}5_{α}] |
| 4 | Cyclopentane heptenylamide-5-cis-2-(3α-hydroxy-4- |
| | trifluoromethylphenoxy-1-trans-pentenyl)-3,5-dihydroxy, |
| | [1_{α,}2_{β,}3_{α,}5_{α}] |
| 5 | Cyclopentane N-isopropyl heptenamide-5-cis-2-(3α- |
| | hydroxy-5-phenyl-1-trans-pentenyl)-3,5-dihydroxy, |
| | [1_{α,}2_{β,}3_{α,}5_{α}] |
| 6 | Cyclopentane N-ethyl heptenamide-5-cis-2-(3α- |
| | hydroxy-5-phenyl-1-trans-pentenyl)-3,5-dihydroxy, |
| | [1_{α,}2_{β,}3_{α,}5_{α}] |
| 7 | Cyclopentane N-methyl heptenamide-5-cis-2-(3α- |
| | hydroxy-5-phenyl-1-trans-pentenyl)-3,5-dihydroxy, |
| | [1_{α,}2_{β,}3_{α,}5_{α}] |
| 8 | Cyclopentane heptenamide-5-cis-2-(3α-hydroxy- |
| | 4-meta-chlorophenoxy-1-trans-butenyl)-3,5-dihydroxy, |
| | [1_{α,}2_{β,}3_{α,}5_{α}] |

Synthesis of the compounds in Table 1 is disclosed in U.S. Pat. No. 5,607,978, incorporated herein by reference in its entirety.

As used herein, either alone or in combination, the term “alkyloxy” or “alkoxy” refers to a functional group comprising an alkyl ether group. Examples of alkoxys include, without limitation, methoxy, ethoxy, n-propoxy, isopropoxy, n-butoxy, iso-butoxy, sec-butoxy, tert-butoxy, and the like.

As used herein, either alone or in combination, the term “alkyl” refers to a functional group comprising a straight-chain or branched-chain hydrocarbon containing from 1 to 20 carbon atoms linked exclusively by single bonds and not having any cyclic structure. An alkyl group may be optionally substituted as defined herein. Examples of alkyl groups includes, without limitation methyl, ethyl, n-propyl, isopropyl, n-butyl, isobutyl, sec-butyl, tert-butyl, pentyl, iso-amyl, hexyl, heptyl, octyl, noyl, decyl, undecyl, dodecyl tridecyl, tetradecyl, pentadecyl, hexadecyl, heptadecyl, octadecyl, nonadecyl, eicosyl, and the like.

The term “alkylene,” as used herein, alone or in combination, refers to a saturated aliphatic group derived from a straight or branched chain saturated hydrocarbon attached at two or more positions, such as methylene (—O₂—). Unless otherwise specified, the term “alkyl” may include “alkylene” groups.

As used herein, either alone or in combination, the term “alkylcarbonyl” or “alkanoyl” refers to a functional group comprising an alkyl group attached to the parent molecular moiety through a carbonyl group. Examples of alkylcarbonyl groups include, without limitation, methylcarbonyl, ethylcarbonyl, and the like.

As used herein, either alone or in combination, the term “alkynyl” refers to a functional group comprising a straight-chain or branched-chain hydrocarbon containing from 2 to 20 carbon atoms and having one or more carbon-carbon triple bonds and not having any cyclic structure. An alkynyl group may be optionally substituted as defined herein. Examples of alkynyl groups include, without limitation, ethynyl, propynyl, hydroxypropynyl, butynyl, butyn-1-yl, butyn-2-yl, 3-methylbutyn-1-yl, pentynyl, pentyn-1-yl, hexynyl, hexyn-2-yl, heptynyl, octynyl, nonynyl, decynyl, undecynyl, dodecynyl, tridecynyl, tetradecynyl, pentadecynyl, hexadecynyl, heptadecynyl, octadecynyl, nonadecynyl, eicosynyl, and the like.

The term “alkynylene” refers to a carbon-carbon triple bond attached at two positions such as ethynylene (—C:::C—, —C≡C—). Unless otherwise specified, the term “alkynyl” may include “alkynylene” groups.

As used herein, either alone or in combination, the term “aryl”, “hydrocarbyl aryl”, or “aryl hydrocarbon” refers to a functional group comprising a substituted or unsubstituted aromatic hydrocarbon with a conjugated cyclic molecular ring structure of 3 to 12 carbon atoms. An aryl group can be monocyclic, bicyclic or polycyclic, and may optionally include one to three additional ring structures, such as, e.g., a cycloalkyl, a cycloalkenyl, a heterocycloalkyl, a heterocycloalkenyl, or a heteroaryl. The term “aryl” includes, without limitation, phenyl(benzenyl), thiophenyl, indolyl, naphthyl, totyl, xylyl, anthracenyl, phenanthryl, azulenyl, biphenyl, naphthalenyl, 1-methylnaphthalenyl, acenaphthenyl, acenaphthylenyl, anthracenyl, fluorenyl, phenalenyl, phenanthrenyl, benzo[a]anthracenyl, benzo[c]phenanthrenyl, chrysenyl, fluoranthenyl, pyrenyl, tetracenyl(naphthacenyl), triphenylenyl, anthanthrenyl, benzopyrenyl, benzo[a]pyrenyl, benzo[e]fluoranthenyl, benzo[ghi]perylenyl, benzo[j]fluoranthenyl, benzo[k]fluoranthenyl, corannulenyl, coronenyl, dicoronylenyl, helicenyl, heptacenyl, hexacenyl, ovalenyl, pentacenyl, picenyl, perylenyl, and tetraphenylenyl.

As used herein, either alone or in combination, the term “lower aryl” refers to a functional group comprising a substituted or unsubstituted aromatic hydrocarbon with a conjugated cyclic molecular ring structure of 3 to 6 carbon atoms. Examples of lower aryl groups include, without limitation, phenyl and naphthyl.

As used herein, either alone or in combination, the term “carboxyl” or “carboxy” refers to the functional group —C(═O)OH or the corresponding “carboxylate” anion —C(═O)O—. Examples include, without limitation, formic acid, acetic acid, oxalic acid, benzoic acid. An “O-carboxyl” group refers to a carboxyl group having the general formula RCOO, wherein R is an organic moeity or group. A “C-carboxyl” group refers to a carboxyl group having the general formula COOR, wherein R is an organic moeity or group.

As used herein, either alone or in combination, the term “cycloalkyl”, “carbocyclicalkyl”, and “carbocyclealkyl” refers to a functional group comprising a substituted or unsubstituted non-aromatic hydrocarbon with a non-conjugated cyclic molecular ring structure of 3 to 12 carbon atoms linked exclusively with carbon-carbon single bonds in the carbon ring structure. A cycloalkyl group can be monocyclic, bicyclic or polycyclic, and may optionally include one to three additional ring structures, such as, e.g., an aryl, a heteroaryl, a cycloalkenyl, a heterocycloalkyl, or a heterocycloalkenyl.

As used herein, either alone or in combination, the term “lower cycloalkyl” refers to a functional group comprising a monocyclic substituted or unsubstituted non-aromatic hydrocarbon with a non-conjugated cyclic molecular ring structure of 3 to 6 carbon atoms linked exclusively with carbon-carbon single bonds in the carbon ring structure. Examples of lower cycloalkyl groups include, without limitation, cyclopropyl, cyclobutyl, cyclopentyl, and cyclohexyl.

As used herein, the term “functional group” refers to a specific group of atoms within a molecule that are responsible for the characteristic chemical reactions of those molecules.

As used herein, either alone or in combination, the term “heteroalkyl” refers to a functional group comprising a straight-chain or branched-chain hydrocarbon containing from 1 to 20 atoms linked exclusively by single bonds, where at least one atom in the chain is a carbon and at least one atom in the chain is O, S, N, or any combination thereof. The heteroalkyl group can be fully saturated or contain from 1 to 3 degrees of unsaturation. The non-carbon atoms can be at any interior position of the heteroalkyl group, and up to two non-carbon atoms may be consecutive, such as, e.g., —CH₂—NH—OCH₃. In addition, the non-carbon atoms may optionally be oxidized and the nitrogen may optionally be quaternized.

As used herein, either alone or in combination, the term “heteroaryl” refers to a functional group comprising a substituted or unsubstituted aromatic hydrocarbon with a conjugated cyclic molecular ring structure of 3 to 12 atoms, where at least one atom in the ring structure is a carbon and at least one atom in the ring structure is O, S, N, or any combination thereof. A heteroaryl group can be monocyclic, bicyclic or polycyclic, and may optionally include one to three additional ring structures, such as, e.g., an aryl, a cycloalkyl, a cycloalkenyl, a heterocycloalkyl, or a heterocycloalkenyl. Examples of heteroaryl groups include, without limitation, acridinyl, benzidolyl, benzimidazolyl, benzisoxazolyl, benzodioxinyl, dihydrobenzodioxinyl, benzodioxolyl, 1,3-benzodioxolyl, benzofuryl, benzoisoxazolyl, benzopyranyl, benzothiophenyl, benzo[c]thiophenyl, benzotriazolyl, benzoxadiazolyl, benzoxazolyl, benzothiadiazolyl, benzothiazolyl, benzothienyl, carbazolyl, chromonyl, cinnolinyl, dihydrocinnolinyl, coumarinyl, dibenzofuranyl, furopyridinyl, furyl, indolizinyl, indolyl, dihydroindolyl, imidazolyl, indazolyl, isobenzofuryl, isoindolyl, isoindolinyl, dihydroisoindolyl, isoquinolyl, dihydroisoquinolinyl, isoxazolyl, isothiazolyl, oxazolyl, oxadiazolyl, phenanthrolinyl, phenanthridinyl, purinyl, pyranyl, pyrazinyl, pyrazolyl, pyridyl, pyrimidinyl, pyridazinyl, pyrrolinyl, pyrrolyl, pyrrolopyridinyl, quinolyl, quinoxalinyl, quinazolinyl, tetrahydroquinolinyl, tetrazolopyridazinyl, tetrahydroisoquinolinyl, thiophenyl, thiazolyl, thiadiazolyl, thienopyridinyl, thienyl, thiophenyl, triazolyl, xanthenyl, and the like.

As used herein, either alone or in combination, the term “lower heteroaryl” refers to a functional group comprising a monocyclic or bycyclic, substituted or unsubstituted aromatic hydrocarbon with a conjugated cyclic molecular ring structure of 3 to 6 atoms, where at least one atom in the ring structure is a carbon and at least one atom in the ring structure is O, S, N, or any combination thereof.

As used herein, either alone or in combination, the term “hydroxy” refers to the functional group hydroxyl (—OH).

As used herein, either alone or in combination, the term “oxo” refers to the functional group ═O.

Pharmaceutically acceptable acid addition salts of the compounds described are those formed from acids which form non-toxic addition salts containing pharmaceutically acceptable anions, such as the hydrochloride, hydrobromide, hydroiodide, sulfate, or bisulfate, phosphate or acid phosphate, acetate, maleate, fumarate, oxalate, lactate, tartrate, citrate, gluconate, saccharate and p-toluene sulphonate salts.

Aspects of the present specification disclose, in part, a composition comprising a prostaglandin, prostamide, prostaglandin-glycerol ester, or analog thereof as disclosed in the present specification. A composition comprising a prostaglandin, prostamide, prostaglandin-glycerol ester, or analog thereof as disclosed in the present specification is generally administered to an individual as a pharmaceutical composition. As used herein, the term “pharmaceutical composition” and refers to a therapeutically effective concentration of an active ingredient, such as, e.g., any of the prostaglandins, prostamides, prostaglandin-glycerol esters, or analogs thereof disclosed in the present specification. Preferably, the pharmaceutical composition comprising an active ingredient does not produce an adverse, allergic, or other untoward or unwanted reaction when administered to an individual. A pharmaceutical composition comprising a prostaglandin, prostamide, prostaglandin-glycerol ester, or analog thereof as disclosed in the present specification is useful for medical and veterinary applications. A pharmaceutical composition may be administered to an individual alone, or in combination with other supplementary active compounds, agents, drugs or hormones. The pharmaceutical compositions may be manufactured using any of a variety of processes, including, without limitation, conventional mixing, dissolving, granulating, dragee-making, levigating, emulsifying, encapsulating, entrapping, and lyophilizing. The pharmaceutical composition can take any of a variety of forms including, without limitation, a sterile solution, suspension, emulsion, lyophilizate, cream, ointment, salve, tablet, pill, pellet, capsule, powder, syrup, elixir, or any other dosage form suitable for administration.

A pharmaceutical composition comprising a prostaglandin, prostamide, prostaglandin-glycerol ester, or analog thereof as disclosed in the present specification can optionally include a pharmaceutically acceptable carrier that facilitates processing of an active ingredient into pharmaceutically acceptable compositions. As used herein, the term “pharmacologically acceptable carrier” is synonymous with “pharmacological carrier” and refers to any carrier that has substantially no long term or permanent detrimental effect when administered and encompasses terms such as “pharmacologically acceptable vehicle, stabilizer, diluent, additive, auxiliary, or excipient.” Such a carrier generally is mixed with an active compound or permitted to dilute or enclose the active compound and can be a solid, semi-solid, or liquid agent. It is understood that the active ingredients can be soluble or can be delivered as a suspension in the desired carrier or diluent. Any of a variety of pharmaceutically acceptable carriers can be used including, without limitation, aqueous media such as, e.g., water, saline, glycine, hyaluronic acid and the like; solid carriers such as, e.g., starch, magnesium stearate, mannitol, sodium saccharin, talcum, cellulose, glucose, sucrose, lactose, trehalose, magnesium carbonate, and the like; solvents; dispersion media; coatings; antibacterial and antifungal agents; isotonic and absorption delaying agents; or any other inactive ingredient. Selection of a pharmacologically acceptable carrier can depend on the mode of administration. Except insofar as any pharmacologically acceptable carrier is incompatible with the active ingredient, its use in pharmaceutically acceptable compositions is contemplated. Non-limiting examples of specific uses of such pharmaceutical carriers can be found in Pharmaceutical Dosage Forms and Drug Delivery Systems (Howard C. Ansel et al., eds., Lippincott Williams & Wilkins Publishers, 7^{th} ed. 1999); Remington: The Science and Practice of Pharmacy (Alfonso R. Gennaro ed., Lippincott, Williams & Wilkins, 20^{th} ed. 2000); Goodman & Gilman's The Pharmacological Basis of Therapeutics (Joel G. Hardman et al., eds., McGraw-Hill Professional, 10^{th} ed. 2001); and Handbook of Pharmaceutical Excipients (Raymond C. Rowe et al., APhA Publications, 4^{th} edition 2003). These protocols are routine and any modifications are well within the scope of one skilled in the art and from the teaching herein.

A pharmaceutical composition disclosed in the present specification can optionally include, without limitation, other pharmaceutically acceptable components (or pharmaceutical components), including, without limitation, buffers, preservatives, tonicity adjusters, salts, antioxidants, osmolality adjusting agents, physiological substances, pharmacological substances, bulking agents, emulsifying agents, wetting agents, sweetening or flavoring agents, and the like. Various buffers and means for adjusting pH can be used to prepare a pharmaceutical composition disclosed in the present specification, provided that the resulting preparation is pharmaceutically acceptable. Such buffers include, without limitation, acetate buffers, borate buffers, citrate buffers, phosphate buffers, neutral buffered saline, and phosphate buffered saline. It is understood that acids or bases can be used to adjust the pH of a composition as needed. Pharmaceutically acceptable antioxidants include, without limitation, sodium metabisulfite, sodium thiosulfate, acetylcysteine, butylated hydroxyanisole, and butylated hydroxytoluene. Useful preservatives include, without limitation, benzalkonium chloride, chlorobutanol, thimerosal, phenylmercuric acetate, phenylmercuric nitrate, a stabilized oxy chloro composition, such as, e.g., PURITE® and chelants, such as, e.g., DTPA or DTPA-bisamide, calcium DTPA, and CaNaDTPA-bisamide. Tonicity adjustors useful in a pharmaceutical composition include, without limitation, salts such as, e.g., sodium chloride, potassium chloride, mannitol or glycerin and other pharmaceutically acceptable tonicity adjustor. The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. It is understood that these and other substances known in the art of pharmacology can be included in a pharmaceutical composition useful in the invention.

Aspects of the present invention provide, in part, administering a therapeutically effective amount of a composition comprising a prostaglandin, prostamide, prostaglandin-glycerol ester, or analog thereof as disclosed in the present specification. As used herein, the term “therapeutically effective amount” is synonymous with “therapeutically effective dose” and when used in reference to treating a condition disclosed in the present specification refers to the minimum dose of a prostaglandin, prostamide, prostaglandin-glycerol ester, or analog thereof as disclosed in the present specification necessary to achieve the desired therapeutic effect and includes a dose sufficient to reduce a symptom or attribute associated with the condition. In aspects of this embodiment, a therapeutically effective amount of a composition comprising a prostaglandin, prostamide, prostaglandin-glycerol ester, or analog thereof as disclosed in the present specification reduces a symptom or attribute associated with a condition by, e.g., at least 10%, at least 20%, at least 30%, at least 40%, at least 50%, at least 60%, at least 70%, at least 80%, at least 90% or at least 100%. In other aspects of this embodiment, a therapeutically effective amount of a composition comprising a prostaglandin, prostamide, prostaglandin-glycerol ester, or analog thereof as disclosed in the present specification reduces a symptom or attribute associated with a condition by, e.g., at most 10%, at most 20%, at most 30%, at most 40%, at most 50%, at most 60%, at most 70%, at most 80%, at most 90% or at most 100%. In yet other aspects of this embodiment, a therapeutically effective amount of a composition comprising a prostaglandin, prostamide, prostaglandin-glycerol ester, or analog thereof as disclosed in the present specification reduces a symptom or attribute associated with a condition by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%. As used herein, the term “about” when qualifying a value of a stated item, number, percentage, or term refers to a range of plus or minus ten percent of the value of the stated item, percentage, parameter, or term. In still other aspects of this embodiment, a therapeutically effective amount of a prostaglandin, prostamide, prostaglandin-glycerol ester, or analog thereof as disclosed in the present specification is the dosage sufficient to achieve the desired therapeutic effect for, e.g., at least one week, at least one month, at least two months, at least three months, at least four months, at least five months, at least six months, at least seven months, at least eight months, at least nine months, at least ten months, at least eleven months, or at least twelve months.

The actual therapeutically effective amount of a composition comprising achieve the desired therapeutic effect to be administered to an individual can be determined by a person of ordinary skill in the art by taking into account factors, including, without limitation, the type of condition, the location of the condition, the cause of the condition, the severity of the condition, the degree of relief desired, the duration of relief desired, the particular prostaglandin, prostamide, prostaglandin-glycerol ester, or analog thereof used, the rate of excretion of the prostaglandin, prostamide, prostaglandin-glycerol ester, or analog thereof used, the pharmacodynamics of the prostaglandin, prostamide, prostaglandin-glycerol ester, or analog thereof used, the nature of the other compounds to be included in the composition, the particular route of administration, the particular characteristics, history and risk factors of the individual, such as, e.g., age, weight, general health and the like, or any combination thereof. Additionally, where repeated administration of a composition comprising a prostaglandin, prostamide, prostaglandin-glycerol ester, or analog thereof is used, the actual effect amount of a composition comprising prostaglandin, prostamide, prostaglandin-glycerol ester, or analog thereof will further depend upon factors, including, without limitation, the frequency of administration, the half-life of the composition comprising prostaglandin, prostamide, prostaglandin-glycerol ester, or analog thereof, or any combination thereof. In is known by a person of ordinary skill in the art that an effective amount of a composition comprising a prostaglandin, prostamide, prostaglandin-glycerol ester, or analog thereof can be extrapolated from in vitro assays and in vivo administration studies using animal models prior to administration to humans. Wide variations in the necessary effective amount are to be expected in view of the differing efficiencies of the various routes of administration. For instance, oral administration generally would be expected to require higher dosage levels than administration by intravenous or intravitreal injection. Variations in these dosage levels can be adjusted using standard empirical routines of optimization, which are well-known to a person of ordinary skill in the art. The precise therapeutically effective dosage levels and patterns are preferably determined by the attending physician in consideration of the above-identified factors.

As a non-limiting example, when administering a composition comprising a prostaglandin, prostamide, prostaglandin-glycerol ester, or analog thereof disclosed in the present specification to an individual, a therapeutically effective amount generally is in the range of about 0.0000001% to about 50% by weight of the total composition. In aspects of this embodiment, an effective amount of a composition comprising a prostaglandin, prostamide, prostaglandin-glycerol ester, or analog thereof disclosed in the present specification can range from, e.g., from about 0.01% to about 5% by weight of the total composition, about 0.01% to about 0.5% by weight of the total composition, or about 0.01% to about 0.05% by weight of the total composition. In yet aspects of this embodiment, an effective amount of a composition comprising a prostaglandin, prostamide, prostaglandin-glycerol ester, or analog thereof disclosed in the present specification can range from, e.g., about 0.01% to about 3% by weight of the total composition, about 0.01% to about 0.3% by weight of the total composition, or about 0.01% to about 0.03% by weight of the total composition. In still aspects of this embodiment, an effective amount of a composition comprising a prostaglandin, prostamide, prostaglandin-glycerol ester, or analog thereof disclosed in the present specification can range from, e.g., about 0.001% to about 30% by weight of the total composition, about 0.001% to about 3% by weight of the total composition, about 0.001% to about 0.3% by weight of the total composition, or about 0.001% to about 0.03% by weight of the total composition. In further aspects of this embodiment, an effective amount of a composition comprising a prostaglandin, prostamide, prostaglandin-glycerol ester, or analog thereof disclosed in the present specification can range from, e.g., about 0.01% to about 2% by weight of the total composition, about 0.01% to about 0.2% by weight of the total composition, or about 0.01% to about 0.02% by weight of the total composition. In yet further aspects of this embodiment, an effective amount of a composition comprising a prostaglandin, prostamide, prostaglandin-glycerol ester, or analog thereof disclosed in the present specification can range from, e.g., about 0.001% to about 20% by weight of the total composition, about 0.001% to about 2% by weight of the total composition, about 0.001% to about 0.2% by weight of the total composition, or about 0.001% to about 0.02% by weight of the total composition. In still further aspects of this embodiment, an effective amount of a composition comprising a prostaglandin, prostamide, prostaglandin-glycerol ester, or analog thereof disclosed in the present specification can range from, e.g., about 0.03% by weight of the total composition. In other embodiments, the effective amount is about 0.02% by weight of the total composition.

In another embodiment, two or more prostaglandins, prostamides, prostaglandin-glycerol esters, analogs thereof, or any combination thereof disclosed in the present specification can be used in a single composition. The combinations can be used in ratios that attain a desired effect. The total load in a composition will generally range from about 0.0000001% to about 50% by weight of the total composition. In certain embodiments, the effective amount will range from about 0.001% to about 30% by weight of the total composition. In other embodiments, the effective amount will range from about 0.01% to about 5% by weight of the total composition. In certain embodiments, the effective amount is about 0.03% by weight of the total composition.

Thus, in an embodiment, a composition comprises a prostaglandin, prostamide, prostaglandin-glycerol ester, or analog thereof as disclosed in the present specification. In an aspect of this embodiment, a pharmaceutical composition comprises a prostaglandin, prostamide, prostaglandin-glycerol ester, or analog thereof as disclosed in the present specification and a pharmacological carrier. In another aspect of this embodiment, a pharmaceutical composition comprises a prostaglandin, prostamide, prostaglandin-glycerol ester, or analog thereof as disclosed in the present specification and a pharmacological component. In yet another aspect of this embodiment, a pharmaceutical composition comprises a prostaglandin, prostamide, prostaglandin-glycerol ester, or analog thereof as disclosed in the present specification, a pharmacological carrier, and a pharmacological component. In other aspects of this embodiment, a pharmaceutical composition comprises a prostaglandin, prostamide, prostaglandin-glycerol ester, or analog thereof as disclosed in the present specification and at least one pharmacological carrier, at least one pharmaceutical component, or at least one pharmacological carrier and at least one pharmaceutical component.

Hair is a filamentous outgrowth of protein found only on mammals, including humans. The total number of hair follicles for an adult human is estimated at 5 million with 1 million on the head of which 100,000 alone cover the scalp. In humans, the only external regions of skin devoid of hair follicles are the palms of the hands and soles of the feet. The hair of non-human mammal species is commonly referred to as fur. In some species, hair is absent at certain stages of life.

The basic hair structure remains essentially the same throughout the range of mammalian species with modifications for specialized functions. Each hair comprises two structures: the hair shaft and the hair follicle. THE MOLECULAR AND STRUCTURAL BIOLOGY OF HAIR (eds. K. S. Stenn, A. G. Messenger, and H. P. Baden H P; Ann NY Acad Sci; New York: New York Academy of Sciences, 1991). Located in the dermal layer of the skin, the hair follicle can be recognized as a separate entity within the skin with formation and maintenance based on interaction between dermal and epidermal components.

The hair shaft is primary composed of keratin that is organized into three layers called the medulla, cortex and cuticle. The medulla is the inner layer is not necessarily present in all hair types. The next keratin layer is the cortex, the intermediate layer that makes up the majority of the hair shaft. The outer layer is the cuticle, which is formed by tightly packed scales in an overlapping structure similar to roof shingles and is continuous with the root sheath. Most hair conditioning products attempt to affect the cuticle. Pigment cells, or melanocytes, are distributed throughout the cortex and medulla giving the hair its characteristic color.

The follicle comprises several components. At the base of the follicle is a projection called a dermal papilla, which contains capillaries that supply nutrients to the portion of the follicle called the bulb. The bulb can be divided into two regions: a lower region of undifferentiated cells, and an upper region of actively proliferating cells, called matrix cells, that differentiated to form the inner sheath and the hair shaft. Matrix cells are actively proliferating cells which differentiate and become keratinized to form the hair shaft. During epidermal cell differentiation (anagen phase), matrix cells divide every 23 to 72 hours, faster than any other cells in the body. Matrix cells located in the immediate vicinity of the dermal papilla differentiate and become keratinized to form the hair shaft, whereas matrix cells towards the periphery of a hair follicle proliferate and produce the inner root sheath. The follicle also contains two epidermal layers termed the inner root sheath and outer root sheath. These sheaths protect and mold the growing hair shaft. The inner root sheath can be divided into three layers (cuticle, Huxley layer, and Henle layer) based on structure, patterns of keratinization, and incorporation of trichohyalin. The inner sheath follows the hair shaft until it ends just below the level of a sebaceous gland to leave only the hair shaft to protrude above the epidermis. The outer root sheath continues all the way up to the gland and is distinct from other epidermal components of the hair follicle in that it is continuous with the epidermis. The sebaceous gland produces sebum, a natural oil that conditions the hair shaft and sometimes an apocrine (scent) gland. An erector pili muscle attaches below the gland to a fibrous layer around the outer sheath. The contraction of the muscle pulls on both the hair to make it erect and pulls on the skin making a bumpy surface. Hair color is caused by a pigment (melanin) that is produced by the hair follicle.

Under normal circumstances hair growth in each hair follicle occurs in a cycle that can comprise four main phases: anagen (growth phase), catagen (regression phase), telogen (resting phase), and exogen (shedding phase). K. S. Stenn and R. Paus, Controls of Hair Follicle Cycling, Physiol. Rev. 81: 449-494 (2001); C. A. Higgins, et al., From Telogen to Exogen: Mechanisms Underlying Formation and Subsequent Loss of the Hair Club Fiber, J. Invest. Dermatol. 129: 2100-2109 (2009), each of which is hereby incorporated by reference in its entirety. The anagen phase is the active growth phase of the hair during which the matrix cells in the root of the hair are dividing rapidly. About 80-90% of all hairs are in this phase at any time. Anagen hairs are anchored deeply into the subcutaneous fat and cannot be pulled out easily. When a new hair is formed, it pushes the club hair up the follicle and eventually out. During this phase the hair grows about 0.35 mm a day (1 cm every 28 days), but this rate varies depending on the site of the hair follicle and the age and sex of the individual. Human scalp hair stays in the active anagen phase of growth for 2-6 years, as compared to other sites like on the leg (which stays in the anagen phase for 19 to 26 weeks), on the arm (from 6 to 12 weeks), and in the mustache area, eyelashes, and eyebrows (from 4 to 14 weeks). Human subjects that have difficulty growing their hair beyond a certain length have a short active phase of growth. Human subjects that have very long hair have a long active phase of growth.

Anagen may be divided into six stages: Stage 1-growth of the dermal papilla and on-set of mitotic activity in the germ-like overlying epithelium; Stage 11-bulb matrix cells envelop the dermal papilla and begin differentiation, evolving bulb begins descent along the fibrous streamer; Stage III-bulb matrix cells show differentiation into all follicular components; Stage 1V-matrix melanocytes reactivate; Stage V-hair shaft emerges and dislodges telogen hair; and Stage VI-new hair shaft emerges from skin surface

Anagen I is the period when the cells of the dermal papilla increase in size and show increased RNA synthesis. At the same time, germ cells at the base of the sac undergo vigorous mitotic activity. In Anagen II, the lower part of the follicle grows down into the dermis and partially encloses the dermal papilla. In the matrix ring that surrounds the dermal papilla, differentiation of cells commences and represents the various layers of the hair and the inner root sheath. Anagen III is marked by continued mitotic activity in the external sheath and particularly the ‘germ’ region, and proliferation of the hair follicular melanocytes. At this time, the follicle attains its maximum length, which is about three times the length that it has in the resting condition. The bulb is now completely formed and the papilla cavity is constricted at its base. The melanocytes (epidermal cells capable of synthesizing melanin) become aligned along the papilla cavity and each develops melanin granules and numerous dendritic processes. The internal sheath is now an elongated cone, which extends up to the capsule and club of the old hair. In stage four of anagen, the melanocytes that line the papilla develop dendrites and begin to form melanin (pigment). Although the hair has formed, it is still within the cone of the internal root sheath, which now extends upward to about the level of the sebaceous gland. The keratogenous zone becomes established just below the level of the sebaceous duct. The cuticles of the hair and of the internal sheath are clearly visible. In the upper part of the bulb, a cone of cells, which will become cortex and medulla of the hair, contains pigment granules. The papilla cavity is long and narrow. By Anagen V, the tip of the hair has broken through the tip of the internal sheath, through the intersection of the capsule with the external sheath, and has grown to about the level of the epidermis. The bulb attains its final characteristic shape, which in some hairs is somewhat laterally compressed, in others rounded and symmetrical. Anagen VI begins as soon as the hair emerges at the skin surface and continues until the onset of catagen. In this stage, the hair emerges from the cone of the external root sheath and forces its way to the surface along the original hair shaft, which gets pushed aside, and eventually the clubbed hair is discharged. In the mouse, anagen VI lasts for about 8 days and the hair will grow at the rate of nearly 1 mm/day. In human beings, a follicle on the scalp may remain in this stage for two or more years, producing a hair at the rate of about one-half mm/day.

The catagen phase is a short transitional phase between the anagen and telogen phases which lasts only about 7-21 days. Although brief, this phase can be divided into eight subphases starting with late anagen and ending in early telogen. About 1-3% of all hairs are in this phase at any time. It is a period of controlled regression in which the hair follicle regresses and dismantles the hair growing part of the hair follicle, in part, through apoptosis. During this phase there is involution of the hair follicle and a fundamental restructuring of the extracellular matrix by 1) a withdrawal of dermal papilla and stoppage of hair growth, 2) cessation of matrix cell proliferation and melanocyte melanin synthesis, 3) shrinkage of the outer root sheath and attachment to the hair shaft, 4) movement of the lower hair follicle to the level of the arrector pili muscle, 5) movement of the dermal papilla upward through the skin, coming to rest beneath the hair-follicle bulge, and 6) cessation of protein and pigment production through programmed cell death keratinocyte and melanocytes. Also, there is massive apotosis in the bulbar, transient, portion of the hair follicle contributes to regression of the hair follicle and the formation of a fibrous streamer in the skin. The onset of these apoptotic events seems to be predetermined and finely orchestrated, and as such the events in this phase can be more appropriately described by the term, “programmed cell death”.

The third phase is the telogen phase which, for all practical purposes, can be denominated a “resting phase.” About 10-15% of all hairs are in this phase at any time. During this phase the hair follicle is stops dividing and the hair shaft ceases to grow, the telogen hair completes differentiation, and the last hair growth cells cluster together at the base of the hair shaft to form a club-structure comprising a centrally lying brush of keratinized cells surrounded by apparent mooring cells containing easily found, discrete nuclei and abundant cytoplasm. Called a club hair, the cluster of cells actually holds the hair shaft in the tube of hair follicle. In the final aspect of the telogen phase, a chemical signal causes matrix cells to initiate growth of a new hair shaft from the same hair follicle and the cycle starts over with a new anagen phase. Even though a telogen hair is located near the surface of the skin, it remains firmly anchored to the hair follicle. A telogen hair will eventually be shed in the exogen phase and replaced by the next budding anagen hair. About 30-90 days elapse before telogen hair from the scalp is shed and a new one begins to grow. The time period is much longer for hairs on the eyebrow, eyelash, arm and leg.

The final phase is the exogen, a phase marked by a highly controlled, active process where a telogen hair is actually shed from the follicle. The shed exogen hair has a shrunken base that is more elongated in shape and has a scalloped and pitted margin. Within this shaft base there is little associated cytoplasm and very few shrunken and fragmented nuclei. It is believed that the shrinkage of the hair club and disappears of the brush mooring allows the exogen hair to be shed from the follicle. Normally about 25-150 exogen hairs are shed each day.

Recently, an addition phase called kenogen has been proposed. This phase describes the interval of the hair cycle in which the hair follicle remains empty after the telogen hair has been extruded, but before a new anagen hair reappears.

The prostaglandins, prostamides, prostaglandin-glycerol esters, and analogs thereof disclosed in the present specification can treat a condition associated with hair loss, hair thinning, or hair color loss by stimulating the production of new hair follicles. Without wishing to be bound by any particular theory, it is thought that the disclosed prostaglandins, prostamides, prostaglandin-glycerol esters, and analogs thereof stimulates hair growth and/or increases the amount of time each hair shaft resides in a given follicle. This can be done, e.g., without limitation, by inducing a hair follicle into the anagen phase and/or stimulating the matrix cells to form a new hair shaft, by prolonging the time period a hair follicle remains in anagen phase thereby enabling the follicle to produce a longer and/or thicker hair shaft, by preventing the follicle to enter the exogen phase thereby stopping the release of the hair shaft, or by stopping hair shaft release and also stimulating new hair shaft growth thereby allowing the production of two or more hair shafts per follicle.

Thus, in an embodiment, an attribute associated with hair thinning in an individual is treated by reducing the attribute associated with hair thinning. In aspects of this embodiment, the treatment reduces the attribute associated with hair thinning by increasing the rate of hair shaft growth, increasing hair shaft thickness, increasing hair shaft length, increasing hair density, increasing number of hair shafts produce per follicle, increasing hair shaft pigmentation, increasing hair shaft luster, increasing hair health, increasing the time a hair follicle remains in anagen phase, increasing the time a hair follicle remains in catagen phase, increasing the time a hair follicle remains in telogen phase, or preventing or prolonging the release of a hair shaft from a hair follicle during the exogen phase, preventing or prolonging the initiation of apoptosis in a hair follicle.

In another embodiment, a method of stimulating new hair shaft growth in an epidermal region of an individual comprises the step of administering a therapeutically effective amount of a composition comprising a prostaglandin, a prostamide, a prostaglandin-glycerol ester, or analog thereof as disclosed in the present specification to the individual, wherein the administration results in an increase of new hair shaft growth. In aspects of this embodiment, the increased of new hair growth from a treated epidermal region relative to an untreated epidermal region is, e.g., about 5% greater, about 10% greater, about 15% greater, about 20% greater, about 25% greater, about 30% greater, about 40% greater, about 50% greater, about 60% greater, about 70% greater, about 80% greater, about 90% greater, or about 100% greater. In other aspects of this embodiment, the increased of new hair growth from a treated epidermal region relative to an untreated epidermal region is, e.g., at least 5% greater, at least 10% greater, at least 15% greater, at least 20% greater, at least 25% greater, at least 30% greater, at least 40% greater, at least 50% greater, at least 60% greater, at least 70% greater, at least 80% greater, at least 90%, greater or at least 100% greater.

In another embodiment, a method of increasing the rate of hair shaft growth in an epidermal region of an individual comprises the step of administering a therapeutically effective amount of a composition comprising a prostaglandin, a prostamide, a prostaglandin-glycerol ester, or analog thereof as disclosed in the present specification to the individual, wherein the administration results in an increased the rate of hair shaft growth. In aspects of this embodiment, the increased rate of hair shaft growth from a treated epidermal region relative to an untreated epidermal region is, e.g., about 5% greater, about 10% greater, about 15% greater, about 20% greater, about 25% greater, about 30% greater, about 40% greater, about 50% greater, about 60% greater, about 70% greater, about 80% greater, about 90% greater, or about 100% greater. In other aspects of this embodiment, the increased rate of hair shaft growth from a treated epidermal region relative to an untreated epidermal region is, e.g., at least 5% greater, at least 10% greater, at least 15% greater, at least 20% greater, at least 25% greater, at least 30% greater, at least 40% greater, at least 50% greater, at least 60% greater, at least 70% greater, at least 80% greater, at least 90%, greater or at least 100% greater.

In another embodiment, a method of increasing hair shaft thickness in an epidermal region of an individual comprises the step of administering a therapeutically effective amount of a composition comprising a prostaglandin, a prostamide, a prostaglandin-glycerol ester, or analog thereof as disclosed in the present specification to the individual, wherein the administration results in an increased hair shaft thickness. In aspects of this embodiment, the increased hair shaft thickness from a treated epidermal region relative to an untreated epidermal region is, e.g., about 5% greater, about 10% greater, about 15% greater, about 20% greater, about 25% greater, about 30% greater, about 40% greater, about 50% greater, about 60% greater, about 70% greater, about 80% greater, about 90% greater, or about 100% greater. In other aspects of this embodiment, the increased hair shaft thickness from a treated epidermal region relative to an untreated epidermal region is, e.g., at least 5% greater, at least 10% greater, at least 15% greater, at least 20% greater, at least 25% greater, at least 30% greater, at least 40% greater, at least 50% greater, at least 60% greater, at least 70% greater, at least 80% greater, at least 90%, greater or at least 100% greater. In yet other aspects of this embodiment, the increased hair shaft thickness from a treated epidermal region relative to an untreated epidermal region is, e.g., about 1 μm² to about 1 mm², about 10 μm² to about 1 mm², about 100 μm² to about 1 mm², or about 100 μm² to about 2 mm². Is still other aspects of this embodiment, the increased hair shaft thickness from a treated epidermal region relative to an untreated epidermal region is, e.g., about 100 μm², about 200 μm², about 300 μm², about 400 μm², about 500 μm², about 600 μm², about 700 μm², about 800 μm², about 900 μm², 1 mm², 2 mm², or 3 mm².

In another embodiment, a method of increasing hair shaft length in an epidermal region of an individual comprises the step of administering a therapeutically effective amount of a composition comprising a prostaglandin, a prostamide, a prostaglandin-glycerol ester, or analog thereof as disclosed in the present specification to the individual, wherein the administration results in an increased hair shaft length. In aspects of this embodiment, the increased hair shaft length from a treated epidermal region relative to an untreated epidermal region is, e.g., about 5% greater, about 10% greater, about 15% greater, about 20% greater, about 25% greater, about 30% greater, about 40% greater, about 50% greater, about 60% greater, about 70% greater, about 80% greater, about 90% greater, or about 100% greater. In other aspects of this embodiment, the increased hair shaft length from a treated epidermal region relative to an untreated epidermal region is, e.g., at least 5% greater, at least 10% greater, at least 15% greater, at least 20% greater, at least 25% greater, at least 30% greater, at least 40% greater, at least 50% greater, at least 60% greater, at least 70% greater, at least 80% greater, at least 90%, greater or at least 100% greater. In yet other aspects of this embodiment, the increased hair shaft length from a treated epidermal region relative to an untreated epidermal region is, e.g., about 1 mm to about 500 mm, about 10 mm to about 500 mm, or about 100 mm to about 500 mm. Is still other aspects of this embodiment, the increased hair shaft length from a treated epidermal region relative to an untreated epidermal region is, e.g., about 1 mm, about, 2 mm, about, 3 mm, about 4 mm, about 5 mm, about 6 mm, about 7 mm, about 8 mm, about 9 mm, or about 10 mm.

In another embodiment, a method of increasing hair density in an epidermal region of an individual comprises the step of administering a therapeutically effective amount of a composition comprising a prostaglandin, a prostamide, a prostaglandin-glycerol ester, or analog thereof as disclosed in the present specification to the individual, wherein the administration results in an increased hair density. In aspects of this embodiment, the increased hair density from a treated epidermal region relative to an untreated epidermal region is, e.g., about 5% greater, about 10% greater, about 15% greater, about 20% greater, about 25% greater, about 30% greater, about 40% greater, about 50% greater, about 60% greater, about 70% greater, about 80% greater, about 90% greater, or about 100% greater. In other aspects of this embodiment, the increased hair density from a treated epidermal region relative to an untreated epidermal region is, e.g., at least 5% greater, at least 10% greater, at least 15% greater, at least 20% greater, at least 25% greater, at least 30% greater, at least 40% greater, at least 50% greater, at least 60% greater, at least 70% greater, at least 80% greater, at least 90%, greater or at least 100% greater.

In another embodiment, a method of increasing the number of hair shafts produce per hair follicle in an epidermal region of an individual comprises the step of administering a therapeutically effective amount of a composition comprising a prostaglandin, a prostamide, a prostaglandin-glycerol ester, or analog thereof as disclosed in the present specification to the individual, wherein the administration results in an increased number of hair shafts produce per hair follicle. In aspects of this embodiment, the number of hair follicles producing two or more hair shafts/follicle from a treated epidermal region relative to an untreated epidermal region is, e.g., about 5% greater, about 10% greater, about 15% greater, about 20% greater, about 25% greater, about 30% greater, about 40% greater, about 50% greater, about 60% greater, about 70% greater, about 80% greater, about 90% greater, or about 100% greater. In other aspects of this embodiment, the increased hair shaft length from a treated epidermal region relative to an untreated epidermal region is, e.g., at least 5% greater, at least 10% greater, at least 15% greater, at least 20% greater, at least 25% greater, at least 30% greater, at least 40% greater, at least 50% greater, at least 60% greater, at least 70% greater, at least 80% greater, at least 90%, greater or at least 100% greater. In yet other aspects of this embodiment, the number of hair shafts produce per hair follicle in a treated epidermal region relative to an untreated epidermal region is, e.g., two or more hair shafts/follicle, three or more hair shafts/follicle, four or more hair shafts/follicle, or five or more hair shafts/follicle. In still other aspects of this embodiment, the number of hair shafts produce per hair follicle in a treated epidermal region relative to an untreated epidermal region is, e.g., two hair shafts/follicle, three hair shafts/follicle, four hair shafts/follicle, or five hair shafts/follicle. In further aspects of this embodiment, the number of hair shafts produce per hair follicle in a treated epidermal region relative to an untreated epidermal region is, e.g., two to five hair shafts/follicle, three to five hair shafts/follicle, tow to four hair shafts/follicle, or two to three hair shafts/follicle.

In another embodiment, a method of increasing penetration of a hair follicle into the dermis of an epidermal region from an individual comprises the step of administering a therapeutically effective amount of a composition comprising a prostaglandin, a prostamide, a prostaglandin-glycerol ester, or analog thereof as disclosed in the present specification to the individual, wherein the administration results in an increased penetration of a hair follicle into the dermis. In aspects of this embodiment, the increased penetration of a hair follicle into the dermis from a treated epidermal region relative to an untreated epidermal region is, e.g., about 5% greater, about 10% greater, about 15% greater, about 20% greater, about 25% greater, about 30% greater, about 40% greater, about 50% greater, about 60% greater, about 70% greater, about 80% greater, about 90% greater, or about 100% greater. In other aspects of this embodiment, the increased penetration of a hair follicle into the dermis from a treated epidermal region relative to an untreated epidermal region is, e.g., at least 5% greater, at least 10% greater, at least 15% greater, at least 20% greater, at least 25% greater, at least 30% greater, at least 40% greater, at least 50% greater, at least 60% greater, at least 70% greater, at least 80% greater, at least 90%, greater or at least 100% greater. In yet other aspects of this embodiment, the increased penetration of a hair follicle into the dermis from a treated epidermal region relative to an untreated epidermal region is, e.g., at least 100 μm, at least 200 μm, at least 300 μm, at least 400 μm, at least 500 μm, at least 600 μm, at least 700 μm, at least 800 μm, at least 900 μm, at least 1 mm, at least 2 mm, at least 3 mm, at least 4 mm, or at least 5 mm. In still other aspects of this embodiment, the increased penetration of a hair follicle into the dermis from a treated epidermal region relative to an untreated epidermal region is, e.g., about 100 μm, about 200 μm, about 300 μm, about 400 μm, about 500 μm, about 600 μm, about 700 μm, about 800 μm, about 900 μm, about 1 mm, about 2 mm, about 3 mm, about 4 mm, or about 5 mm. In further aspects of this embodiment, the increased penetration of a hair follicle into the dermis from a treated epidermal region relative to an untreated epidermal region is, e.g., about 100 μm to about 5 mm, about 100 μm to about 4 mm, about 100 μm to about 3 mm, about 100 μm to about 2 mm, about 100 μm to about 1 mm, or about 100 μm to about 500 μm.

In another embodiment, a method of increasing pigmentation of a hair shaft in an epidermal region of an individual comprises the step of administering a therapeutically effective amount of a composition comprising a prostaglandin, a prostamide, a prostaglandin-glycerol ester, or analog thereof as disclosed in the present specification to the individual, wherein the administration results in an increased pigmentation of the hair shaft. In aspects of this embodiment, the increased hair shaft pigmentation from a treated epidermal region relative to an untreated epidermal region is, e.g., about 5% greater, about 10% greater, about 15% greater, about 20% greater, about 25% greater, about 30% greater, about 40% greater, about 50% greater, about 60% greater, about 70% greater, about 80% greater, about 90% greater, or about 100% greater. In other aspects of this embodiment, the increased hair shaft pigmentation from a treated epidermal region relative to an untreated epidermal region is, e.g., at least 5% greater, at least 10% greater, at least 15% greater, at least 20% greater, at least 25% greater, at least 30% greater, at least 40% greater, at least 50% greater, at least 60% greater, at least 70% greater, at least 80% greater, at least 90%, greater or at least 100% greater.

In another embodiment, a method of increasing melanization of a hair shaft from an epidermal region of an individual comprises the step of administering a therapeutically effective amount of a composition comprising a prostaglandin, a prostamide, a prostaglandin-glycerol ester, or analog thereof as disclosed in the present specification to the individual, wherein the administration results in an increased melanization of the hair shaft. In aspects of this embodiment, the increased hair shaft melanization from a treated epidermal region relative to an untreated epidermal region is, e.g., about 5% greater, about 10% greater, about 15% greater, about 20% greater, about 25% greater, about 30% greater, about 40% greater, about 50% greater, about 60% greater, about 70% greater, about 80% greater, about 90% greater, or about 100% greater. In other aspects of this embodiment, the increased hair shaft melanization from a treated epidermal region relative to an untreated epidermal region is, e.g., at least 5% greater, at least 10% greater, at least 15% greater, at least 20% greater, at least 25% greater, at least 30% greater, at least 40% greater, at least 50% greater, at least 60% greater, at least 70% greater, at least 80% greater, at least 90%, greater or at least 100% greater.

In another embodiment, a method of increasing hair shaft luster in an epidermal region of an individual comprises the step of administering a therapeutically effective amount of a composition comprising a prostaglandin, a prostamide, a prostaglandin-glycerol ester, or analog thereof as disclosed in the present specification to the individual, wherein the administration results in an increased hair shaft luster. In aspects of this embodiment, the increased hair shaft luster from a treated epidermal region relative to an untreated epidermal region is, e.g., about 5% greater, about 10% greater, about 15% greater, about 20% greater, about 25% greater, about 30% greater, about 40% greater, about 50% greater, about 60% greater, about 70% greater, about 80% greater, about 90% greater, or about 100% greater. In other aspects of this embodiment, the increased hair shaft luster from a treated epidermal region relative to an untreated epidermal region is, e.g., at least 5% greater, at least 10% greater, at least 15% greater, at least 20% greater, at least 25% greater, at least 30% greater, at least 40% greater, at least 50% greater, at least 60% greater, at least 70% greater, at least 80% greater, at least 90%, greater or at least 100% greater.

In another embodiment, a method of increasing hair health in an epidermal region of an individual comprises the step of administering a therapeutically effective amount of a composition comprising a prostaglandin, a prostamide, a prostaglandin-glycerol ester, or analog thereof as disclosed in the present specification to the individual, wherein the administration results in an increased hair health. In aspects of this embodiment, the increased hair health from a treated epidermal region relative to an untreated epidermal region is, e.g., about 5% greater, about 10% greater, about 15% greater, about 20% greater, about 25% greater, about 30% greater, about 40% greater, about 50% greater, about 60% greater, about 70% greater, about 80% greater, about 90% greater, or about 100% greater. In other aspects of this embodiment, the increased hair health from a treated epidermal region relative to an untreated epidermal region is, e.g., at least 5% greater, at least 10% greater, at least 15% greater, at least 20% greater, at least 25% greater, at least 30% greater, at least 40% greater, at least 50% greater, at least 60% greater, at least 70% greater, at least 80% greater, at least 90%, greater or at least 100% greater.

In another embodiment, a method of increasing keratinization of a hair shaft in an epidermal region of an individual comprises the step of administering a therapeutically effective amount of a composition comprising a prostaglandin, a prostamide, a prostaglandin-glycerol ester, or analog thereof as disclosed in the present specification to the individual, wherein the administration results in an increased keratinization of the hair shaft. In aspects of this embodiment, the increased hair shaft keratinization from a treated epidermal region relative to an untreated epidermal region is, e.g., about 5% greater, about 10% greater, about 15% greater, about 20% greater, about 25% greater, about 30% greater, about 40% greater, about 50% greater, about 60% greater, about 70% greater, about 80% greater, about 90% greater, or about 100% greater. In other aspects of this embodiment, the increased hair shaft keratinization from a treated epidermal region relative to an untreated epidermal region is, e.g., at least 5% greater, at least 10% greater, at least 15% greater, at least 20% greater, at least 25% greater, at least 30% greater, at least 40% greater, at least 50% greater, at least 60% greater, at least 70% greater, at least 80% greater, at least 90%, greater or at least 100% greater. In yet another aspect of this embodiment, increased keratinization of the hair shaft results in increase keratin deposition within hair shafts from a treated epidermal region relative to an untreated epidermal region that is, e.g., about 5% greater, about 10% greater, about 15% greater, about 20% greater, about 25% greater, about 30% greater, about 40% greater, about 50% greater, about 60% greater, about 70% greater, about 80% greater, about 90% greater, or about 100% greater. In still another aspect of this embodiment, increased keratinization of the hair shaft results in increase keratin deposition within hair shafts from a treated epidermal region relative to an untreated epidermal region that is, e.g., at least 5% greater, at least 10% greater, at least 15% greater, at least 20% greater, at least 25% greater, at least 30% greater, at least 40% greater, at least 50% greater, at least 60% greater, at least 70% greater, at least 80% greater, at least 90%, greater or at least 100% greater.

In another embodiment, a method of increasing the time a hair follicle remains in anagen phase in an epidermal region of an individual comprises the step of administering a therapeutically effective amount of a composition comprising a prostaglandin, a prostamide, a prostaglandin-glycerol ester, or analog thereof as disclosed in the present specification to the individual, wherein the administration results in an increased time the hair follicle remains in anagen phase. In aspects of this embodiment, the increased time hair follicles remains in anagen phase from a treated epidermal region relative to an untreated epidermal region is, e.g., about 5% greater, about 10% greater, about 15% greater, about 20% greater, about 25% greater, about 30% greater, about 40% greater, about 50% greater, about 60% greater, about 70% greater, about 80% greater, about 90% greater, or about 100% greater. In other aspects of this embodiment, the increased time hair follicles remains in anagen phase from a treated epidermal region relative to an untreated epidermal region is, e.g., at least 5% greater, at least 10% greater, at least 15% greater, at least 20% greater, at least 25% greater, at least 30% greater, at least 40% greater, at least 50% greater, at least 60% greater, at least 70% greater, at least 80% greater, at least 90%, greater or at least 100% greater. In yet other aspects of this embodiment, the increased time hair follicles remains in anagen phase from a treated epidermal region relative to an untreated epidermal region is, e.g., at least 1 week more, at least 2 weeks more, at least 3 weeks more, at least 4 weeks more, at least 5 weeks more, at least 6 weeks more, at least 7 weeks more, at least 8 weeks more, at least 9 weeks more, at least 10 weeks more, at least 11 weeks more, or at least 12 weeks more. In still other aspects of this embodiment, the increased time hair follicles remains in anagen phase from a treated epidermal region relative to an untreated epidermal region is, e.g., at least 1 month more, at least 2 months more, at least 3 months more, at least 4 months more, at least 5 months more, at least 6 months more, at least 7 months more, at least 8 months more, at least 9 months more, at least 10 months more, at least 11 months more, or at least 12 months more. In further aspects of this embodiment, the increased time hair follicles remains in anagen phase from a treated epidermal region relative to an untreated epidermal region is, e.g., about 1 week to 12 weeks more, about 1 month to 12 months more, about 1 year to about 5 years more.

In another embodiment, a method of increasing the time a hair follicle remains in catagen phase in an epidermal region of an individual comprises the step of administering a therapeutically effective amount of a composition comprising a prostaglandin, a prostamide, a prostaglandin-glycerol ester, or analog thereof as disclosed in the present specification to the individual, wherein the administration results in an increased time the hair follicle remains in catagen phase. In aspects of this embodiment, the increased time hair follicles remains in catagen phase from a treated epidermal region relative to an untreated epidermal region is, e.g., about 5% greater, about 10% greater, about 15% greater, about 20% greater, about 25% greater, about 30% greater, about 40% greater, about 50% greater, about 60% greater, about 70% greater, about 80% greater, about 90% greater, or about 100% greater. In other aspects of this embodiment, the increased time hair follicles remains in catagen phase from a treated epidermal region relative to an untreated epidermal region is, e.g., at least 5% greater, at least 10% greater, at least 15% greater, at least 20% greater, at least 25% greater, at least 30% greater, at least 40% greater, at least 50% greater, at least 60% greater, at least 70% greater, at least 80% greater, at least 90%, greater or at least 100% greater. In yet other aspects of this embodiment, the increased time hair follicles remains in catagen phase from a treated epidermal region relative to an untreated epidermal region is, e.g., at least 1 week more, at least 2 weeks more, at least 3 weeks more, at least 4 weeks more, at least 5 weeks more, at least 6 weeks more, at least 7 weeks more, at least 8 weeks more, at least 9 weeks more, at least 10 weeks more, at least 11 weeks more, or at least 12 weeks more. In still other aspects of this embodiment, the increased time hair follicles remains in catagen phase from a treated epidermal region relative to an untreated epidermal region is, e.g., at least 1 month more, at least 2 months more, at least 3 months more, at least 4 months more, at least 5 months more, at least 6 months more, at least 7 months more, at least 8 months more, at least 9 months more, at least 10 months more, at least 11 months more, or at least 12 months more. In further aspects of this embodiment, the increased time hair follicles remains in catagen phase from a treated epidermal region relative to an untreated epidermal region is, e.g., about 1 week to 12 weeks more, about 1 month to 12 months more, about 1 year to about 5 years more.

In another embodiment, a method of increasing the time a hair follicle remains in telogen phase in an epidermal region of an individual comprises the step of administering a therapeutically effective amount of a composition comprising a prostaglandin, a prostamide, a prostaglandin-glycerol ester, or analog thereof as disclosed in the present specification to the individual, wherein the administration results in an increased time the hair follicle remains in telogen phase. In aspects of this embodiment, the increased time hair follicles remains in telogen phase from a treated epidermal region relative to an untreated epidermal region is, e.g., about 5% greater, about 10% greater, about 15% greater, about 20% greater, about 25% greater, about 30% greater, about 40% greater, about 50% greater, about 60% greater, about 70% greater, about 80% greater, about 90% greater, or about 100% greater. In other aspects of this embodiment, the increased time hair follicles remains in telogen phase from a treated epidermal region relative to an untreated epidermal region is, e.g., at least 5% greater, at least 10% greater, at least 15% greater, at least 20% greater, at least 25% greater, at least 30% greater, at least 40% greater, at least 50% greater, at least 60% greater, at least 70% greater, at least 80% greater, at least 90%, greater or at least 100% greater. In yet other aspects of this embodiment, the increased time hair follicles remains in telogen phase from a treated epidermal region relative to an untreated epidermal region is, e.g., at least 1 week more, at least 2 weeks more, at least 3 weeks more, at least 4 weeks more, at least 5 weeks more, at least 6 weeks more, at least 7 weeks more, at least 8 weeks more, at least 9 weeks more, at least 10 weeks more, at least 11 weeks more, or at least 12 weeks more. In still other aspects of this embodiment, the increased time hair follicles remains in telogen phase from a treated epidermal region relative to an untreated epidermal region is, e.g., at least 1 month more, at least 2 months more, at least 3 months more, at least 4 months more, at least 5 months more, at least 6 months more, at least 7 months more, at least 8 months more, at least 9 months more, at least 10 months more, at least 11 months more, or at least 12 months more. In further aspects of this embodiment, the increased time hair follicles remains in telogen phase from a treated epidermal region relative to an untreated epidermal region is, e.g., about 1 week to 12 weeks more, about 1 month to 12 months more, about 1 year to about 5 years more.

In another embodiment, a method of prolonging or preventing the release of a hair shaft from a hair follicle in an epidermal region of an individual comprises the step of administering a therapeutically effective amount of a composition comprising a prostaglandin, a prostamide, a prostaglandin-glycerol ester, or analog thereof as disclosed in the present specification to the individual, wherein the administration prolongs or prevents the release of the hair shaft from the hair follicle. In aspects of this embodiment, the length of time before a hair shaft is released from a follicle in a treated epidermal region relative to an untreated epidermal region is, e.g., about 5% greater, about 10% greater, about 15% greater, about 20% greater, about 25% greater, about 30% greater, about 40% greater, about 50% greater, about 60% greater, about 70% greater, about 80% greater, about 90% greater, or about 100% greater. In other aspects of this embodiment, the length of time before a hair shaft is released from a follicle in a treated epidermal region relative to an untreated epidermal region is, e.g., at least 5% greater, at least 10% greater, at least 15% greater, at least 20% greater, at least 25% greater, at least 30% greater, at least 40% greater, at least 50% greater, at least 60% greater, at least 70% greater, at least 80% greater, at least 90%, greater or at least 100% greater. In yet other aspects of this embodiment, the length of time before a hair shaft is released from a follicle in a treated epidermal region relative to an untreated epidermal region is, e.g., at least 1 week more, at least 2 weeks more, at least 3 weeks more, at least 4 weeks more, at least 5 weeks more, at least 6 weeks more, at least 7 weeks more, at least 8 weeks more, at least 9 weeks more, at least 10 weeks more, at least 11 weeks more, or at least 12 weeks more. In still other aspects of this embodiment, the length of time before a hair shaft is released from a follicle in a treated epidermal region relative to an untreated epidermal region is, e.g., at least 1 month more, at least 2 months more, at least 3 months more, at least 4 months more, at least 5 months more, at least 6 months more, at least 7 months more, at least 8 months more, at least 9 months more, at least 10 months more, at least 11 months more, or at least 12 months more. In further aspects of this embodiment, the length of time before a hair shaft is released from a follicle in a treated epidermal region relative to an untreated epidermal region is, e.g., about 1 week to 12 weeks more, about 1 month to 12 months more, about 1 year to about 5 years more.

In another embodiment, a method of prolonging or preventing the initiation of apoptosis in a hair follicle in an epidermal region of an individual comprises the step of administering a therapeutically effective amount of a composition comprising a prostaglandin, a prostamide, a prostaglandin-glycerol ester, or analog thereof as disclosed in the present specification to the individual, wherein the administration results in a reduction of hair loss, hair thinning, or hair color loss. In aspects of this embodiment, the length of time before a hair follicle initiates apoptosis from a treated epidermal region relative to an untreated epidermal region is, e.g., about 5% greater, about 10% greater, about 15% greater, about 20% greater, about 25% greater, about 30% greater, about 40% greater, about 50% greater, about 60% greater, about 70% greater, about 80% greater, about 90% greater, or about 100% greater. In other aspects of this embodiment, the length of time before a hair follicle initiates apoptosis from a treated epidermal region relative to an untreated epidermal region is, e.g., at least 5% greater, at least 10% greater, at least 15% greater, at least 20% greater, at least 25% greater, at least 30% greater, at least 40% greater, at least 50% greater, at least 60% greater, at least 70% greater, at least 80% greater, at least 90%, greater or at least 100% greater. In yet other aspects of this embodiment, the length of time before a hair follicle initiates apoptosis from a treated epidermal region relative to an untreated epidermal region is, e.g., at least 1 week more, at least 2 weeks more, at least 3 weeks more, at least 4 weeks more, at least 5 weeks more, at least 6 weeks more, at least 7 weeks more, at least 8 weeks more, at least 9 weeks more, at least 10 weeks more, at least 11 weeks more, or at least 12 weeks more. In still other aspects of this embodiment, the length of time before a hair follicle initiates apoptosis from a treated epidermal region relative to an untreated epidermal region is, e.g., at least 1 month more, at least 2 months more, at least 3 months more, at least 4 months more, at least 5 months more, at least 6 months more, at least 7 months more, at least 8 months more, at least 9 months more, at least 10 months more, at least 11 months more, or at least 12 months more. In further aspects of this embodiment, the length of time before a hair follicle initiates apoptosis from a treated epidermal region relative to an untreated epidermal region is, e.g., about 1 week to 12 weeks more, about 1 month to 12 months more, about 1 year to about 5 years more.

The term “hair” refers to any type of hair produced by a mammal, including without limitation, terminal hair, vellus hair, and modified terminal hair present on the skin surface, such as, e.g., hair of the scalp, face, beard, head, pubic area, upper lip, eyebrows, and eyelids. A mammal produces different types of hair, including, without exception, terminal hairs and vellus hairs and modified terminal hairs, such as seen in eye lashes and eyebrows. Terminal hairs are coarse, pigmented, long hairs in which the bulb of the hair follicle is seated deep in the dermis. Terminal hair is developed hair, which is generally longer, coarser, thicker and darker than the shorter and finer vellus hair. Phases of growth in terminal hair are more apparent than in vellus hair; it generally has a longer anagen phase. It has associated sebaceous glands, whereas a vellus hair may not. Under certain conditions, such as puberty, some vellus hair may become terminal hair. Under other conditions, such as male pattern baldness, it may revert to a vellus-like state.

Vellus hairs, on the other hand, are fine, thin, underpigmented or non-pigmented short hairs (commonly referred to as peach fuzz) in which the hair bulb is located superficially in the dermis. It is a very soft, generally pale, and short hair that grows in most places on the human body in both sexes. It is usually less than two centimeters long and the follicles are not connected to sebaceous glands. It is most easily observed in women and children, as they have less terminal hair to obscure it. It is also found in pre-adolescents and in male pattern baldness. Much of human hair is vellus hair rather than terminal hair.

The prostaglandins, prostamides, prostaglandin-glycerol esters, and analogs thereof disclosed in the present specification can treat a condition associated with hair loss, hair thinning, or hair color loss by stimulating the production of new hair follicles. Without wishing to be bound by any particular theory, it is thought that the disclosed prostaglandins, prostamides, prostaglandin-glycerol esters, and analogs thereof can covert vellus hair to terminal hair. Therefore, at any given time during treatment, there are more terminal hairs. The result is an increase in hair length, hair thickness and hair pigmentation.

Thus, in an embodiment, a condition associated with hair thinning in an individual is treated by reducing an attribute associated with hair thinning. In aspects of this embodiment, the attribute associated with hair thinning is reduced by converting vellus hair to terminal hair.

In another embodiment, a method of converting vellus hair to terminal hair in an epidermal region of an individual comprises the step of administering a therapeutically effective amount of a composition comprising a prostaglandin, a prostamide, a prostaglandin-glycerol ester, or analog thereof as disclosed in the present specification to the individual, wherein the administration results in a conversion vellus hair to terminal hair. In aspects of this embodiment, conversion of vellus hair to terminal hair from a treated epidermal region relative to an untreated epidermal region is, e.g., about 5% more, about 10% more, about 15% more, about 20% more, about 25% more, about 30% more, about 40% more, about 50% more, about 60% more, about 70% more, about 80% more, about 90% more, or about 100% more. In other aspects of this embodiment, conversion of vellus hair to terminal hair from a treated epidermal region relative to an untreated epidermal region is, e.g., at least 5% more, at least 10% more, at least 15% more, at least 20% more, at least 25% more, at least 30% more, at least 40% more, at least 50% more, at least 60% more, at least 70% more, at least 80% more, at least 90%, more or at least 100% more.

Aspect of the present specification disclose, in part, administering prostaglandins, prostamides, prostaglandin-glycerol esters, and analogs thereof disclosed in the present specification to an epidermal region of an individual. An epidermal region is any region of the skin that can support hair growth, including, without limitation, a scalp region, an eyebrow region, an eyelash region, a public hair region, a leg region, or an arm region. As used herein, the term “epidermal region” comprises the epidermis, underlying dermis, and any other skin layers necessary to support hair growth.

The term “eyebrow” refers to an area of coarse skin hairs above the eye that follows the shape of the brow ridges. The main function of the eyebrow is to prevent moisture, mostly salty sweat and rain, from flowing into the eye, an organ critical to sight. The typical curved shape of the eyebrow (with a slant on the side) and the direction in which eyebrow hairs are pointed, make sure that moisture has a tendency to flow sideways around the eyes, along the side of the head and along the nose. Eyebrows also prevent debris such as dandruff and other small objects from falling into the eyes, as well as providing a more sensitive sense for detecting objects being near the eye, like small insects. Eyebrows also have an important facilitative function in communication, strengthening facial expressions such as surprise, confusion, or anger.

The term “eyebrow region” as used herein generally describes the region in and around the eyebrow. The eyebrow region can include the entire eyebrow hair, portions thereof, or areas larger than the existing eyebrow hair growth region. For example, eyebrow region can include areas surrounding the existing eyebrow hair if a user would like to grow hair beyond its already existing area.

The terms “eyelash” and “lash” are used interchangeably to refer to one of the hairs that grow at the edge of the eyelid. Eyelashes protect the eye from debris and provide a warning that an object (such as an insect or dust mite) is near the eye (which then is closed reflexively).

The term “eyelash region” as used herein generally describes the region in and around the eyelash. The eyelash region can include the entire eyelash hair, portions thereof, or areas larger than the existing eyelash hair growth region. For example, eyelash region can include areas surrounding the existing eyelash hair if a user would like to grow hair beyond its already existing area.

The term “scalp” refers to the integument of the upper part of the head, usually including the associated subcutaneous structures. The scalp is the anatomical area bordered by the face anteriorly and the neck to the sides and posteriorly.

The term “scalp region” as used herein generally describes the region in and around the scalp. The scalp region can include the entire scalp hair, portions thereof, or areas larger than the existing scalp hair growth region. For example, scalp region can include areas surrounding the existing scalp hair if a user would like to grow hair beyond its already existing area.

The inside of the nose contains small hairs called cilia. These cilia and nasal mucus clean the air drawn into the nose of the microscopic particles we inhale, including dust, pollen, and pollutants, for ultimate passage to the lungs.

Aspects of the present specification disclose, in part, a method for treating a condition associated with hair loss. The hair growth enhancing composition disclosed herein was discovered during unilateral treatment of patients with glaucoma. It was noted that the treated eye had noticeably longer, thicker, fuller eyelashes compared to the lashes on the untreated eye. These findings, which are disclosed in U.S. Pat. No. 7,351,404 and are incorporated herein by reference, were unexpected and surprising and led to the development of methods for enhancing eyebrow hair growth as described herein.

The term “hair loss” refers to the absence or loss of hair from a skin surface, including, without limitation, hair loss from the scalp, face, beard, head, pubic area, upper lip, eyebrows, and/or eyelids. Alopecia is a medical term for hair loss. Alopecia can be caused by a multitude of factors including, without limitation, genetic make-up, functional disorder, hereditary disorder, hereditary disposition of the hair shaft or genodermatoses, chemical breakage such as over processing, or frequent use of chemical relaxer, heat damage as from repeated hot comb use, chronic exposure to traction on hair shaft, compulsive hair pulling, telogen effluvium resulting from physical or psychological stress, secondary syphilis can cause “moth eaten hairloss”, discoid lupus erythematosus or chronic cutaneous lupus erythematosus, lichenplanopilaris, pseudopelade of Brocq, tufted folliculitis, dissecting cellulitis, alopecia mucinosa, keratosis follicularis spinulosa decalvans, adverse effect from certain drugs such as chemotherapy, radiation therapy, and testosterone booster tablets. Alopecia frequently occurs in patients undergoing treatment for cancer or suffering from other diseases, such as AIDS, where cell-killing, or cytotoxic, drugs are used.

Alopecia is typically categorized as scarring or nonscarring. Scarring alopecia, also known as “alopecia cicatrisata” or “cicatricial alopecia,” refers to hair loss characterized by potentially permanent and irreversible destruction of hair follicles and their replacement with scar tissue. Non-limiting examples include bullous diseases, chemical alopecia, discoid lupus erythematosus, severre folliculitis, lichen planopilaris, dissecting cellulitis, central centrifugal cicatricial alopecia, postmenopausal frontal fibrosing alopecia, and tumors and skin outgrowths, such as, e.g., sebaceous nevus, basal cell carcinoma, and squamous cell carcinoma.

Nonscarring alopecia refers to hair loss without permanent destruction of the hair follicle. Non-limiting examples include anagen effluvium, alopecia adnata, alopecia androgenetica, alopecia areata, alopecia congenitalis, alopecia diffusa, alopecia disseminate, alopecia follicularis, alopecia leprotica, alopecia marginalis, alopecia medicamentosa, alopecia mucinosa, alopecia neurotica, alopecia pityrodes, alopecia presenili, alopecia senilis, alopecia symptomatica, alopecia syphilitica, alopecia totalis, alopecia toxica, alopecia triangularis, alopecia triangularis congenitalis, alopecia universalis, folliculitis, olliculitis decalvans, traction alopecia, trichotillomania, telogen effluvium, and inherited disorders of the hair shaft.

The term “anagen effluvium” refers to the hair loss associated with chemotherapeutic agents used in chemotherapy or radiotherapy that cause immediate destruction and release of anagen hair.

The term “alopecia adnata” refers to a condition involving the loss of eyelash hair.

The term “alopecia androgenetica” also called “androgenetic alopecia” or “androgenic alopecia” refers to a gradual decrease of scalp hair density in adults with transformation of terminal to vellus hairs, which become lost as a result of familial increased susceptibility of hair follicles to androgen secretion following puberty. The most common form of androgenic alopecia is male pattern baldness. Male-pattern baldness is the most common cause of hair loss in men. Men who have this type of hair loss usually have inherited the trait. Men who start losing their hair at an early age tend to develop more extensive baldness. In male-pattern baldness, hair loss typically results in a receding hair line and baldness on the top of the head. The most common form of androgenic alopecia in women is female pattern alopecia, a diffuse partial hair loss in the centroparietal area of the scalp, with preservation of the frontal and temporal hairlines. When it occurs in females, it is associated with other evidence of excessive androgen activity, such as hirsutism.

With the onset of alopecia androgenetica, a successively greater proportion of the hairs are in the telogen phase with correspondingly fewer in the active growth anagen phase. For instance, a bald human subject will average an anagen: telogen ration of about 70:30, whereas, a non-bald human in the same age group will have an average an anagen: telogen ration of about 90:10. In addition, a transition takes place in the area of approaching baldness wherein the hairs themselves are changing from the terminal to the vellus type. Furthermore, androgenic alopecia is associated with the severe diminution of hair follicle numbers. For instance, a bald human subject will average only about 306 follicles per square centimeter, whereas, a non-bald human in the same age group will have an average of 460 follicles per square centimeter. This amounts to a one-third reduction in hair follicles which, when added to the increased proportion of vellus hair follicles and the increased number of hair follicles in the telogen phase, is both significant and noticeable. Approximately 50% of the hairs must be shed to produce visible thinning of scalp hair due to hair loss. It is thus a combination of these factors: transition of hairs from terminal to vellus, increased number of telogen hairs—some of which have been shed, and loss of hair follicles that produces “baldness.”

The term “alopecia areata” or “spot baldness” refers to a condition involving the loss of hair in a patchy pattern. Hair loss usually occurs in asymmetrical areas on the scalp, eyebrows, and beaded portion of the face. Alopecia areata is thought to be an autoimmune disorder occurring on areas of the body (most commonly the scalp) where the person's immune system attacks hair follicles, thereby suppressing and arresting hair growth. Alopecia areata can result in hair loss ranging from just one location (alopecia areata monolocularis) to every hair on the entire body (alopecia areata universalis).

The term “alopecia congenitalis” refers to a congenital condition involving the loss of all hair at birth. alopecia congenitalis is usually associated with psychomotor epilepsy.

The term “alopecia diffusa” or “diffuse alopecia” refers to a condition involving a gradual loss of hair across the whole scalp. It occurs primarily in females for a variety of reasons.

The term “alopecia disseminata” refers to a condition involving the loss of hair from all parts of the body.

The term “alopecia follicularis” refers to a congenital condition involving the loss of hair due to inflammation of hair follicles.

The term “alopecia leprotica” refers to a condition involving the partial or total loss of hair from the lateral third of the eyebrows, eyelashes, and body hairs, and rarely scalp. This condition is seen in leprosy.

The term “alopecia marginalis” refers to a condition involving the loss of hair at the hair line of the scalp.

The term “alopecia medicamentosa” refers to a condition involving the diffuse loss of hair, most notably of the scalp. Usually caused by administration of various types of drugs.

The term “alopecia mucinosa” refers to a condition involving the loss of hair in areas of erythema and oedema in the bearded portion of the face or in the scalp, usually associated with follicular mucinosis.

The term “alopecia neurotica” refers to a congenital condition involving the loss of hair due to a nervous disorder or injury to the nervous system.

The term “alopecia pityrodes” refers to a condition involving the loss of hair of the body as well as of the scalp, accompanied by an abundant branlike desquamation.

The term “alopecia presenilis” refers to a condition involving the loss of hair during early or middle life without any apparent disease of the scalp.

The term “alopecia senilis” refers to a condition involving the loss of hair from scalp due to old age.

The term “alopecia symptomatica” refers to a condition involving the loss of hair occurring in the course of various constitutional or local diseases, or following prolonged febrile illness.

The term “alopecia syphilitica” refers to a condition involving the loss of hair due to syphilis.

The term “alopecia totalis” refers to a condition involving the loss of all head hair.

The term “alopecia toxica” refers to a condition involving the loss of hair due to febrile illness.

The term “alopecia triangularis” refers to a condition involving the loss of hair due to bilateral receding temporal hair lines of the scalp.

The term “alopecia triangularis congenitalis” refers to a congenital condition involving the loss of hair as a triangular patch on the frontal or temporal region of the scalp.

The term “alopecia universalis” refers to a condition involving the loss of all hair from the head and the body.

The term “folliculitis” refers to a condition involving the loss of hair due to inflammation of the hair follicle, usually by a bacteria or fungus infection. Folliculitis can also be caused when hair follicles are damaged by friction from clothing, an insect bite, blockage of the follicle, shaving, or tight braids too close to the scalp.

The term “folliculitis decalvans” or “acne decalvans,” “alopecia follicularis”, or “tufted folliculitis” refers to a condition involving the loss of hair from the scalp due to inflammation of the hair follicle.

The term “traction alopecia”, “traumatic alopecia”, or “chemical alopecia” refers to a condition involving the loss of hair due to chronic or repetitive exposure to traction on the hair by pulling or twisting or by exposure to a chemical, caustic agent, or heat. Hair loss can be circumscribed or diffuse, temporary or permanent. Alopecia marginalis is a form of traction alopecia.

The term “trichotillomania” refers to a condition involving the loss of hair due to a psychological compulsion to pull out or bend one's own hair. It tends to occur more in children than in adults. In this condition the hairs are not absent from the scalp but are broken.

The term “telogen effluvium” refers to a condition involving the loss of hair due to an increased transient shedding of normal club hairs by premature progression to the telogen phase in anagen follicles. Usually there is an abrupt shift of large numbers of anagen hairs to telogen hairs on the scalp, with a corresponding change in the ratio of anagen hair to telogen hair from the normal ratio of 90:10 to 70:30. This form of alopecia generally begins approximately 3 months after a physical or psychological stress or trauma, such as, e.g., illness, disease like diabetes, syphilis, hypothyroidism, or hyperthyroidism, surgery, medical treatments like chemotherapy or radiotherapy, medications like anticoagulants (blood thinners), gout medication, high blood pressure, or heart problems, excessive vitamin A, birth control pills, fertility-stimulating drugs like clomiphene, and antidepressants, poisoning, parturition (childbirth), hormonal exposure or derangement like androgen and/or estrogen imbalance, rapid weight loss, nutritional deficiency like a mineral or vitamin deficiency (iron deficiency), infections like fungal (mycotic) infections such as “black dot”, ringworm, tinea, or tinea capitis, high fever, or hemorrhage).

Thus, in an embodiment, a composition comprising a prostaglandin, prostamide, prostaglandin-glycerol ester, or analog thereof disclosed in the present specification is administered to an individual to treat hair loss. In an aspect of this embodiment, a composition disclosed in the present specification is administered to an individual to treat hair loss associated with scarring alopecia. In other aspects of this embodiment, a composition disclosed in the present specification is administered to an individual to treat scarring alopecia associated with a bullous disease, a chemical exposure, a discoid lupus erythematosus, a severre folliculitis, a lichen planopilaris, a dissecting cellulitis, a central centrifugal cicatricial alopecia, a postmenopausal frontal fibrosing alopecia, a tumor, or a skin outgrowth.

In another aspect of this embodiment, a composition disclosed in the present specification is administered to an individual to treat a hair loss associated with non-scarring alopecia. In other aspects of this embodiment, a composition disclosed in the present specification is administered to an individual to treat hair loss associated anagen effluvium, alopecia adnata, alopecia androgenetica, alopecia areata, alopecia congenitalis, alopecia diffusa, alopecia disseminate, alopecia follicularis, alopecia leprotica, alopecia marginalis, alopecia medicamentosa, alopecia mucinosa, alopecia neurotica, alopecia pityrodes, alopecia presenili, alopecia senilis, alopecia symptomatica, alopecia syphilitica, alopecia totalis, alopecia toxica, alopecia triangularis, alopecia triangularis congenitalis, alopecia universalis, folliculitis, olliculitis decalvans, traction alopecia, trichotillomania, telogen effluvium, or inherited disorder of the hair shaft.

In an aspect of this embodiment, a composition disclosed in the present specification is administered to an individual to treat a hair loss associated with no new hair shaft growth, reduced rate of hair shaft growth, reduced hair shaft diameter (thickness), reduced hair shaft length, reduced hair density, reduced hair pigmentation, reduced hair shaft luster, reduced hair health, reduced time a hair follicle spends in anagen phase, reduced time a hair follicle spends in catagen phase, reduced time a hair follicle spends in telogen phase, premature release of hair shaft from hair follicle, premature initiation of apoptosis in hair follicle, premature conversion of a terminal hair into a vellus hair.

In another aspect of this embodiment, hair loss in an individual is treated by reducing an attribute associated with hair loss. In aspects of this embodiment, reduction of the attribute associated with hair loss is by increasing the rate of hair growth, increasing hair thickness, increasing hair length, increasing hair density, increasing number of hairs produce per follicle, increasing hair pigmentation, increasing hair luster, converting intermediate or vellus hair to terminal hair, increasing hair health, increasing the time a hair follicle remains in anagen phase, increasing the time a hair follicle remains in catagen phase, increasing the time a hair follicle remains in telogen phase, prolonging or preventing the release of the hair shaft from the hair follicle, or prolonging or preventing the initiation of apoptosis in a hair follicle.

Aspects of the present specification disclose, in part, a method for treating a condition associated with hair thinning. The term “hair thinning” refers to an age-related condition where hair follicles produce hairs that are shorter in length, smaller in diameter, lighter in color, and more fragile as opposed to no hair production at all. Hair thinning is a condition where the shaft of each hair becomes shorter in length, smaller in diameter (finer), less pigmented, and/or more fragile. As such, hair thinning is distinct from hair loss, a condition in which the hair follicle stops producing a hair shaft altogether. As discussed above, the hair follicle is a complex mini organ. But like all biological systems, the biologically active part of the hair follicle undergoes an aging process. This aging process is characterized by 1) the migration of the base of the hair follicle upwards toward the skin surface, a decline in the synthesis of hair keratins, and a loss of pigmentation. Although producing hairs, older hair follicles make hairs that are shorter in length, smaller in diameter, lighter in color, and more fragile. Taken together, this age-related shift in hair characteristics manifests itself as hair thinning. Thinning hair affects an estimated 40 million men and 25 million women in the United States. The emotional impact from hair loss can lead to anxiety, stress, depression, and lower self esteem.

Thus, in an embodiment, a composition comprising a prostaglandin, prostamide, prostaglandin-glycerol ester, or analog thereof disclosed in the present specification is administered to an individual to treat hair thinning. In another aspect of this embodiment, a composition comprising a prostaglandin, prostamide, prostaglandin-glycerol ester, or analog thereof disclosed in the present specification is administered to an individual to treat an attribute associated with hair thinning, wherein the administration results in a reduction in the attribute associated with hair thinning. In an aspect of this embodiment, a composition disclosed in the present specification is administered to an individual to treat hair thinning associated with decreased hair length, decreased hair diameter, decreased keratinization of the hair shaft, increased fragility, reduced hair health, reduced time a hair follicle spends in anagen phase, reduced time a hair follicle spends in catagen phase, reduced time a hair follicle spends in telogen phase, or premature conversion of a terminal hair into a vellus hair.

In aspects of this embodiment, reduction of the attribute associated with hair thinning is by increasing the rate of hair growth, increasing hair thickness, increasing hair length, increasing hair density, increasing number of hairs produce per follicle, increasing keratin production in the hair shaft, increasing hair shaft pigmentation, increasing hair shaft luster, converting intermediate or vellus hair to terminal hair, increasing hair health, increasing the time a hair follicle remains in anagen phase, increasing the time a hair follicle remains in catagen phase, or increasing the time a hair follicle remains in telogen phase.

Aspects of the present specification disclose, in part, a method for treating a condition associated with hair color loss. The term “hair color loss” refers to the reduction of pigmentation of the hair shaft.

Thus, in an embodiment, a composition comprising a prostaglandin, prostamide, prostaglandin-glycerol ester, or analog thereof disclosed in the present specification is administered to an individual to treat hair color loss. In another aspect of this embodiment, a composition comprising a prostaglandin, prostamide, prostaglandin-glycerol ester, or analog thereof disclosed in the present specification is administered to an individual to treat an attribute associated with hair color loss, wherein the administration results in a reduction in the attribute associated with hair color loss. In an aspect of this embodiment, a composition disclosed in the present specification is administered to an individual to treat a hair color loss associated with decreases pigmentation of the hair shaft, decreased melanin production, increased death of melanocytes associated with apoptosis or any other cause.

In aspects of this embodiment, reduction of the attribute associated with hair color loss is by increasing pigmentation of the hair shaft, increasing melanin production, increasing hair luster, converting intermediate or vellus hair to terminal hair, increasing hair health, increasing the time a hair follicle remains in anagen phase, increasing the time a hair follicle remains in catagen phase, increasing the time a hair follicle remains in telogen phase, prolonging or preventing melanocyte death, or prolonging or preventing the initiation of apoptosis in a hair follicle.

Aspects of the present invention provide, in part, a mammal. A mammal includes a human, and a human can be a patient. Other aspects of the present invention provide, in part, an individual. An individual includes a human, and a human can be a patient. A non-human mammal includes any and all animals that are raised or utilized for their fur and pelts such as, e.g., a mink, a chinchilla, fox, or a beaver.

Aspects of the present invention provide, in part, administering a composition comprising a prostaglandin, prostamide, prostaglandin-glycerol ester, or analog thereof disclosed in the present specification. As used herein, the term “administering” means any delivery mechanism that provides a composition comprising a prostaglandin, prostamide, prostaglandin-glycerol ester, or analog thereof disclosed in the present specification to an individual that potentially results in a clinically, therapeutically, or experimentally beneficial result.

A composition comprising a prostaglandin, prostamide, prostaglandin-glycerol ester, or analog thereof disclosed in the present specification can be administered to a mammal using a cellular uptake approach. Administration of a composition comprising a prostaglandin, prostamide, prostaglandin-glycerol ester, or analog thereof disclosed in the present specification using a cellular uptake approach comprise a variety of enteral or parenteral approaches including, without limitation, oral administration in any acceptable form, such as, e.g., tablet, liquid, capsule, powder, or the like; topical administration in any acceptable form, such as, e.g., drops, spray, creams, gels or ointments; intravascular administration in any acceptable form, such as, e.g., intravenous bolus injection, intravenous infusion, intra-arterial bolus injection, intra-arterial infusion and catheter instillation into the vasculature; peri- and intra-tissue administration in any acceptable form, such as, e.g., intraperitoneal injection, intramuscular injection, dermal injection, epidermal injection, subcutaneous injection, subcutaneous infusion, intraocular injection, retinal injection, or sub-retinal injection or epidural injection; intravesicular administration in any acceptable form, such as, e.g., catheter instillation; and by placement device, such as, e.g., an implant, a patch, a pellet, a catheter, an osmotic pump, a suppository, a bioerodible delivery system, a non-bioerodible delivery system or another implanted extended or slow release system. An exemplary list of biodegradable polymers and methods of use are described in, e.g., Handbook of Biodegradable Polymers (Abraham J. Domb et al., eds., Overseas Publishers Association, 1997).

A composition comprising a prostaglandin, prostamide, prostaglandin-glycerol ester, or analog thereof disclosed in the present specification can be administered to an individual by a variety of methods known to those of skill in the art, including, but not restricted to, encapsulation in liposomes, by ionophoresis, or by incorporation into other vehicles, such as hydrogels, cyclodextrins, biodegradable nanocapsules, and bioadhesive microspheres, or by proteinaceous vectors. Delivery mechanisms for administering a composition comprising a prostaglandin, prostamide, prostaglandin-glycerol ester, or analog thereof disclosed in the present specification to an individual are described in, e.g., Leonid Beigelman et al., Compositions for the Delivery of Negatively Charged Molecules, U.S. Pat. No. 6,395,713 (May 28, 2002); and Achim Aigner, Delivery Systems for the Direct Application of siRNAs to Induce RNA Interference (RNAi) in vivo, 2006 (716559) J. Biomed. Biotech. 1-15 (2006); Controlled Drug Delivery: Designing Technologies for the Future (Kinam Park & Randy J. Mrsny eds., American Chemical Association, 2000); Vernon G. Wong & Mae W. L. Hu, Methods for Treating Inflammation-mediated Conditions of the Eye, U.S. Pat. No. 6,726,918 (Apr. 27, 2004); David A. Weber et al., Methods and Apparatus for Delivery of Ocular Implants, U.S. Patent Publication No. US2004/0054374 (Mar. 18, 2004); Thierry Nivaggioli et al., Biodegradable Ocular Implant, U.S. Patent Publication No. US2004/0137059 (Jul. 15, 2004); Patrick M. Hughes et al., Anti-Angiogenic Sustained Release Intraocular Implants and Related Methods, U.S. patent application Ser. No. 11/364,687 (Feb. 27, 2006); and Patrick M. Hughes et al., Sustained Release Intraocular Drug Delivery Systems, U.S. Patent Publication 2006/0182783 (Aug. 17, 2006), each of which is hereby incorporated by reference in its entirety.

A composition comprising a prostaglandin, prostamide, prostaglandin-glycerol ester, or analog thereof disclosed in the present specification can be administered to a mammal using a variety of routes. Routes of administration suitable for a method of treating a condition as disclosed in the present specification include both local and systemic administration. Local administration results in significantly more delivery of a composition to a specific location as compared to the entire body of the individual, whereas, systemic administration results in delivery of a composition to essentially the entire body of the individual. Routes of administration suitable for a method of treating a condition as disclosed in the present specification also include both central and peripheral administration. Central administration results in delivery of a composition to essentially the central nervous system of the patient and includes, e.g., intrathecal administration, epidural administration as well as a cranial injection or implant. Peripheral administration results in delivery of a composition to essentially any area of a patient outside of the central nervous system and encompasses any route of administration other than direct administration to the spine or brain. The actual route of administration of a composition comprising a prostaglandin, prostamide, prostaglandin-glycerol ester, or analog thereof disclosed in the present specification used in an individual can be determined by a person of ordinary skill in the art by taking into account factors, including, without limitation, the type of condition, the location of the condition, the cause of the condition, the severity of the condition, the degree of relief desired, the duration of relief desired, the particular prostaglandin, prostamide, prostaglandin-glycerol ester, or analog thereof used, the rate of excretion of the prostaglandin, prostamide, prostaglandin-glycerol ester, or analog thereof used, the pharmacodynamics of the prostaglandin, prostamide, prostaglandin-glycerol ester, or analog thereof used, the nature of the other compounds to be included in the composition, the particular route of administration, the particular characteristics, history and risk factors of the individual, such as, e.g., age, weight, general health and the like, or any combination thereof.

The compositions contemplated herein include compositions suited for topical and local action for pharmaceutical and cosmetic applications. The term “topical” as employed herein relates to the use of a prostaglandin, prostamide, prostaglandin-glycerol ester, or analog thereof, as described herein, incorporated in a suitable pharmaceutical carrier, and applied at the site of hair loss, hair thinning, and/or hair color loss to the affected epidermal region. Accordingly, such topical compositions include those pharmaceutical forms in which a prostaglandin prostaglandin, prostamide, prostaglandin-glycerol ester, or analog thereof is applied externally by direct contact with the skin surface to be treated. Conventional pharmaceutical forms for this purpose include ointments, liniments, creams, shampoos, lotions, pastes, jellies, sprays, aerosols, and the like, and may be applied in patches or impregnated dressings depending on the part of the epidermal region to the treated. The term “ointment” embraces formulations (including creams) having oleaginous, water-soluble and emulsion-type bases, e.g., petrolatum, lanolin, polyethylene glycols, as well as mixtures of these.

For topical use on an epidermal region, a prostaglandin, prostamide, prostaglandin-glycerol ester, or analog thereof can be advantageously formulated using aqueous solutions, ointments, oils, creams, liniments or patches as a carrier of the active ingredient. Such formulations can exhibit physiologically acceptable osmolarity by addition of pharmacologically acceptable buffers and salts. Such formulations may or may not, depending on the dispenser, contain preservatives such as benzalkonium chloride, chlorhexidine, chlorobutanol, parahydroxybenzoic acids and phenylmercuric salts such as nitrate, chloride, acetate, and borate, or antioxidants, as well as additives like EDTA, sorbitol, boric acid etc. as additives. Furthermore, particularly aqueous solutions may contain viscosity increasing agents such as polysaccharides, e.g., methylcellulose, mucopolysaccharides, e.g., hyaluronic acid and chondroitin sulfate, or polyalcohol, e.g., polyvinylalcohol. Various slow releasing gels and matrices may also be employed as well as soluble and insoluble ocular inserts, for instance, based on substances forming in-situ gels.

Dosing can be single dosage or cumulative (serial dosing), and can be readily determined by one skilled in the art. For instance, treating a condition disclosed in the present specification may comprise a one-time administration of an effective dose of a composition comprising a prostaglandin, prostamide, prostaglandin-glycerol ester, or analog thereof. As a non-limiting example, an effective dose of a composition comprising a prostaglandin, prostamide, prostaglandin-glycerol ester, or analog thereof can be administered once to an individual, e.g., as a single injection or deposition at or near the site exhibiting a condition or attribute associated with the condition. Alternatively, treatment of a cancer may comprise multiple administrations of an effective dose of a composition comprising a prostaglandin, prostamide, prostaglandin-glycerol ester, or analog thereof carried out over a range of time periods, such as, e.g., daily, once every few days, weekly, monthly or yearly. As a non-limiting example, a composition comprising a prostaglandin, prostamide, prostaglandin-glycerol ester, or analog thereof can be administered daily, once or twice weekly, once or twice monthly, or once or twice yearly to an individual. The timing of administration can vary from individual to individual, depending upon such factors as the severity of the condition. For example, an effective dose of a composition comprising a prostaglandin, prostamide, prostaglandin-glycerol ester, or analog thereof can be administered to an individual once a month for an indefinite period of time, or until the individual no longer requires therapy. To achieve the daily amount of medication depending on the formulation, the compositions disclosed in the present specification may be administered once or several times daily with or without antioxidants. A person of ordinary skill in the art will recognize that the condition of the individual can be monitored throughout the course of treatment and that the effective amount of a composition comprising a prostaglandin, prostamide, prostaglandin-glycerol ester, or analog thereof that is administered can be adjusted accordingly.

Thus, in an embodiment, a composition comprising a prostaglandin, prostamide, prostaglandin-glycerol ester, or analog thereof disclosed in the present specification is administered systemically to an individual. In another embodiment, a composition comprising a prostaglandin, prostamide, prostaglandin-glycerol ester, or analog thereof disclosed in the present specification is administered locally to an individual.

In another embodiment, the dose of a composition disclosed in the present specification that is administered to an epidermal region is in the range of about 0.1 ng to about 100 mg per day. In aspects of this embodiment, the dose of a composition disclosed in the present specification that is administered to an epidermal region is in the range of, e.g., about 1 ng to about 10 mg per day or about 10 ng to about 1 mg per day.

In certain embodiments, a composition comprising a prostaglandin, prostamide, prostaglandin-glycerol ester, or analog thereof disclosed in the present specification can be housed in containers suitable for dispensing the composition. The container can be a vial, bottle, tube, etc. In certain embodiments, the container will be a squeezable in order to release the composition therein. The container can have a lid, which may snap, twist, etc. on and off. The container should be such that the sterility of the composition therein is maintained. In certain embodiments, the container will have a safety seal prior to opening. In some embodiments, the container may hold from about 2 mL to about 10 mL of the composition. In other embodiments, the container may hold from 2 mL to about 5 mL of the composition. In certain embodiments, a maximum of 3 mL of the composition is disposed in the dispensing container.

Aspects of the present specification disclose, in part, a kit comprising a composition disclosed in the present specification. The kit can comprise a delivery system having one or more of an applicator brush, porous foam swab or pad, hollow tube, eye dropper, dipstick, or a combination thereof. In certain embodiments, the delivery system comprises a plurality of applicator brushes that have filaments coated with a lubricity enhancing agent. The lubricity enhancing agent can be a polymer that is coated onto the filaments in order to control the release of the composition from the brush, that is, the composition is not released from the brush until it makes contact with the eyebrow surface and the rate of release is such that a therapeutically appropriate amount of the composition is released from the brush onto the eyebrow surface. The applicator brushes of the kit are useful for applying the hair growth enhancing composition to the site of interest, that is, at least one eyebrow region. There may be a plurality of applicator brushes in a kit. For example, in a 30 day supply kit, there can be 60 applicators, such that there is one applicator for each eye, per application, for 30 days. Alternately, there can be 2, 10, 20, 30, 40, 50, 60, 90, 120, etc. applicators per kit. Within the kit, the applicator brushes may be packaged individually, or in sets of 2 or more. The applicator brushes are packaged such that they remain sterile until use. In certain embodiments, the applicator brushes can be packaged in plastic sheaths. Further, to prevent contamination of the eye, they are preferably single-use, disposable applicators.

The kit can also comprise a set of instructions. The instructions may include information useful to the end user such as how to use the delivery system and hair growth enhancing composition, how often to use it, etc.

The contents of the kit, the applicator brushes, container of eyebrow enhancing composition, and instructions, are enclosed in an outer casing. The outer casing can be a box, a sealed bag, a foil pouch, etc. In certain embodiments, the delivery system, container and instructions are enclosed in a box. In other embodiments of the kit, the container and instructions are contained in a first box, the delivery system is contained in a second box, and the first and second box are contained together in a third box.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

Aspect of the present specification can also be described as follows:
1. A method for treating alopecia in an individual in need thereof comprising the step of administering a therapeutically effective amount of a composition comprising a prostaglandin, prodrug thereof, salt thereof, or mixtures thereof to the individual, wherein the administration results in a reduction in alopecia.
2. A method for treating hair thinning in an individual in need thereof comprising the step of administering a therapeutically effective amount of a composition comprising a prostaglandin, prodrug thereof, salt thereof, or mixtures thereof to the individual, wherein the administration results in a reduction in hair thinning.
3. A method for treating hair color loss in an individual in need thereof comprising the step of administering a therapeutically effective amount of a composition comprising a prostaglandin, prodrug thereof, salt thereof, or mixtures thereof to the individual, wherein the administration results in a reduction in hair color loss.
4. A method for treating an attribute associated with hair loss in an individual in need thereof comprising the step of administering a therapeutically effective amount of a composition comprising a prostaglandin, prodrug thereof, salt thereof, or mixtures thereof to the individual, wherein the administration results in a reduction in the attribute associated with hair loss.
5. A method for treating an attribute associated with hair thinning in an individual in need thereof comprising the step of administering a therapeutically effective amount of a composition comprising a prostaglandin, prodrug thereof, salt thereof, or mixtures thereof to the individual, wherein the administration results in a reduction in the attribute associated with hair thinning.
6. A method for treating an attribute associated with hair color loss in an individual in need thereof comprising the step of administering a therapeutically effective amount of a composition comprising a prostaglandin, prodrug thereof, salt thereof, or mixtures thereof to the individual, wherein the administration results in a reduction in the attribute associated with hair color loss.
7. The method of 1-6, wherein the prostaglandin is a PGD₂, a PGE₂, a PGF_{2α}, a 11β-PGF_{2α}, a PGG₂, a PGH₂, a PGI₂, a prostacyclin, or a thromboxane A₂.
8. The method of 1, wherein the alopecia is scarring alopecia or non-scarring alopecia.
9. The method of 8, wherein the scarring alopecia is associated with a bullous disease, a chemical exposure, a discoid lupus erythematosus, a severre folliculitis, a lichen planopilaris, a dissecting cellulitis, a central centrifugal cicatricial alopecia, a postmenopausal frontal fibrosing alopecia, a tumor, or a skin outgrowth.
10. The method of 8, wherein the non-scarring alopecia is anagen effluvium, alopecia adnata, alopecia androgenetica, alopecia areata, alopecia congenitalis, alopecia diffusa, alopecia disseminate, alopecia follicularis, alopecia leprotica, alopecia marginalis, alopecia medicamentosa, alopecia mucinosa, alopecia neurotica, alopecia pityrodes, alopecia presenili, alopecia senilis, alopecia symptomatica, alopecia syphilitica, alopecia totalis, alopecia toxica, alopecia triangularis, alopecia triangularis congenitalis, alopecia universalis, folliculitis, olliculitis decalvans, traction alopecia, trichotillomania, telogen effluvium, or inherited disorder of the hair shaft.
11. The method of 4, wherein the attribute associated with hair loss is no new hair shaft growth, reduced rate of hair shaft growth, reduced hair shaft diameter (thickness), reduced hair shaft length, reduced hair density, reduced hair pigmentation, reduced hair shaft luster, reduced hair health, reduced time a hair follicle spends in anagen phase, reduced time a hair follicle spends in catagen phase, reduced time a hair follicle spends in telogen phase, premature release of hair shaft from hair follicle, premature initiation of apoptosis in hair follicle, premature conversion of a terminal hair into a vellus hair.
12. The method of 4, wherein reduction of the attribute associated with hair loss is by increasing the rate of hair growth, increasing hair thickness, increasing hair length, increasing hair density, increasing number of hairs produce per follicle, increasing hair pigmentation, increasing hair luster, converting intermediate or vellus hair to terminal hair, increasing hair health, increasing the time a hair follicle remains in anagen phase, increasing the time a hair follicle remains in catagen phase, increasing the time a hair follicle remains in telogen phase, prolonging or preventing the release of the hair shaft from the hair follicle, or prolonging or preventing the initiation of apoptosis in a hair follicle.
13. The method of 5, wherein the attribute associated with hair thinning is decreased hair length, decreased hair diameter, decreased keratinization of the hair shaft, increased fragility of the hair shaft, reduced hair health, reduced time a hair follicle spends in anagen phase, reduced time a hair follicle spends in catagen phase, reduced time a hair follicle spends in telogen phase, or premature conversion of a terminal hair into a vellus hair.
14. The method of 5, wherein reduction of the attribute associated with hair thinning is by increasing the rate of hair growth, increasing hair thickness, increasing hair length, increasing hair density, increasing number of hairs produce per follicle, increasing hair pigmentation, increasing hair luster, increasing keratin production, converting intermediate or vellus hair to terminal hair, increasing hair health, increasing the time a hair follicle remains in anagen phase, increasing the time a hair follicle remains in catagen phase, or increasing the time a hair follicle remains in telogen phase.
15. The method of 6, wherein the attribute associated with hair color loss is decreased pigmentation of the hair shaft, decreased melanin production, increased death of melanocytes.
16. The method of 6, wherein reduction of the attribute associated with hair color loss is by increasing pigmentation of the hair shaft, increasing melanin production, increasing hair luster, converting intermediate or vellus hair to terminal hair, increasing hair health, increasing the time a hair follicle remains in anagen phase, increasing the time a hair follicle remains in catagen phase, increasing the time a hair follicle remains in telogen phase, prolonging or preventing melanocyte death, or prolonging or preventing the initiation of apoptosis in a hair follicle.

Other aspect of the present specification can also be described as follows:
1. A method for treating alopecia in an individual in need thereof comprising the step of administering a therapeutically effective amount of a composition comprising a prostamide, prodrug thereof, salt thereof, or mixtures thereof to the individual, wherein the administration results in a reduction in alopecia.
2. A method for treating hair thinning in an individual in need thereof comprising the step of administering a therapeutically effective amount of a composition comprising prostamide, prodrug thereof, salt thereof, or mixtures thereof to the individual, wherein the administration results in hair thinning.
3. A method for treating hair color loss in an individual in need thereof comprising the step of administering a therapeutically effective amount of a composition comprising a prostamide, prodrug thereof, salt thereof, or mixtures thereof to the individual, wherein the administration results in a reduction in hair color loss.
4. A method for treating an attribute associated with hair loss in an individual in need thereof comprising the step of administering a therapeutically effective amount of a composition comprising a prostamide, prodrug thereof, salt thereof, or mixtures thereof to the individual, wherein the administration results in a reduction in the attribute associated with hair loss, hair thinning, or hair color loss.
5. A method for treating an attribute associated with hair thinning in an individual in need thereof comprising the step of administering a therapeutically effective amount of a composition comprising a prostamide, prodrug thereof, salt thereof, or mixtures thereof to the individual, wherein the administration results in a reduction in the attribute associated with hair thinning.
6. A method for treating an attribute associated with hair color loss in an individual in need thereof comprising the step of administering a therapeutically effective amount of a composition comprising a prostamide, prodrug thereof, salt thereof, or mixtures thereof to the individual, wherein the administration results in a reduction in the attribute associated with hair color loss.
7. The method of 1-6, wherein the prostamide is a prostamide D₂, prostamide E₂, a prostamide F_{2α}, a 11β-prostamide F_{2α}, a prostamide G₂, a prostamide H₂, or a prostamide I₂.
8. The method of 1, wherein the alopecia is scarring alopecia or non-scarring alopecia.
9. The method of 8, wherein the scarring alopecia is associated with a bullous disease, a chemical exposure, a discoid lupus erythematosus, a severre folliculitis, a lichen planopilaris, a dissecting cellulitis, a central centrifugal cicatricial alopecia, a postmenopausal frontal fibrosing alopecia, a tumor, or a skin outgrowth.
10. The method of 8, wherein the non-scarring alopecia is anagen effluvium, alopecia adnata, alopecia androgenetica, alopecia areata, alopecia congenitalis, alopecia diffusa, alopecia disseminate, alopecia follicularis, alopecia leprotica, alopecia marginalis, alopecia medicamentosa, alopecia mucinosa, alopecia neurotica, alopecia pityrodes, alopecia presenili, alopecia senilis, alopecia symptomatica, alopecia syphilitica, alopecia totalis, alopecia toxica, alopecia triangularis, alopecia triangularis congenitalis, alopecia universalis, folliculitis, olliculitis decalvans, traction alopecia, trichotillomania, telogen effluvium, or inherited disorder of the hair shaft.
11. The method of 4, wherein the attribute associated with hair loss is no new hair shaft growth, reduced rate of hair shaft growth, reduced hair shaft diameter (thickness), reduced hair shaft length, reduced hair density, reduced hair pigmentation, reduced hair shaft luster, reduced hair health, reduced time a hair follicle spends in anagen phase, reduced time a hair follicle spends in catagen phase, reduced time a hair follicle spends in telogen phase, premature release of hair shaft from hair follicle, premature initiation of apoptosis in hair follicle, premature conversion of a terminal hair into a vellus hair.
12. The method of 4, wherein reduction of the attribute associated with hair loss is by increasing the rate of hair growth, increasing hair thickness, increasing hair length, increasing hair density, increasing number of hairs produce per follicle, increasing hair pigmentation, increasing hair luster, converting intermediate or vellus hair to terminal hair, increasing hair health, increasing the time a hair follicle remains in anagen phase, increasing the time a hair follicle remains in catagen phase, increasing the time a hair follicle remains in telogen phase, prolonging or preventing the release of the hair shaft from the hair follicle, or prolonging or preventing the initiation of apoptosis in a hair follicle.
13. The method of 5, wherein the attribute associated with hair thinning is decreased hair length, decreased hair diameter, decreased keratinization of the hair shaft, increased fragility, reduced hair health, reduced time a hair follicle spends in anagen phase, reduced time a hair follicle spends in catagen phase, reduced time a hair follicle spends in telogen phase, or premature conversion of a terminal hair into a vellus hair.
14. The method of 5, wherein reduction of the attribute associated with hair thinning is by increasing the rate of hair growth, increasing hair thickness, increasing hair length, increasing hair density, increasing number of hairs produce per follicle, increasing hair pigmentation, increasing hair luster, increasing keratin production, converting intermediate or vellus hair to terminal hair, increasing hair health, increasing the time a hair follicle remains in anagen phase, increasing the time a hair follicle remains in catagen phase, or increasing the time a hair follicle remains in telogen phase.
15. The method of 6, wherein the attribute associated with hair color loss is decreased pigmentation of the hair shaft, decreased melanin production, increased death of melanocytes.
16. The method of 6, wherein reduction of the attribute associated with hair color loss is by increasing pigmentation of the hair shaft, increasing melanin production, increasing hair luster, converting intermediate or vellus hair to terminal hair, increasing hair health, increasing the time a hair follicle remains in anagen phase, increasing the time a hair follicle remains in catagen phase, increasing the time a hair follicle remains in telogen phase, prolonging or preventing melanocyte death, or prolonging or preventing the initiation of apoptosis in a hair follicle.

Other aspect of the present specification can also be described as follows:
1. A method for treating alopecia in an individual in need thereof comprising the step of administering a therapeutically effective amount of a composition comprising a prostaglandin-glycerol ester, prodrug thereof, salt thereof, or mixtures thereof to the individual, wherein the administration results in a reduction in alopecia.
2. A method for treating hair thinning in an individual in need thereof comprising the step of administering a therapeutically effective amount of a composition comprising a prostaglandin-glycerol ester, prodrug thereof, salt thereof, or mixtures thereof to the individual, wherein the administration results in a reduction in hair thinning.
3. A method for treating hair color loss in an individual in need thereof comprising the step of administering a therapeutically effective amount of a composition comprising a prostaglandin-glycerol ester, prodrug thereof, salt thereof, or mixtures thereof to the individual, wherein the administration results in a reduction in hair color loss.
4. A method for treating an attribute associated with hair loss in an individual in need thereof comprising the step of administering a therapeutically effective amount of a composition comprising a prostaglandin-glycerol ester, prodrug thereof, salt thereof, or mixtures thereof to the individual, wherein the administration results in a reduction in the attribute associated with hair loss, hair thinning, or hair color loss.
5. A method for treating an attribute associated with hair thinning in an individual in need thereof comprising the step of administering a therapeutically effective amount of a composition comprising a prostaglandin-glycerol ester, prodrug thereof, salt thereof, or mixtures thereof to the individual, wherein the administration results in a reduction in the attribute associated with hair thinning.
6. A method for treating an attribute associated with hair color loss in an individual in need thereof comprising the step of administering a therapeutically effective amount of a composition comprising a prostaglandin-glycerol ester, prodrug thereof, salt thereof, or mixtures thereof to the individual, wherein the administration results in a reduction in the attribute associated with hair color loss.
7. The method of 1-6, wherein the prostaglandin-glycerol ester is a PGD₂-glycerol ester, a PGE₂-glycerol ester, a PGF_{2α}-glycerol ester, a 11β-PGF_{2α}-glycerol ester, a PGG₂-glycerol ester, a PGH₂-glycerol ester, a PGI₂-glycerol ester, a prostacyclin-glycerol ester, or a thromboxane A₂-glycerol ester.
8. The method of 1, wherein the alopecia is scarring alopecia or non-scarring alopecia.
9. The method of 8, wherein the scarring alopecia is associated with a bullous disease, a chemical exposure, a discoid lupus erythematosus, a severre folliculitis, a lichen planopilaris, a dissecting cellulitis, a central centrifugal cicatricial alopecia, a postmenopausal frontal fibrosing alopecia, a tumor, or a skin outgrowth.
10. The method of 8, wherein the non-scarring alopecia is anagen effluvium, alopecia adnata, alopecia androgenetica, alopecia areata, alopecia congenitalis, alopecia diffusa, alopecia disseminate, alopecia follicularis, alopecia leprotica, alopecia marginalis, alopecia medicamentosa, alopecia mucinosa, alopecia neurotica, alopecia pityrodes, alopecia presenili, alopecia senilis, alopecia symptomatica, alopecia syphilitica, alopecia totalis, alopecia toxica, alopecia triangularis, alopecia triangularis congenitalis, alopecia universalis, folliculitis, olliculitis decalvans, traction alopecia, trichotillomania, telogen effluvium, or inherited disorder of the hair shaft.
11. The method of 4, wherein the attribute associated with hair loss is no new hair shaft growth, reduced rate of hair shaft growth, reduced hair shaft diameter (thickness), reduced hair shaft length, reduced hair density, reduced hair pigmentation, reduced hair shaft luster, reduced hair health, reduced time a hair follicle spends in anagen phase, reduced time a hair follicle spends in catagen phase, reduced time a hair follicle spends in telogen phase, premature release of hair shaft from hair follicle, premature initiation of apoptosis in hair follicle, premature conversion of a terminal hair into a vellus hair.
12. The method of 4, wherein reduction of the attribute associated with hair loss is by increasing the rate of hair growth, increasing hair thickness, increasing hair length, increasing hair density, increasing number of hairs produce per follicle, increasing hair pigmentation, increasing hair luster, converting intermediate or vellus hair to terminal hair, increasing hair health, increasing the time a hair follicle remains in anagen phase, increasing the time a hair follicle remains in catagen phase, increasing the time a hair follicle remains in telogen phase, prolonging or preventing the release of the hair shaft from the hair follicle, or prolonging or preventing the initiation of apoptosis in a hair follicle.
13. The method of 5, wherein the attribute associated with hair thinning is decreased hair length, decreased hair diameter, decreased keratinization of the hair shaft, increased fragility, reduced hair health, reduced time a hair follicle spends in anagen phase, reduced time a hair follicle spends in catagen phase, reduced time a hair follicle spends in telogen phase, or premature conversion of a terminal hair into a vellus hair.
14. The method of 5, wherein reduction of the attribute associated with hair thinning is by increasing the rate of hair growth, increasing hair thickness, increasing hair length, increasing hair density, increasing number of hairs produce per follicle, increasing hair pigmentation, increasing hair luster, increasing keratin production, converting intermediate or vellus hair to terminal hair, increasing hair health, increasing the time a hair follicle remains in anagen phase, increasing the time a hair follicle remains in catagen phase, or increasing the time a hair follicle remains in telogen phase.
15. The method of 6, wherein the attribute associated with hair color loss is decreased pigmentation of the hair shaft, decreased melanin production, increased death of melanocytes.
16. The method of 6, wherein reduction of the attribute associated with hair color loss is by increasing pigmentation of the hair shaft, increasing melanin production, increasing hair luster, converting intermediate or vellus hair to terminal hair, increasing hair health, increasing the time a hair follicle remains in anagen phase, increasing the time a hair follicle remains in catagen phase, increasing the time a hair follicle remains in telogen phase, prolonging or preventing melanocyte death, or prolonging or preventing the initiation of apoptosis in a hair follicle.

### EXAMPLES

### Example 1

### Ocularly Applied Bimatoprost 0.03% Increased Eyelash Growth

To assess the safety, efficacy, and subjective experience of using dermal application of bimatoprost 0.03% for the growth of natural eyelashes and as described herein hair in at least one eyebrow region, a prospective, open-label study of subjects who desired longer, thicker (fuller), and darker natural eyelashes was conducted.

This was a prospective, open-label study of subjects who wanted to grow more prominent eyelashes. Subjects were all women, were at least 18 years old, and had an intraocular pressure no higher than 22 mm Hg. The protocol was in compliance with the Declaration of Helsinki and in accordance with applicable Institutional Review Board regulations approved Aug. 28, 2005. Study subjects gave informed consent prior to initiation of any study-related procedures.

Exclusion criteria included previous diagnosis of glaucoma or ocular hypertension and hypersensitivity to bimatoprost or any component of the study treatment. Subjects were also excluded if they had an uncontrolled systemic disease or a history of ocular surgery within the 3 months prior to baseline. Subjects of childbearing potential who were pregnant, lactating, planning a pregnancy or not using a reliable form of birth control were also excluded.

Subjects were instructed to apply bimatoprost 0.03% to the eyelid lash line once daily for 12 weeks using an applicator (Studio Basics Professional Tools Eye Applicators, Paris Presents Incorporated, Gumee, Ill.) for each upper eyelid each evening and, if necessary, to wipe the areas immediately around the eyelid afterward to remove excess medication. Periodic use of artificial tears was allowed, but only prior to administration of bimatoprost 0.03%.

Clinical Examinations. Assessments were conducted at baseline and at weekly study visits at weeks 1, 4, 8, 12 (end of treatment period), and 16 (post-treatment follow-up period). At each visit, subjects underwent a complete eye examination by an ophthalmologist that included intraocular pressure measurements, visual acuity, and biomicroscopy. Subjects were queried at each visit for adverse events and these were recorded by the investigator. The presence of periorbital darkening was noted and graded on a scale of 0 to 4, and adverse event data were collected at each visit during the treatment period.

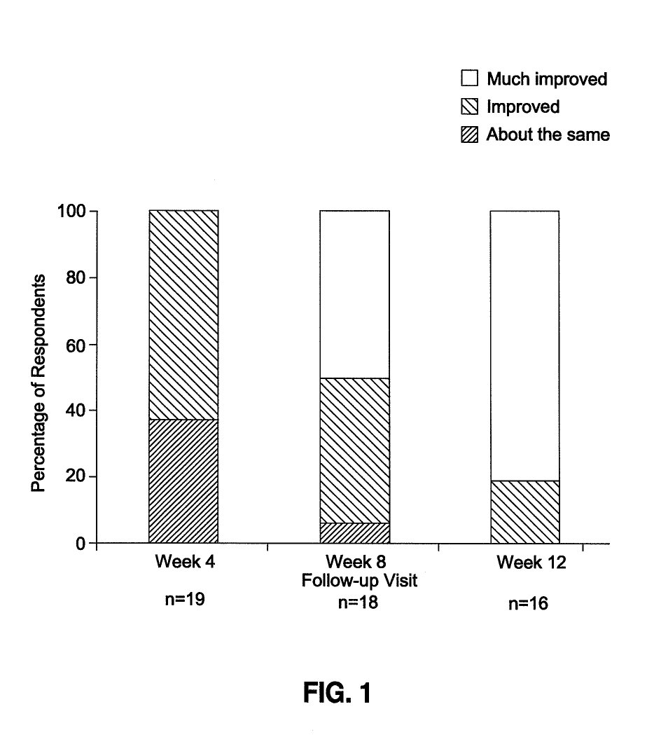

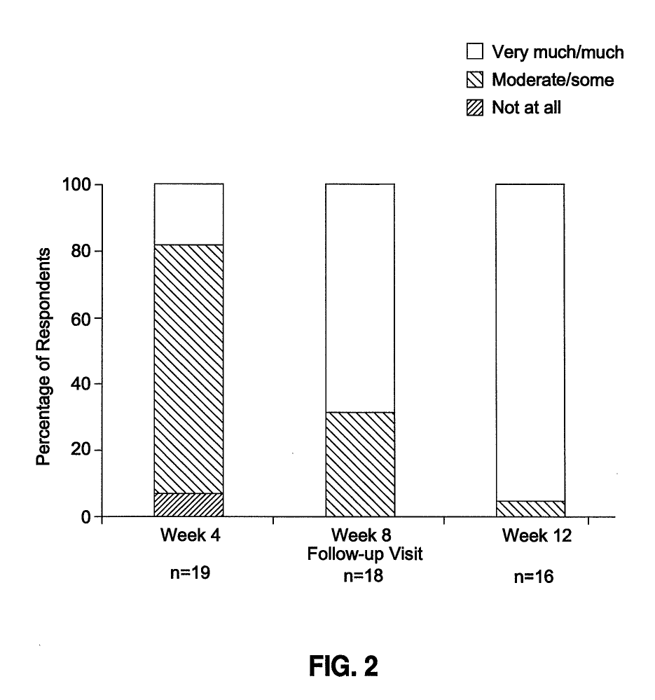

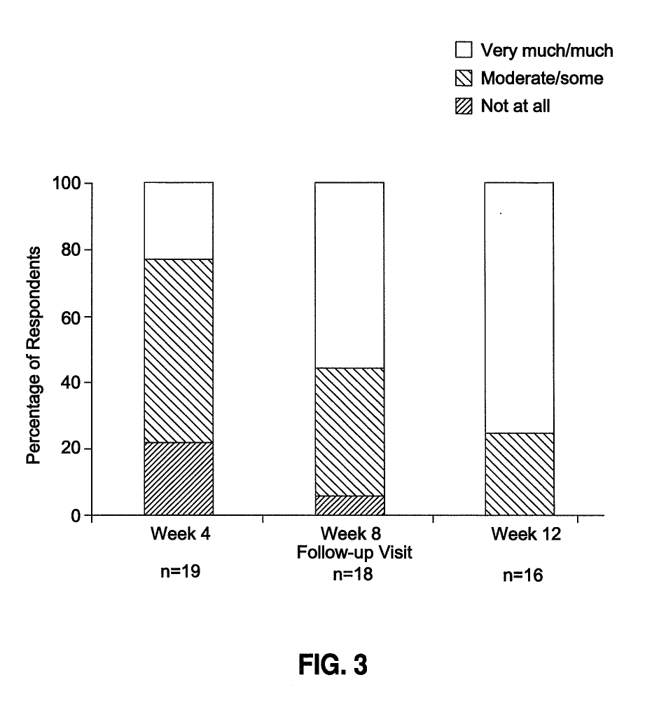

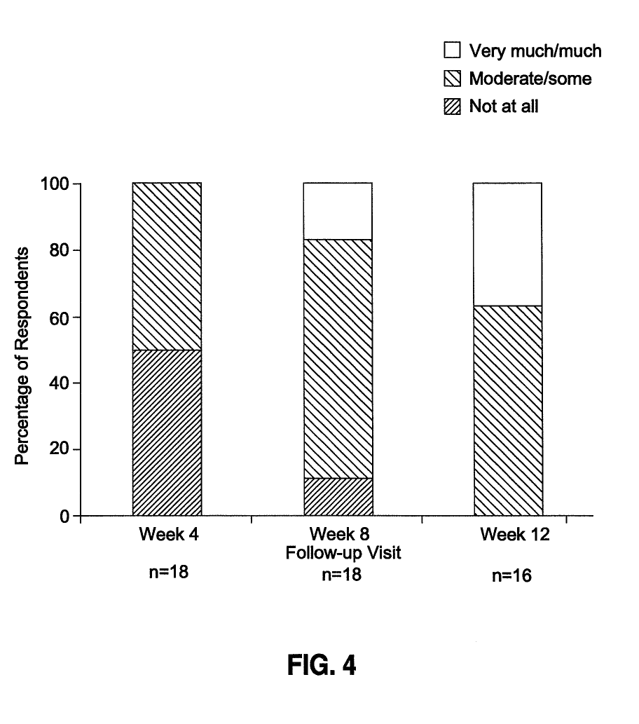

Health Outcomes Questionnaires. A questionnaire was used to track adverse events and to assess the subjects' satisfaction with treatment and their subjective evaluation of their eyelashes' appearance. Subjects were asked to answer all questions and return the questionnaire at weeks 4, 8, and 12. Subjects were also asked to respond verbally to 2 additional questions and their responses were recorded by site staff (FIGS. 1-4).

Photographic Documentation and Analysis. Frontal—(with eyes open) and side-view photographs were taken of the subjects' eyes at each visit; frontal-view images with eyes closed were taken starting at week 1. The photographic assessment planned as a primary a priori endpoint was a dynamic measure assessing change at each visit from baseline photos. The dynamic scale of global change in increased length, thickness, pigmentation, and number of lashes was +0=no change, +½=little change, +1=mild noticeable change, +2=moderate notable change, and +3=major very notable change. A static photographic assessment was calculated. Photographs from subjects who had frontal eyes-closed images available from weeks 1 and 12 were evaluated in a post hoc analysis and scored independently by 2 raters using a validated Global Eyelash Assessment (GEA) scale, designed to objectively evaluate the quality of a subject's eyelashes. One evaluator was a physician and the other was a health outcomes researcher employed by the study sponsor. In this clinician-graded assessment, overall eyelash prominence was rated on a scale from 1 (minimal) to 4 (very marked). The two independently assessed scores for each photograph (1 from each evaluator) were averaged and the change from week 1 was calculated for each patient.

Statistical Analyses. A 2-sided paired t-test was used to evaluate the intraocular pressure change from baseline, and the GEA change from week 1 to week 12 was compared using a Wilcoxon signed-rank test. Analysis of intraocular pressure was performed on a per-eye and per-subject basis. A significance level of 0.05 was used for all statistical tests. Categorical variables were summarized with frequency, count, and percentages.

Results. Twenty-nine women enrolled in the study, but 1 withdrew consent prior to dosing; thus, 28 were included in the analyses. Subjects ranged in age from 32 to 73 years, with a mean age of 48.9 years. At total of 39.3% of subjects were between the ages of 45 and 54 years. One subject was Asian and all the others were Caucasian. Eleven subjects had brown irides, 7 had hazel, 7 had blue, and 3 had green. Based on the medical histories collected for general and ophthalmic disorders, all subjects were in good general health. Dry eye was the most commonly reported pre-existing ophthalmic disorder ( 6/28; 21.4%).

Of the 28 subjects, 24, 25, 21, and 24 returned for the week 1, 4, 8, and 12 follow-up visits, respectively. Twenty-two subjects returned for the final visit (week 16), 1 month after bimatoprost 0.03% treatment ended. Two of the 28 subjects did not return for any follow-up visits. Not all attendees at each follow-up visit returned a questionnaire, and each question was not always answered. Relevant response rates are indicated for each item described below.

Intraocular Pressure Results. At baseline, the mean intraocular pressure (±SD) was 13.75±2.82 mm Hg. The mean change from baseline in intraocular pressure across all weeks ranged from +0.98 to −0.79 (per subject) or +0.98 to −0.81 (per eye). The per-subject changes from baseline in intraocular pressure at each study visit were not statistically significant at any time point. The per-eye analysis of intraocular pressure measurement change from baseline was statistically significant at weeks 1 (−0.81±2.72 mm Hg; P=0.0470) and 4 (0.98±3.23; P=0.0412), but not at any other follow-up visit. The mean change from baseline intraocular pressure was less than 1 mm Hg at each time point. The changes in intraocular pressure were within the accepted variability of the test and normal intraocular pressure variation and were deemed not clinically significant.

Adverse events results. The adverse events reported (using verbatim terminology) are shown in Table 2. None of the adverse events caused withdrawal from the study and no serious or unexpected adverse events were reported. Periorbital darkening was noted for 5 of 28 (18%) subjects during the course of the study. For 3 of these 28 subjects (11%), however, the pigmentation changes were noted as “possible,” “slight,” or “a little.” Hyperemia was not observed on biomicroscopy examination for any subject at any visit. No adverse events related to visual acuity or intraocular pressure were reported.

Questionnaires and Verbal Responses. In response to the question “Do your eyes sting or burn upon application?.” 10 respondents indicated “yes” at least once: 42.2% ( 8/19) at week 4, 33.3% ( 6/18) at week 8, and 26.7% ( 4/15) at week 12.

Patient-reported effectiveness was also evaluated using questionnaires. Among the 16 respondents at the end of the treatment period (week 12), most (56.3%; 9/16) indicated that they had started to notice changes in their eyelashes by week 8. One subject reported that she noticed changes in her eyelashes within the first week, 3 subjects noticed changes within the first month, and 3 subjects noticed changes by week 12. No respondent indicated that her eyelashes did not change. Twelve of the 16 respondents at week 12 (81.3%) chose “longer” as the eyelash change they noticed most, 1 subject indicated both longer and thicker, and 3 subjects reported all 3 options—longer, thicker, and darker.

At weeks 4, 8, and 12, when questionnaires were distributed, all respondents (16, 15, and 15, respectively) indicated that satisfaction or activity limitations/symptoms/emotions/overall quality of life related to their eyelashes either had improved or was the same relative to before they started treatment. At each of these visits, the majority of respondents agreed that bimatoprost 0.03% was easier to use than daily mascara.

The proportion of respondents indicating that their eyelashes were “much improved” compared with before starting treatment increased over the treatment period, and by week 12 all 16 respondents indicated that their eyelashes were “improved” or “much improved”. By week 12, most respondents agreed “very much/much” that the treatment was helpful (94%; 15/16) and that they had done something positive for their appearance (75%; 12/16). All respondents agreed at least somewhat that they felt more attractive at the end of the treatment period.

The verbal open-ended questions prompted a variety of responses. At the week 4 visit, 15 of 24 respondents (56.0%) indicated that they had noticed some growth or darkening of their eyelashes. At week 12, all 23 respondents (100%) responded affirmatively that they had noticed growth or darkening of their eyelashes.

Photographic Assessment. Frontal-view photographs taken at weeks 1 and 12 were available for 19 subjects. At week 1, the majority of subjects (79.0%; 15/19) were rated as 2 (moderate). At week 12, the majority of subjects (94.7%; 18/19) were rated 3 (marked) or higher. For all 19 subjects included in this assessment, the mean week-1 GEA score was 2.0±0.47 and the mean change from week 1 to week 12 was 1.37±0.44 (P<0.0001). For the 17 subjects who had week-1 GEA scores of 1 or 2, the mean GEA score at week 1 was 1.88±0.33. At week 12, the mean increase in GEA score for these subjects was 1.41±0.44 (P<0.0001).

Conclusion. Subjects in this study observed that bimatoprost 0.03% applied to the eyelid margin improved their appearance by increasing the length, thickness (fullness), and darkness of their eyelashes. Treatment was associated with a few mild adverse effects, and although some variation in mean intraocular pressure was observed, it did not change by more than 1 mm Hg, suggesting that changes were not clinically significant. Bimatoprost 0.03% has a proven safety record with ocular instillation, and this study shows that it is also safe for eyelid margin application and would be applicable to the eyebrow region as well. Controlled application of bimatoprost to the eyelid margin line positively affects the appearance of the eyelashes, with users reporting increased length, thickness (fullness), and darkness of lashes.

TABLE 2
Adverse events.

| Time point | Adverse Event | Subjects Reported (n) |
|---|---|---|
| Week 1 | Eye redness | 2 |
| (n = 24) | Possible brown eye shadow effect | 1 |
| | Periorbital pigmentation changes | 0 |
| Week 4 | Eye redness | 4 |
| (n = 27) | Mild itchiness | 3 |
| | Burning | 1 |
| | Dryness and tightness | 1 |
| | Periorbital redness | 1 |
| | Periorbital pigmentation changes | 3 |
| Week 8 | Mild itchiness | 2 |
| (n = 20) | Burning | 2 |
| | Redness and dryness | 1 |
| | Periorbital pigmentation changes | 1 |
| Week 12 | Eye redness | 1 |
| (n = 23) | Red and sore | 1 |
| | Eyelid redness | 1 |
| | Itching | 1 |
| | Dryness and darkening | 1 |
| | Periorbital pigmentation changes | 3 |

### Example 2

### Additional Study Showing that Bimatoprost 0.03% Leads to Increased Hair Growth

The objective of this study was to evaluate the safety and efficacy of bimatoprost 0.03% solution once daily compared with vehicle in increasing overall eyelash prominence following dermal administration to the upper eyelid margins.

This study consisted of 8 visits: screening (day −14 to −1); baseline (day 1); week 1; months 1, 2, 3, and 4 (or early exit); and month 5 (post treatment follow-up). Treatment was initiated on day 1 and concluded at month 4 (week 16), after which there was a post-treatment follow-up period lasting 1 month.

The primary clinical hypothesis tested in this study was that bimatoprost 0.03% solution is more effective than vehicle in increasing overall eyelash prominence as measured by the difference between the 2 groups in the proportion of subjects at month 4 (week 16) with at least a 1-grade increase from baseline in the 4-point global eyelash assessment (GEA) score.

During the screening visit, each subject was assessed for inclusion/exclusion criteria. Qualifying subjects were randomly allocated in a 1:1 ratio to treatment with bimatoprost 0.03% solution or vehicle, to be applied to both upper eyelid margins once daily in the evening for 4 months.

At screening and at each follow-up visit, each subject was to complete 1 to 4 patient reported outcomes (PRO) questionnaires before any study procedures took place. The PRO questionnaires addressed subjects' satisfaction with their eyelashes with regard to length, fullness/thickness, and color/intensity, and any perceived changes in these characteristics since the initiation of study treatment. Visual acuity, IOP, and biomicroscopy were measured at every visit except baseline (day 1). Subjects' eyelashes were photographed at every visit from both superior (45° of both eyes closed) and frontal (0° of both eyes open) views under standardized lighting conditions, using standardized digital photography equipment provided by Canfield Scientific, Inc (Fairfield, N.J.).

Subjects were considered to have completed the study when all visit procedures were completed at month 5. Subjects were considered to have exited the study when the early exit visit was completed at any time prior to month 5 for any reason.

This study used a reliable and reproducible measure of overall eyelash prominence (ie, the GEA score) as a primary efficacy measure. The GEA was assessed as an efficacy measure for this study by conducting a single-center study with 68 subjects and 7 raters evaluating overall eyelash prominence using the GEA with photonumeric guide. On the same day, each rater evaluated each subject 2 times at least 1 hour apart in order to assess the intra and inter-rater reliability. Results of this study demonstrated that the GEA scale with photonumeric guide can be considered to be a reliable, reproducible instrument in grading overall eyelash prominence. Four months of treatment was chosen for this study to assess safety over a longer period of treatment.

For enrollment into the study, each subject had to meet all of the following inclusion criteria: male or female, at least 18 years of age, dissatisfied with their overall eyelash prominence, written informed consent and authorization obtained prior to any study-related procedures, screening and baseline GEA score of a 1 or 2, a best-corrected visual acuity score equivalent to a Snellen acuity of 20/100 or better in each eye, using a logarithmic acuity chart for testing at 10 feet, IOP≦20 mm Hg in each eye standardized eyelash photographs at the screening visit of acceptable quality for image analysis as verified by Canfield Scientific, Inc., ability to follow study instructions and willingness to complete all required procedures and visits.

Bimatoprost 0.03% sterile solution contained 0.3 mg/mL of bimatoprost, sodium phosphate dibasic heptahydrate, sodium chloride, citric acid monohydrate, hydrochloric acid, sodium hydroxide, benzalkonium chloride 0.005% and purified water.

Bimatoprost vehicle sterile solution contained sodium phosphate dibasic heptahydrate, sodium chloride, citric acid monohydrate, hydrochloric acid, sodium hydroxide, benzalkonium chloride 0.005% and purified water. Study medications were to be stored at room temperature at all times.

Subjects applied study medication to the upper eyelid margins using the supplied, disposable, single-use-per-eye applicator once daily in the evening for 4 months. Subjects were instructed to dab or blot any excess study medication runoff on the area outside the upper eyelash margin with a tissue or other absorbent cloth; the upper lid margin was to feel evenly and lightly moist without runoff. Subjects were instructed not to apply study medication to the lower eyelash line. Subjects were to apply the treatment to a clean face after all makeup had been removed and before any other facial care products were applied (eg, lotion). Subjects who wear contact lenses were to remove them before application of study medication and were not to reinsert them for at least 30 minutes.

The primary efficacy measurement for this study was the subject's overall (ie, both eyes scored together, superior and frontal views) eyelash prominence at month 4 (week 16) as measured by the investigator using the GEA scale. The GEA is a 4-point scale with a photonumeric guide which uses the following scores. GEA Score Description of Eyelash Prominence: 1 Minimal (includes everything up to minimal [includes worst possible/none]); Corresponding to photoguide grade 1 frontal and superior views; 2 Moderate; Corresponding to photoguide grade 2 frontal and superior views; 3 Marked; Corresponding to photoguide grade 3 frontal and superior views; 4 Very Marked (includes very marked and above [includes best possible]); Corresponding to photoguide grade 4 frontal and superior views.

The primary efficacy variable was the change in GEA score from the baseline measurement to the month 4 (week 16) measurement. A clinical success was defined as at least a 1-grade increase from baseline. Secondary efficacy measurements collected in this study included eyelash length, progressive eyelash thickness/fullness, and eyelash darkness (intensity), each determined by image analysis of digital eyelash photographs (superior view) across both eyes. The digital image analysis was based on standardized equipment and subject preparation. Digital image analysis is a photographic process developed and performed by Canfield Scientific, Inc. The details regarding these processes are maintained by Canfield Scientific, Inc. and are available upon request. The information describing software and technical processes of digital image analysis is maintained in standard operating procedures (SOPs) and work instruction manuals on file at Canfield Scientific, Inc. Upper eyelash length was measured within a defined eyelash boundary for each eye, known as the full area of interest (AOI). For the digital image, the computer software divided the full AOI image into a series of 25 vertical pixel segments. Within each segment, the maximum upper eyelash length (defined as the maximum height of each segment) was measured in pixels. The mean number of pixels over all segments represented the upper eyelash length and was computed for each digital image across both eyes. Upper eyelash length was additionally measured in terms of millimeters (mm). The principal variable for eyelash length was change from baseline within the full AOI in pixels. Upper eyelash thickness/fullness was measured within 3 preset rectangular areas (proximal, medial, and distal, each 300×25 pixels) positioned at fixed distances from a standardized point on the eyelash margin. For each superior-view image, the number of pixels representing the upper eyelashes was counted within each preset rectangular area. Eyelash thickness/fullness was assessed across both eyes as an average of the 3 rectangular areas (ie, average progressive eyelash thickness), individually for the 3 areas (proximal, medial, and distal), within the full AOI, and within the spline (a narrow area approximately 5 pixels wide, bisecting the AOI). Upper eyelash thickness/fullness was additionally measured in terms of mm2. The principal variable for eyelash thickness/fullness was change from baseline in average progressive eyelash thickness, expressed in pixels as percent of AOI. Upper eyelash darkness was determined by lash intensity of the upper eyelash area within the spline. Darkness (intensity) of each pixel blob (a continuous collection of pixels that are touching) was reported as mean intensity of the red, green, and blue scale. The mean intensity of each pixel blob was then interpreted on an 8-bit image grayscale on the continuum of 0 (black) and 255 (white). The mean lash intensity was the average intensities of all pixel blobs and was a measure of upper eyelash darkness. Eyelash intensity was calculated within the full AOI and within the spline. The principal analysis variable for eyelash intensity was change from baseline within the spline.

Four PRO questionnaires were collected during this study. PRO questionnaire 1, collected at every study visit, was a static measure of satisfaction with regard to subjects' eyelashes and the study treatment. Subjects were asked to answer using the 5-point scale presented for each question (eg, very satisfied, satisfied, neutral, unsatisfied, very unsatisfied). Satisfaction was assessed by analysis of the change from baseline for 23 individual items and by analyses of 3 domains. Domain 1 (8 questions) assessed the subjects' satisfaction with physical attributes of eyelashes including length, fullness/thickness, and overall satisfaction with eyelashes. Domain 2 (10 questions) assessed subjects' satisfaction with subjective attributes of eyelashes such as they relate to feelings of confidence, professionalism, and attractiveness. Domain 3 (5 questions) assessed subjects' satisfaction with their daily routine with regard to the amount of time spent on the application and removal of mascara, and the hassle of making eyelashes presentable. For questions within domains 1 and 2, a lower score represented higher satisfaction (ie, the minimum score translated to “no impairment of life quality” and the maximum score, “maximum impairment”); for domain 3, a higher score represented higher satisfaction. PRO questionnaire 2, collected only during the day 1 visit, asked the subjects which effects of eyelash enhancement were most valuable among eyelash length, fullness/thickness, darkness, and number of eyelashes. Subjects were asked to rate the importance of each using a 5-point scale (extremely important, important, neutral, not very important, not important at all). PRO questionnaire 3, collected at the week 1 through month 4 visits, was a dynamic measure of change from baseline, based on subjects' recollection of any perceived change in their feelings of satisfaction with their eyelashes and the study treatment. For the majority of questions, subjects were asked to answer using the provided 5-point scale (eg, very much agree, agree, neutral, disagree, very much disagree). PRO questionnaire 4, collected only during the month 5 visit, asked the subjects to rate their change in overall satisfaction with the appearance of their eyes, with their daily activities, and with their quality of life. Subjects were asked to check 1 box on a 15-point scale ranging from “a very great deal better” to “a very great deal worse.”

Safety measurements collected during this study included adverse events, ophthalmic examination variables (iris color, IOP, visual acuity, biomicroscopy, and ophthalmoscopy), physical examination, vital signs, and pregnancy testing.

Iris color was recorded for each subject at every study visit. Iris color was grouped as light (blue, blue-gray, blue/gray-brown, green, green-brown, hazel, and other) and dark (brown and dark brown). If the “other” category contained black in the description, it was grouped as dark. IOP (measured at approximately the same time of day at each visit), visual acuity, and biomicroscopy data were collected at screening, week 1, and months 1, 2, 3, 4, and 5. Ophthalmoscopy (dilated) was performed at screening and month 4.

The GEA scale with photonumeric guide was developed by Allergan and was determined to be a reliable and reproducible instrument in grading overall eyelash prominence. The digital image analysis performed for the evaluation of eyelash length, thickness/fullness, and darkness was developed by Canfield Scientific, Inc. and was determined to be a reliable and reproducible instrument. The safety measurements evaluated in this study are widely used in clinical studies.

Continuous demographic variables were analyzed using parametric tests (ie, 2-sample t-test). Binary or ordinal demographic variables were analyzed by nonparametric methods (ie, Pearson's chi-square test, Wilcoxon rank-sum test, etc). Ordinal and continuous PRO data were summarized by descriptive statistics and analyzed by the Wilcoxon rank-sum test. Statistical tests were considered statistically significant if 2-sided p-value is 0.05.

Three analysis populations were utilized: the intent-to-treat (ITT) population (primary efficacy analysis population) consisted of all randomized subjects, regardless of whether or not treatment was received or administered; the per-protocol (PP) population (secondary efficacy analysis population) consisted of subjects who had no major deviations from the protocol during their participation in the trial; and the safety population consisted of all subjects who received 1 or more doses of study medication. For the safety and PP populations, statistical analysis was to be based on the actual treatment received. For the ITT population, statistical analysis was to be based on the randomization assignment. The PP population was determined prior to database lock.

The primary efficacy measurement collected during this study was overall eyelash prominence measured using the GEA scale with photonumeric guide (1 [minimal], 2 [moderate], 3 [marked], 4 [very marked], corresponding to frontal and superior eyelash views). For the primary efficacy endpoint, a clinical response was defined as at least a 1-grade increase in the GEA score from baseline at month 4 (week 16). GEA scores were assigned by the investigator based on overall eyelash prominence across both eyes. If data were missing or not available for baseline (day 1), data from the screening visit were used as the baseline value. The proportion of subjects with at least a 1-grade increase from baseline was summarized by a frequency table and analyzed by the Pearson's chi-square test for 2-by-2 tables at each visit. The number and percentage of subjects in each GEA category were summarized by treatment group and visit by a frequency table. No test was performed for treatment-by-center interaction. For the ITT population analysis, if any GEA scores were missing, data imputation was performed by last observation carried forward (LOCF) up to month 4 (week 16). No data imputation was performed for missing values for month 5 (post-treatment). Analysis of efficacy data was applied to both the ITT and PP populations. ITT analyses were based on LOCF; PP analyses were based on observed cases.

The percentage of subjects in each treatment group who experienced at least a 2-grade increase from baseline in GEA score at each study visit was summarized by a frequency table and analyzed by the Pearson's chi-square test for 2-by-2 tables at each visit. Mean change from baseline in GEA score was calculated for each treatment group at each study visit. Within-group comparisons were performed using a Wilcoxon signed-rank test for change from baseline. Between-group comparisons were performed using a Wilcoxon rank-sum test.

For assessments of eyelash length, progressive eyelash thickness/fullness, and eyelash darkness (intensity) based on digital image analysis, analyses were based on the average of the measurements from both left and right upper eyelashes (from the superior view images). For each of these variables, raw values at baseline and change from baseline at each visit were summarized. If baseline (day 1) data are unavailable or if there was a reshoot, then the screening visit digital image analysis data were imputed for the baseline (day 1) data. In the event that a subject's digital image was not able to be interpreted due to the presence of spectral noise, he or she was not included in the analysis population for that particular secondary endpoint. Within-group comparisons were performed using a Wilcoxon signed-rank test for change from baseline. Between-group comparisons were performed using a Wilcoxon rank-sum test. Missing data were imputed up to week 16 using the LOCF method.

To control the type I error rate at 0.05 for multiple secondary efficacy variables, a serial gatekeeping procedure was used with the following order of importance for the secondary variables at month 4 (week 16): 1. upper eyelash length (pixel count, change from baseline) 2. average progressive upper eyelash thickness (percent of detected eyelash thickness to progressive AOI, change from baseline) 3. upper eyelash darkness (darkness [0 to 255 units] within the spline, change from baseline).

To test the robustness of the ITT with LOCF analysis for both the primary and secondary efficacy analyses, 2 sensitivity analyses were performed. First, efficacy analyses were performed on the PP population using observed data. Second, instead of LOCF, missing values were imputed with the median value of the subject's treatment group at each respective visit. In addition, for the primary endpoint of a 1-grade increase in GEA scale, a sensitivity analysis was performed where missing values were treated as treatment failures.

The percentage of subjects with a clinical success (at least a 1-grade increase from baseline in GEA) at month 4 (week 16) were analyzed by subgroups of age (<45, 45 to 65, and >65 years), gender, race (Caucasian and non-Caucasian), baseline GEA score (minimal and moderate), and investigational center. These data were summarized by frequency tables and analyzed by Pearson's chi-square test or Fisher's exact test, as appropriate.

Four PRO questionnaires were collected during this study for the purpose of health outcomes analysis. Analyses of these data were based on the ITT population, with each question analyzed at baseline and follow-up visits (change from baseline).

Within each treatment group, a Wilcoxon signed-rank test for change from baseline was performed. Between-group comparisons were performed using a Wilcoxon rank-sum test. Missing data for questionnaires 1 and 3 were imputed up to week 16 (month 4) using the LOCF method. There was no imputation of data for questionnaires 2 and 4 because these were only collected during 1 study visit (questionnaire 2 at day 1 and questionnaire 4 at month 5).

Safety data (adverse events, ophthalmic examination variables, physical examination, and vital signs variables) were summarized by descriptive statistics and/or frequency tables and were analyzed by appropriate nonparametric statistical methods (Pearson's chi-square test, Wilcoxon rank sum test) and/or parametric tests (ANOVA, t-test). The safety analyses were based on the safety population. No data imputation for missing visits or values was performed.

Iris color, visual acuity, IOP, and biomicroscopy measurements were collected at screening, week 1, and months 1, 2, 3, 4, and 5. Ophthalmoscopy measurements were collected at screening and month 4. Iris color data were summarized by a frequency tabulation and between-group comparisons were performed for iris color as dark versus light using Pearson's chi-square or Fisher's exact test. The number of subjects reporting a shift from light to dark irides from the screening visit to month 4 (or early exit) and from screening visit to month 5 was analyzed; between-group comparisons were performed using Pearson's chi-square or Fisher's exact test.

IOP measurements were collected twice for each eye. If the 2 measurements differed by more than 2 mmHg, a third measurement was taken on that eye. If 2 measurements were collected, the average of the 2 was recorded as the IOP for a particular eye; if a third measurement was collected, the median measurement was recorded as the IOP for that eye. As all subjects were treated bilaterally, the average of the subject's right and left eye IOP values was analyzed for each time point. In the case where data were collected for only 1 eye, the collected data served as the IOP for the subject at that time point. IOP change from baseline was calculated for each scheduled follow-up time point and these data were summarized by descriptive statistics and analyzed by 1-way ANOVA. Within-group changes from baseline were analyzed by the paired t-test. All analyses were based on observed cases and no data imputations were performed.

Best-corrected visual acuity was measured using a logarithmic visual acuity chart and was recorded in Snellen equivalent units on the case report forms. Each subject's final evaluation of visual acuity was compared with baseline. The line number changes in visual acuity was summarized as follows: worse (a decrease of 2 lines or more), no change (a change between −2 and +2 lines), and better (an increase of 2 lines or more). The tabulation was based on the eye with the worst change from baseline (lower value). The exact method for this calculation is described in the statistical analysis plan. The frequency tabulation was analyzed using Pearson's chi-square test or Fisher's exact test where appropriate. Data were summarized by frequency tables for the treatment period and the post-treatment period separately. Biomicroscopy and ophthalmoscopy data were collected using a 5-point scale (0 [none], 0.5 [trace], 1 [mild], 2 [moderate], 3 [severe]) for the following findings: lid/lashes (edema, erythema, hyperemia, other pathology), conjunctiva (edema, erythema, hyperemia, other pathology), cornea (edema, staining/erosion, other pathology), anterior chamber (cells, flare, other pathology), iris/pupil (other pathology), and lens (cataract). These findings, along with “other” findings (pathologies, including vitreous and fundus), coded by preferred term were analyzed as change from baseline for each eye. Frequency tabulations for subjects with a clinically significant change from baseline in at least 1 eye at 1 or more visits were generated by treatment. A clinically significant finding was defined as at least a 1 severity grade increase (worsening) from the screening visit. Between-group comparisons of the frequency distributions were performed using a Pearson's chi-square test or Fisher's exact test, where appropriate.

Lens status data were collected as either phakic, pseudophakic, or aphakic and are presented in a listing.

Physical examinations were performed at the screening visit and at month 4 or early exit. Physical examination findings were summarized by a frequency table for each of these 2 visits.

Vital signs collected during this study were blood pressure and pulse rate, collected at each visit. These data were summarized by descriptive statistics and analyzed by 1-way ANOVA. Analyses at post-baseline visits were based on change from baseline. Within-group changes from baseline were analyzed by the paired t-test. All analyses were based on observed cases and no data imputations were performed. Pregnancy tests were performed and the results are presented in a listing.

All adverse events were summarized and analyzed by the following subgroups: age (<45, 45 to 65, and >65 years), gender, and race (Caucasian and non-Caucasian). This was performed for both the treatment and posttreatment periods. Analyses of IOP were stratified by 3 subgroups, according to baseline IOP (8 to 12 mm Hg, >12 to 15 mm Hg, and >15 mm Hg). These ranges were determined by taking the bottom tercile, middle tercile, and the top tercile of baseline IOPs. The sampling unit was the “eye” (ie, each subject contributed 2 data points). Within each of the subgroups, the number and percent of subjects with an IOP of ≦6 mmHg was summarized by treatment group and visit utilizing a frequency table and was analyzed by the Pearson's chi-square test or Fisher's exact tables for 2-by-2 tables for each visit. The sampling unit was the eye and not the subject. In addition, a scatterplot of baseline IOP versus final IOP for each treatment group was presented for the treatment period and post-treatment period.

Results. A total of 409 subjects were screened for the study, and 278 (68.0%) of these subjects were enrolled. Of the 278 enrolled subjects, 137 subjects were randomized to treatment with bimatoprost 0.03% and 141 to vehicle. A total of 257 subjects (92.4%) completed the study, including 131 subjects (95.6%) in the bimatoprost 0.03% group and 126 subjects (89.4%) in the vehicle group. The most common reason for discontinuation was adverse event. Masking was not broken for any subject at the time of discontinuation.

The ITT population consisted of all 278 randomized subjects, regardless of whether or not treatment was received or administered. This was the primary analysis population used for all efficacy and health outcomes analyses. The PP population consisted of all 278 subjects who had no major deviations from the protocol during their participation in the study. This population was used for selected secondary efficacy analyses. The safety population consisted of all 278 subjects who received at least 1 or more doses of study medication.

The 2 treatment groups were comparable at baseline, with no statistically significant demographic differences. Overall, the mean age of the subjects was 49.8 years (range 22-78 years). The majority of the population was female (97.1%) and Caucasian (80.9%). The majority of subjects had light irides (60.1%). As per inclusion criteria, all enrolled subjects had a baseline GEA score of 1 (20.1%) or 2 (79.9%), with a similar distribution of GEA scores in both treatment groups at baseline. No subjects in either treatment group had baseline GEA scores of 3 (marked) or 4 (very marked).

Use of prestudy medications was reported by 2.2% of subjects, with no notable differences between the 2 treatment groups in the types of medications used or in their frequencies. No prestudy procedures were reported. Overall, the majority of subjects used concomitant medications during both the treatment (77.0%) and post-treatment (73.2%) periods. The categories of medications that were used by more than 10.0% of subjects during the treatment period were multivitamins (14.4%, 40/278), progestogens and estrogens, fixed combinations (14.4%, 40/278), thyroid hormones (11.2%, 31/278), and selective serotonin reuptake inhibitors (10.4%, 29/278), with no notable differences between the 2 treatment groups in the types of medications used or their frequencies. Similar rates of concomitant medication use were reported during the post-treatment period. As per exclusion criteria, no subjects used ocular IOP-lowering drugs or ocular or systemic prostaglandins or prostamides for the duration of the study. Overall, concurrent procedures were reported for 7.6% ( 21/278) and 1.6% ( 4/257) of subjects during the treatment and posttreatment periods. There were no notable differences between the 2 treatment groups in the types of concurrent procedures and in their frequencies. The majority of subjects reported surgical history and 23.7% of subjects reported ophthalmic surgical history. Details of subjects' medical history and ophthalmic medical history were reported. There were no statistically significant differences between the 2 treatment groups (p≧0.102).

The primary efficacy measurement collected during this study was overall eyelash prominence measured using the GEA scale with photonumeric guide (1 [minimal], 2 [moderate], 3 [marked], 4 [very marked], corresponding to frontal and superior eyelash views). GEA scores were assigned based on overall eyelash prominence across both eyes. For the primary efficacy endpoint, a clinical response was defined as at least a 1-grade increase in the GEA score from baseline at month 4. In addition, the percentage of subjects in each treatment group who experienced an improvement in overall eyelash prominence by 2 or more grades on the GEA scale was evaluated. The 3 secondary endpoints were eyelash length, progressive eyelash thickness/fullness, and eyelash darkness, assessed by digital image analysis (superior views) using a serial gatekeeping method of analysis. All primary and secondary efficacy endpoints were met, with subjects in the bimatoprost group experiencing statistically significantly higher rates of improved eyelash prominence (defined by a 1-grade increase on the GEA scale [primary endpoint] and for an additional analysis, defined by the more stringent 2-grade increase on the GEA scale), eyelash length, thickness/fullness, and darkness, as compared to vehicle at week 16 (p<0.0001 for all). The between-group p-values were also statistically significant when the more statistically conservative Bonferroni correction was applied to test each of these 5 pairwise comparisons separately. Only a p-value of less than 0.01 [0.05/5] would provide evidence of a treatment effect; the between-group p-value for each of these 5 endpoints at week 16 was <0.0001.

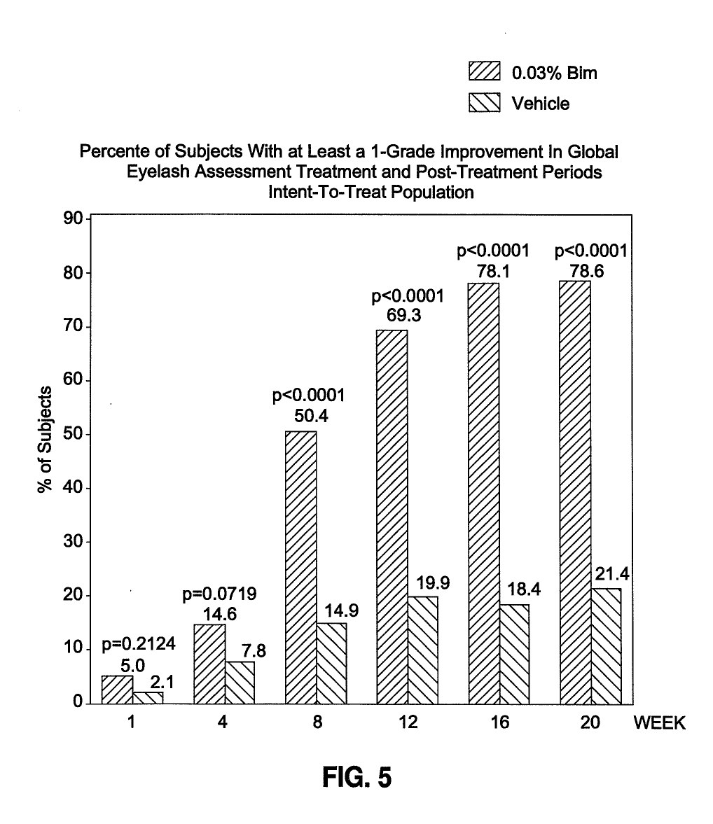

A statistically significantly higher percentage of subjects in the bimatoprost group (78.1%, 107/137) compared with the vehicle group (18.4%, 26/141) experienced at least a 1-grade increase from baseline in overall eyelash prominence as rated by the GEA scale at week 16 (p<0.0001). As early as week 1, a difference in overall eyelash prominence favoring the bimatoprost group was noted. This difference between the 2 treatment groups became more pronounced by week 4, with the bimatoprost group having a higher percentage of subjects with increased eyelash prominence compared with the vehicle group. By week 8, a statistically significant difference in favor of bimatoprost was detected (p<0.0001) and this difference was maintained throughout the duration of the treatment and posttreatment periods. Similar results were observed using the PP population (p<0.0001 by week 8 and beyond). FIG. 5 shows the number (%) of subjects with at least a 1-grade increase from baseline in GEA, treatment and post-treatment periods (ITT Population).

Discussion. Bimatoprost 0.03% solution applied to the upper eyelid margins of healthy adult subjects once per day for 4 months resulted in significant improvements compared to vehicle in the growth of eyelashes, as measured by overall eyelash prominence (defined by at least a 1-grade increase on the GEA scale), eyelash length, thickness/fullness, and darkness. These improvements were statistically significant for the bimatoprost group as compared to the vehicle group for all endpoints by week 8 (statistical significance was first seen for eyelash length at week 4), and were maintained through the duration of the treatment period and 1-month post-treatment period. Bimatoprost was safe and well-tolerated in this population of healthy adult subjects. The adverse event profile was favorable, with only conjunctival hyperemia being reported as an adverse event by a statistically significantly higher percentage of subjects in the bimatoprost group (3.6%, 5/137) compared with the vehicle group (0.0%, 0/141). Three subjects reported serious adverse events during this study, none of which were considered by the investigator to be related to treatment. No subject died during the study.

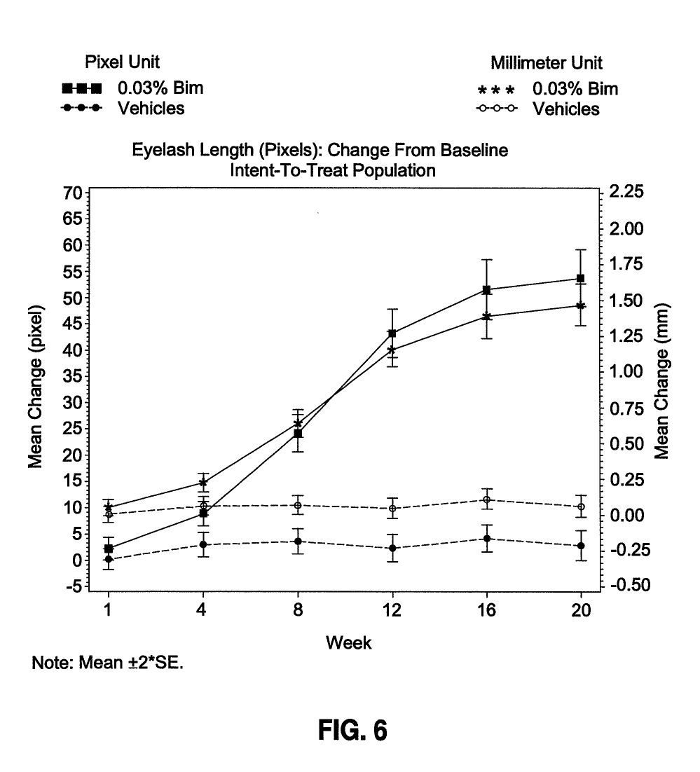

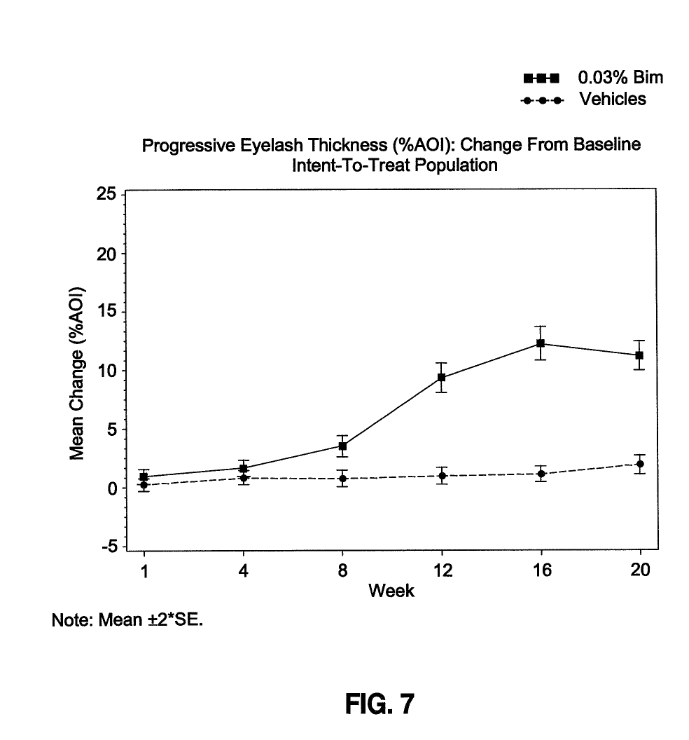

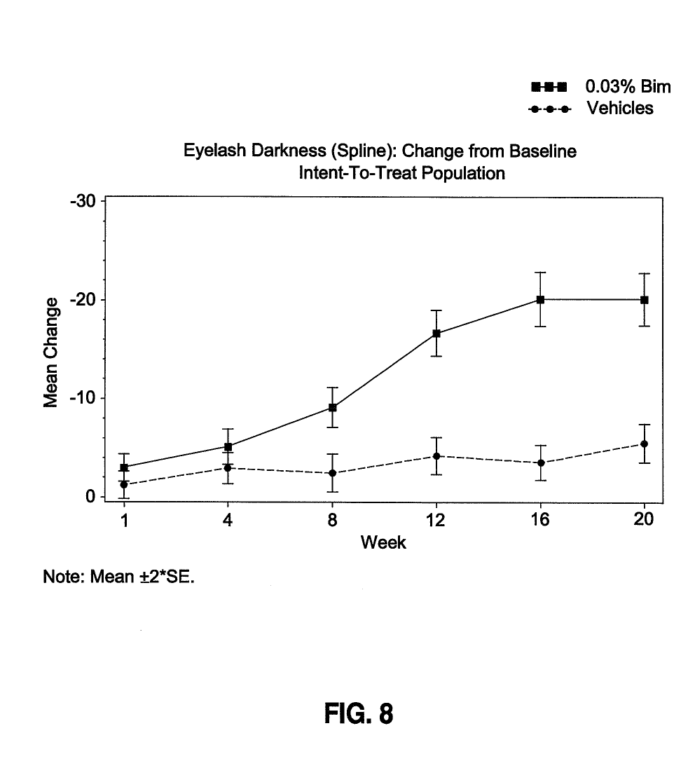

Conclusions. All of the objectives of the study were met, and bimatoprost 0.03% solution was found to be effective, safe, and well-tolerated in this study population of healthy adult subjects. A statistically significantly higher percentage of subjects in the bimatoprost group compared with the vehicle group experienced improved eyelash prominence, defined as at least a 1-grade increase in GEA score, at week 8, week 12, week 16 (primary endpoint), and week 20 (post-treatment period). Furthermore, a statistically significantly higher percentage of subjects in the bimatoprost group compared with the vehicle group experienced the more stringent 2-grade increase in GEA score at week 12, week 16, and week 20. For the 3 secondary efficacy endpoints of eyelash length, progressive eyelash thickness/fullness, and eyelash darkness (assessed using serial gatekeeping), improvements from baseline were statistically significantly more pronounced in the bimatoprost group compared with the vehicle group after 4 weeks of treatment for the measurement of length, and after 8 weeks of treatment for thickness/fullness and darkness. At week 16, subjects had experienced a mean change from baseline in eyelash length corresponding to a 25% and 2% increase in length for the bimatoprost and vehicle groups, respectively (p<0.0001). For eyelash thickness/fullness, at week 16 subjects had experienced a mean change from baseline corresponding to a 106% and 12% increase in thickness/fullness for the bimatoprost and vehicle groups, respectively (p<0.0001) (see FIGS. 6-8). For eyelash darkness, at week 16 subjects had experienced a mean change from baseline corresponding to 18% and 3% darker eyelashes for the bimatoprost and vehicle groups, respectively (p<0.0001). When a Bonferroni correction was applied to test each of the 5 pairwise comparisons separately, differences between the 2 treatment groups were also statistically significant favoring bimatoprost over vehicle at week 16 for improvements in eyelash prominence (defined by both a 1-grade increase and a 2-grade increase on the GEA scale), eyelash length, progressive eyelash thickness/fullness, and eyelash darkness. Only a p-value of less than 0.01 [0.05/5] would provide evidence of a treatment effect; the between-group p-value for each of these 5 endpoints at week 16 was <0.0001. The effects of improved eyelash prominence, length, thickness/fullness, and darkness continued to be evident to a statistically significant degree in the bimatoprost group as compared to vehicle through the 1-month post-treatment follow-up period.

TABLE 3
Interocualr pressure (mmHg)

| | | 0.03% Bimatoprost | Vehicle | |
|---|---|---|---|---|
| | | (N = 274) | (N = 282) | p-values (a) |
| Week | N | 252 | 238 | <0.001 |
| 12 | Mean | −1.54 | −0.64 | |
| | SD | 2.262 | 2.322 | |
| | Median | −1.25 | −0.50 | |
| | Min | −7.0 | −9.5 | |
| | Max | 3.5 | 7.0 | |
| | p-value (b) | <0.001 | <0.001 | |
| Week | N | 252 | 250 | 0.009 |
| 16 | Mean | −1.25 | −0.72 | |
| | SD | 2.195 | 2.317 | |
| | Median | −1.50 | −0.50 | |
| | Min | −7.5 | −10.0 | |
| | Max | 6.0 | 5.0 | |
| | p-value (b) | <0.001 | <0.001 | |
| Week | N | 262 | 252 | 0.118 |
| 20 | Mean | −0.67 | −0.35 | |
| | SD | 2.220 | 2.395 | |
| | Median | −0.50 | −0.50 | |
| | Min | −7.5 | −7.0 | |
| | Max | 5.0 | 6.0 | |
| | p-value (b) | <0.001 | 0.021 | |

Note:
The eye is used as the sampling unit rather than the subject (e.g. average of CD and OS).
(a) A one-way analysis of variance was performed to evaluate the difference among/between treatment groups.
(b) Paired t-tests were used to test for mean shifts from baseline within treatment groups

Results of the studies described in Example 2 illustrate that the compositions described herein can be safe and effective in treatment of hair in at least one eyebrow region.

### Example 3

### Bimatoprost to Aid in Eyebrow Growth

A 61 year old woman participates in a clinical study for eyebrow growth. The patient has lost both her eyebrows as a result of recent treatments of chemotherapy.

Bimatoprost 0.03% is applied along the eyebrow line of both eyes. After approximately 4 weeks, the woman begins to experience eyebrow growth. Following the study, the patient woman has full, long eyebrows. The woman is extremely satisfied with the results and no longer needs to apply make-up in place of real eyebrows.

### Example 4

### Topical Cream

A topical cream containing 1.0% bimatoprost is prepared as follows: Tegacid and spermaceti are melted together at a temperature of 70-80° C. Methylparaben is dissolved in about 500 g of water and propylene glycol, polysorbate 80, and bimatoprost are added in turn, maintaining a temperature of 75-80° C. The methylparaben mixture is added slowly to the Tegacid and spermaceti melt, with constant stirring. The addition is continued for at least 30 minutes with additional stirring until the temperature has dropped to 40-45° C. Finally, sufficient water is added to bring the final weight to 100 g and the preparation stirred to maintain homogeneity until cooled and congealed.

The composition is applied to an eyebrow or scalp at least once daily to stimulate the growth of hair.

### Example 5

### Topical Ointment

An ointment containing 2% by weight bimatoprost is prepared as follows: White petrolatum and wool fat are melted, strained and liquid petrolatum is added thereto. The bimatoprost, zinc oxide, and calamine are added to the remaining liquid petrolatum and the mixture milled until the powders are finely divided and uniformly dispersed. The mixture is stirred into the white petrolatum, melted and cooled with stirring until the ointment congeals.

The foregoing ointment can be applied topically to an eyebrow for increased rate of hair growth, and can be prepared by omitting the zinc oxide and calamine.

### Example 6

### Ointment

A dermatological ophthalmic ointment containing 10% by weight bimatoprost is prepared by adding the active compound to light liquid petrolatum. White petrolatum is melted together with wool fat, strained, and the temperature adjusted to 45-50° C. The liquid petrolatum slurry is added and the ointment stirred until congealed. Suitably the ointment is packaged in 30 g tubes.

The foregoing ointment can be applied to the eyebrows to enhance the growth of hair.

### Example 7

### Solution

An aqueous solution containing 5%, by weight, bimatoprost is prepared as follows. Bimatoprost is dissolved in water and the resulting solution is sterilized by filtration. The solution is aseptically filled into sterile containers.

The composition so prepared can be used in the topical treatment of eyebrows by application to at least one eyebrow region once daily.

### Example 8

### Lotion

A sample of bimatoprost is dissolved in the vehicle of N-methylpyrrolidone and propylene glycol. The composition can be used for application to the eyebrows to aid in hair growth.

### Example 9

### Aerosol

An aerosol containing approximately 0.1% by weight bimatoprost is prepared by dissolving the bimatoprost in absolute alcohol. The resulting solution filtered to remove particles and lint. This solution is chilled to about minus 30° C. To the solution is added a chilled mixture of dichlorodifluoromethane and dichlorotetrafluoroethane.

Thirteen mL plastic-coated amber bottles are cold filled with 11.5 g each of the resulting solution and capped.

The composition can be sprayed on the eyebrows daily to stimulate the growth of hair.

### Example 10

### Dusting Powder

A powder of the compound bimatoprost is prepared by mixing in dry form with talcum powder at a weight/weight ratio of 1:10. The powdered mixture is dusted on the eyebrows for increased rate of hair growth.

### Example 11

### Related Compounds

Following the procedure of the preceding Examples, compositions are similarly prepared substituting an equimolar amount of a compound of Table 1 for the bimatoprost disclosed in the preceding Examples. Similar results are obtained.

### Example 12

### Bimatoprost to Aid in Scalp Hair Growth

A 61 year old woman participates in a clinical study for scalp growth. The patient has lost significant amounts of hair on her head as a result of recent treatments of chemotherapy. Bimatoprost 1.0% cream according to the teachings of Example 4 is applied twice a day to the 61 year old patient's scalp in areas which have experienced hair loss. After approximately 3-4 weeks, the woman begins to experience hair growth. After 8 weeks, a clinically significant amount of hair growth occurs in areas on the scalp where the patient lost her hair due to chemotherapy treatments. The patient continues to apply the 1.0% cream until all of her hair lost due to chemotherapy returns.

While the preferred embodiment of the invention has been illustrated and described, it will be appreciated that various changes can be made therein without departing from the spirit and scope of the invention.

What is claimed:

1. A method for treating alopecia in an individual in need thereof comprising the step of administering a therapeutically effective amount of a composition comprising a prostamide, prodrug thereof, salt thereof, or mixtures thereof to the individual, wherein the administration results in a reduction in alopecia.

2. The method of claim 1, wherein the prostamide is bimatoprost, a prostamide D₂, prostamide E₂, a prostamide F_{2α}, a 11β-prostamide F_{2α}, a prostamide G₂, a prostamide H₂, or a prostamide I₂.

3. The method of claim 1, wherein the alopecia is scarring alopecia or non-scarring alopecia.

4. The method of claim 3, wherein the scarring alopecia is associated with a disorder selected from the group consisting of hair loss due to chemotherapy, a bullous disease, a chemical exposure, a discoid lupus erythematosus, a severre folliculitis, a lichen planopilaris, a dissecting cellulitis, a central centrifugal cicatricial alopecia, a postmenopausal frontal fibrosing alopecia, a tumor, and a skin outgrowth.

5. The method of claim 3, wherein the non-scarring alopecia is selected from the group consisting of anagen effluvium, alopecia adnata, alopecia androgenetica, alopecia areata, alopecia congenitalis, alopecia diffusa, alopecia disseminate, alopecia follicularis, alopecia leprotica, alopecia marginalis, alopecia medicamentosa, alopecia mucinosa, alopecia neurotica, alopecia pityrodes, alopecia presenili, alopecia senilis, alopecia symptomatica, alopecia syphilitica, alopecia totalis, alopecia toxica, alopecia triangularis, alopecia triangularis congenitalis, alopecia universalis, folliculitis, olliculitis decalvans, traction alopecia, trichotillomania, telogen effluvium, and inherited disorder of the hair shaft.

6. The method for claim 4 wherein the hair loss is due to chemotherapy and the hair loss is associated with eyelashes, eyebrows and scalp hair.

7. The method of claim 6, wherein the prostamide is bimatoprost.

8. The method of claim 7 wherein the method restores natural hair color.

11. The method of claim 7 wherein the bimatoprost is in solution form and is applied 0.2 cc bimatoprost applied twice daily.

12. The method of claim 7 wherein the solution is 0.03% w/v bimatorprost.

13. The method of claim 7 wherein the bimatoprost is in cream form.
